SEQUENCE LISTING (16 sequences)

SEQ ID NO: 1 - DNA, 20 bp, synthetic construct
  source 1..20: mol_type = other DNA; note = SEQ ID N 1 - NKX3.1 Forward
tccgtcttttggctctgagt

SEQ ID NO: 2 - DNA, 20 bp, synthetic construct
  source 1..20: mol_type = other DNA; note = SEQ ID N 2 - NKX3.1 Reverse
gtgaaagtgcacgctgaaaa

SEQ ID NO: 3 - DNA, 19 bp, synthetic construct
  source 1..19: collected_by = SEQ ID N 3 - R18S Forward; mol_type = other DNA
accgcagctaggaataatg

SEQ ID NO: 4 - DNA, 19 bp, synthetic construct
  source 1..19: mol_type = other DNA; note = SEQ ID N 4 - R18S Reverse
gcctcagttccgaaaacca

SEQ ID NO: 5 - DNA, 20 bp, synthetic construct
  source 1..20: mol_type = other DNA; note = SEQ ID N 5 - ALDH1A3 Forward
gggtcacactggagctagga

SEQ ID NO: 6 - DNA, 19 bp, synthetic construct
  source 1..19: mol_type = other DNA; note = SEQ ID N 6 - ALDH1A3 Reverse
ctggcctcttcttggcgaa

SEQ ID NO: 7 - DNA, 22 bp, synthetic construct
  source 1..22: mol_type = other DNA; note = SEQ ID N 7 - PPAPA2A Forward
agaggggctttttctgtactga

SEQ ID NO: 8 - DNA, 21 bp, synthetic construct
  source 1..21: mol_type = other DNA; note = SEQ ID N 8 - PPAPA2A Reverse
tatacgggacgggatggtact

SEQ ID NO: 9 - DNA, 20 bp, synthetic construct
  source 1..20: mol_type = other DNA; note = SEQ ID N 9 - R.gnavus Forward
tgggtgtaaagggagcgtag

SEQ ID NO: 10 - DNA, 20 bp, synthetic construct
  source 1..20: mol_type = other DNA; note = SEQ ID N 10 - R.gnavus Reverse
ctctccgacactctagcctg

SEQ ID NO: 11 - DNA, 20 bp, synthetic construct
  source 1..20: mol_type = other DNA; note = SEQ ID N 11 - B.acidifaciens Forward
ttcggtatgggatggggatg

SEQ ID NO: 12 - DNA, 20 bp, synthetic construct
  source 1..20: mol_type = other DNA; note = SEQ ID N 12 - B.acidifaciens Reverse
catccttcacgctacttggc

SEQ ID NO: 13 - DNA, 17 bp, synthetic construct
  source 1..17: mol_type = other DNA; note = SEQ ID N 13 - EUB16S Forward
ggtgaatacgttcccgg

SEQ ID NO: 14 - DNA, 19 bp, synthetic construct
  source 1..19: mol_type = other DNA; note = SEQ ID N 14 - EUB16S Reverse
tacggctaccttgttacga

SEQ ID NO: 15 - DNA, 20 bp, synthetic construct
  source 1..20: mol_type = other DNA; note = SEQ ID N 15 8F primer
agagtttgatcctggctcag

SEQ ID NO: 16 - DNA, 17 bp, synthetic construct
  source 1..17: mol_type = other DNA; note = SEQ ID N 16 - 534R primer
attaccgcggctgctgg

(19) United States
(12) Patent Application Publication
(10) Pub. No.: US 2025/0268958 A1
(43) Pub. Date: Aug. 28, 2025

(54) COMMENSAL BACTERIA PROMOTE ENDOCRINE-RESISTANCE IN PROSTATE CANCER VIA ANDROGEN BIOSYNTHESIS

(71) Applicant: FONDAZIONE PER L'ISTITUTO ONCOLOGICO DI RICERCA (IOR), Bellinzona (CH)

(72) Inventors: Andrea ALIMONTI, Bellinzona (CH); Nicolò PERNIGONI, Bellinzona (CH); Elena ZAGATO, Milano (IT); Arianna CALCINOTTO, Bellinzona (CH); Martina TROIANI, Bellinzona (CH)

(73) Assignee: FONDAZIONE PER L'ISTITUTO ONCOLOGICO DI RICERCA (IOR), Bellinzona (CH)

(21) Appl. No.: 18/684,312

(22) PCT Filed: Aug. 1, 2022
(86) PCT No.: PCT/IB2022/057118

§ 371 Date: Feb. 16, 2024

(30) Foreign Application Priority Data
Aug. 17, 2021 (IT) 102021000021974

(51) Int. Cl. A61K 35/747; A61K 31/43; A61K 31/7042; A61K 35/742; A61K 45/06; A61P 35/00

(52) U.S. Cl. CPC A61K 35/747; A61K 31/43; A61K 31/7042; A61K 35/742; A61K 45/06; A61P 35/00

(57) ABSTRACT

The present invention relates to an in vitro and/or ex vivo method for the prognosis of prostate cancer in a subject and/or for determining if a subject suffering from prostate cancer is responsive to a therapeutic treatment, as well as to prebiotic mixtures for preventing, treating and/or inhibiting the development of prostate cancer.

The present invention relates to an in vitro and/or ex vivo method for the prognosis of prostate cancer in a subject and/or for determining if a subject suffering from prostate cancer will be responsive to a therapeutic treatment, as well as to probiotic mixtures for preventing, treating and/or inhibiting the development of prostate cancer.

### STATEMENT REGARDING SEQUENCE LISTING

The instant application contains a Sequence Listing which has been submitted electronically in XML format and is hereby incorporated by reference in its entirety. Said XML copy, created on Sep. 4, 2024 is named 4679_0020001_Seqlisting_ST26.xml and is 18,339 bytes in size.

### STATE OF THE ART

Prostate cancer is the commonest cancer in male. Castration resistance prostate cancer (CRPC, the most aggressive phenotype) invariably emerges despite treatments during disease progression. The emergence of CRPC is a major therapeutic challenge.

Androgen deprivation therapy (ADT) remains the mainstay of treatment for patients suffering from advanced prostate cancer. However, after an initial response, patients develop resistance to ADT and progress. Treatments for CRPC remain unsatisfactory, and this remains invariably lethal. While there is an urgent need for effective treatments for CRPC patients, treatments that delay the onset of CRPC in hormone-sensitive prostate cancer (HSPC) patients treated with ADT may also offer an alternative therapeutic strategy. Microbiota comprises the microorganisms that live in close contact with the host, usually with mutual benefit one another. This relationship is described as symbiotic and is fundamental for the fitness of the host. Perturbations of this equilibrium can occur under pathological conditions, including cancer. Microbiota can directly impact tumor initiation through releasing of toxins or influencing tumor cells through bacterial metabolites. In addition to this, the microbiota can contribute to tumor development through the promotion of inflammation and shaping the tumor immune response. Increasing evidence shows that the microbiota is important for the antitumor activity of both chemotherapy and immune check-point inhibitors, and microbiota modulation might enhance treatment response. Previous findings in mouse models and human prostate tumor samples report the existence of a prostatic microbiota that can support prostate tumor growth by promoting chronic inflammation. In contrast, only a limited number of correlative studies have investigated the role of the gut microbiota in prostate cancer initiation and progression. Given the role played by the gut microbiota in cancer, an intriguing hypothesis is that the intestinal microbiota of patients suffering from prostate cancer could also participate in the host's hormone metabolism (H. Neuman, J. W. Debelius, R. Knight, O. Koren, Microbial endocrinology: the interplay between the microbiota and the endocrine system. FEMS Microbiol Rev 39, 509-521 (2015)), thus impacting prostate cancer growth.

However, up to now the contribution of the gut microbiota to the emergence of CRPC has not been addressed yet.

In this context, there is therefore an urgent need for predictive biomarkers that could enable tailoring treatments for prostate cancer to individual patients, as wells as for effective therapeutic solutions that could delay or prevent the emergence of CRPC, contrasting endocrine-resistance.

### SUMMARY OF THE INVENTION

The present invention is based on the discovery that androgen deprivation in mice and humans drives the expansion of a peculiar intestinal microbiota, and that the gut microbiota contributes to the onset of castration-resistance in subjects suffering from prostate cancer by contributing to the host's androgen metabolism.

Specifically, the authors of the present invention have found that intestinal microbial community in mice and patients with CRPC was enriched for species capable of converting androgen precursors into active androgens. The results summarized in the experimental section of the present specification revealed that the commensal gut microbiota contributes to endocrine resistance in CRPC by providing an alternative source of androgens.

The milestone at the basis of the present invention is hence the demonstration that these particular species can contribute to androgen metabolism, prostate cancer growth, endocrine treatment resistance, and disease outcome.

Notably, the inventors have identified a fecal bacterial signature that associates with prostate cancer patients' overall survival. This signature could be used as a minimally invasive biomarker to identify patients that could benefit from microbiota manipulation strategies. In particular, the inventors have identified gut-associated bacteria Ruminococcus sp. DSM_100440, Ruminococcus sp. OM05_10BH, Streptococcus vestibularis, Clostridiales bacterium VE202_14, Sellimonas intestinalis and Drancourtella massiliensis as unfavorable species. These species can be prognostic of unfavorable clinical outcome and response to standard of care (SOC) treatments. On the other hand, the inventors have identified Prevotella sp. BCRC_81118, Prevotella sp. Marseille_P4119, Prevotella sp. 885 and Prevotella stercorea as associated with a more favorable outcome. Moreover, by testing Prevotella stercorea, Lactobacillus (L. casei, L. buchneri, L. acidophilus, L. paracasei, L. bulgaricus, L. rhammosum) and Bifidobacterium (B. bifidum, B. longum, B. breve) strains alone or in combination in a preclinical model, the inventors demonstrated that probiotic and/or bacterial consortium administration were able to limit expansion of unfavorable microbiota and control tumor growth. These bacteria may be hence used as adjuvant therapy and/or as indicators of a better outcome and response to treatments.

Hence, a first object of the present invention refers to an in vitro and/or ex vivo method for the diagnosis and/or prognosis of prostate cancer in a subject and/or for determining if a subject suffering from prostate cancer is responsive to a therapeutic treatment, comprising:
a. determining the presence of one or more bacteria selected from the group consisting of the genus Ruminococcus, the genus Streptococcus, the genus Clostridiales, the genus Prevotella, the genus Sellimonas and the genus Drancourtella in a biological sample isolated from said subject.

A further object of the invention is an in vitro and/or ex vivo method for monitoring the response of a subject suffering from prostate cancer to a therapeutic treatment, said method comprising the steps of:
a. determining and/or quantifying the levels of one or more bacteria selected from the group consisting of the genus Ruminococcus, the genus Streptococcus, the genus Clostridiales, the genus Prevotella, the genus Sellimonas and the genus Drancourtella in a biological sample isolated from the subject before said treatment;
a′. determining and/or quantifying said levels in a biological sample from the subject after said treatment; and
b. based on the results of the determinations and/or quantifications performed in steps a. and a′. determining if the subject responds to said treatment.

Other objects of the present invention are a kit comprising reagents for determining the presence of one or more bacteria selected from the group consisting of the genus Ruminococcus, the genus Streptococcus, the genus Clostridiales, the genus Prevotella, the genus Sellimonas and the genus Drancourtella in a biological sample isolated from a subject suffering from prostate cancer, preferably CRPC, and the use of said kit for the in vitro and/or ex vivo diagnosis and/or prognosis of prostate cancer in a subject, preferably of castration-resistant prostate cancer, and/or for determining if a subject suffering from prostate cancer is responsive to a therapeutic treatment, according to the method disclosed herein.

Objects of the present invention are also a probiotic mixture comprising at least one bacterial strain selected from the group consisting of the genus Ruminococcus, the genus Streptococcus, the genus Clostridiales, the genus Sellimonas, the genus Drancourtella and a probiotic mixture comprising at least one bacterial strain selected from the group consisting of the genus Prevotella, the genus Lactobacillus and the genus Bifidobacterium, for use for preventing, treating and/or inhibiting the development of prostate cancer, preferably CRPC, and/or androgen deficiencies.

Further objects of the invention are a composition comprising the probiotic mixture as herein disclosed in association with one or more of the following antibiotics: vancomycin, ampicillin, neomycin, and/or metronidazole and a pharmaceutical composition comprising vancomycin, ampicillin, neomycin, and/or metronidazole for use for preventing, treating and/or inhibiting the development of prostate cancer, preferably castration-resistant prostate cancer.

### DETAILED DESCRIPTION OF THE FIGURES

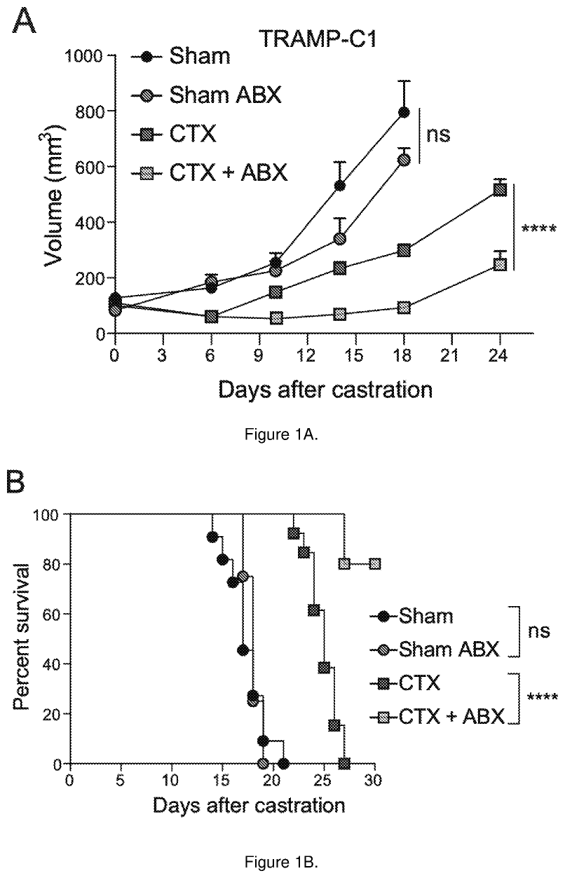

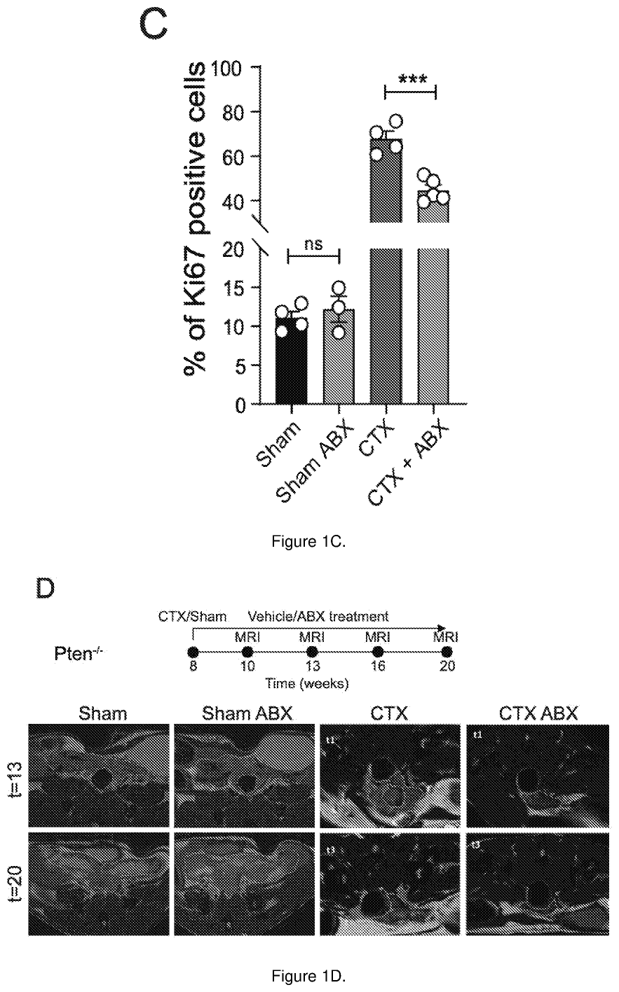

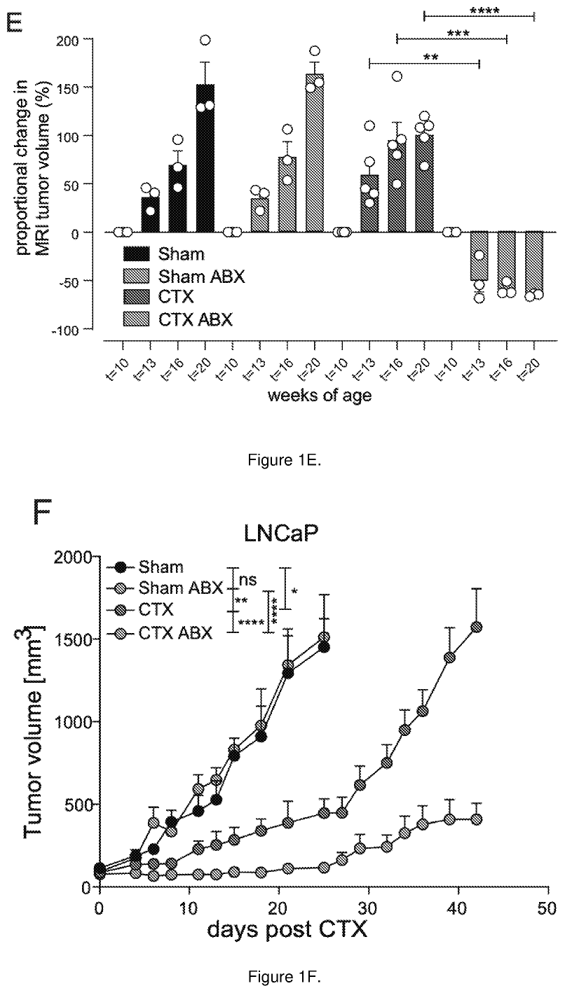

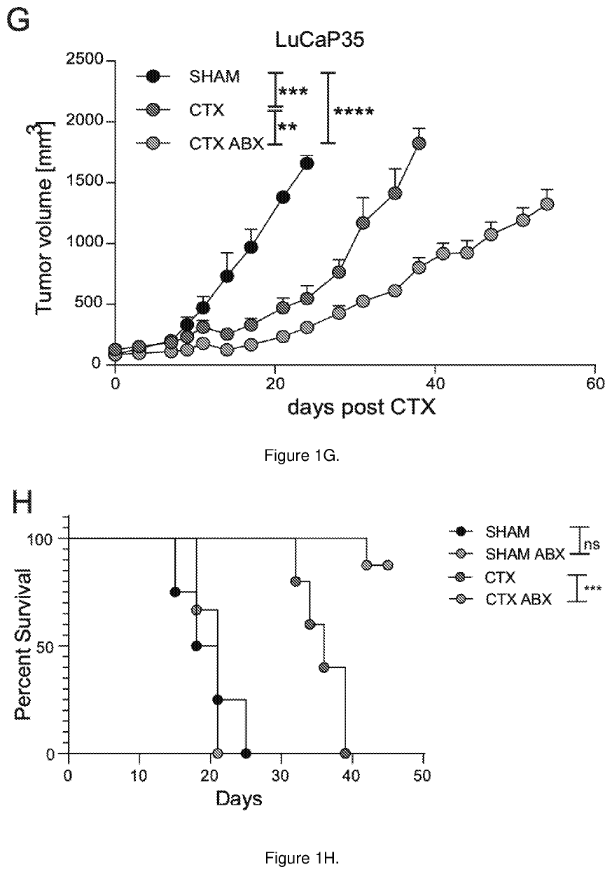

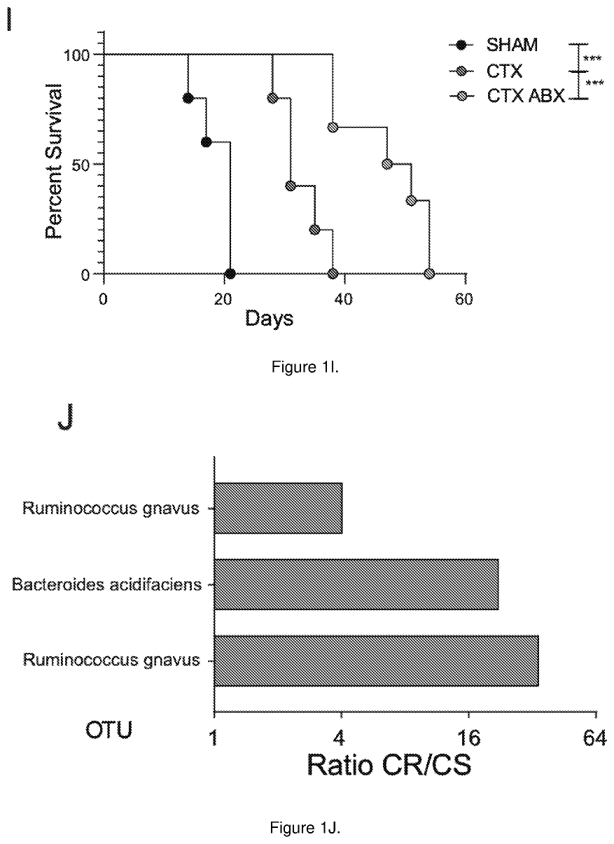

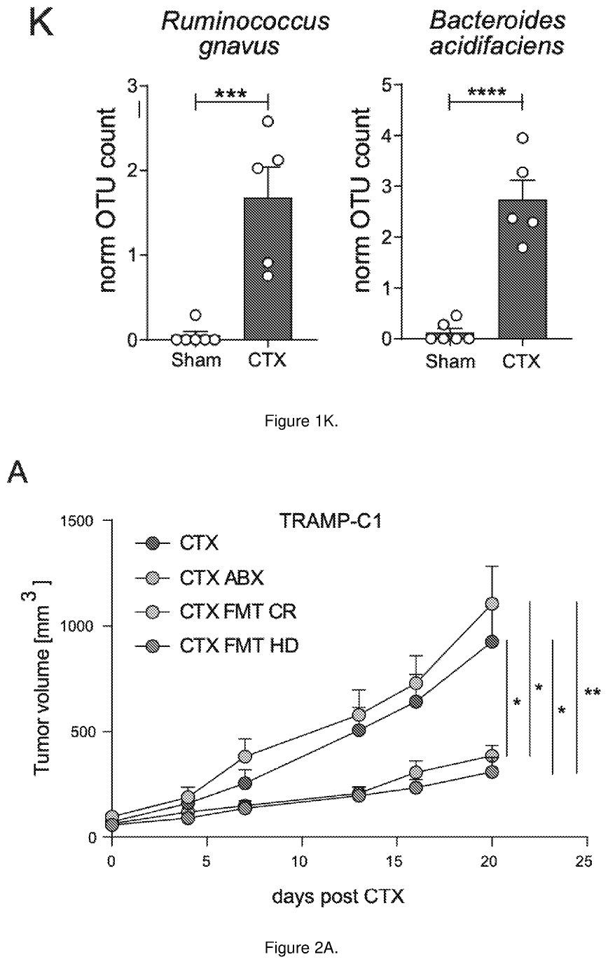

FIG. 1.—Depletion of the intestinal microbiota in castrated but not in sham-operated mice affects CRPC growth. C57BL6/N mice were challenged s.c. with 2.5×10⁶ TRAMP-C1 cells. When tumors became palpable, mice were either castrated (CTX) or sham-operated (sham). After surgery, mice were either administered normal drinking water or antibiotic cocktail (ABX: neomycin 1 g/L, ampicillin 1 g/L, vancomycin 0.5 g/L and metronidazole 2 mg/mouse every other day). (A) Tumor volume (Sham n=14, Sham ABX n=4, CTX n=20, CTX ABX n=10) and (B) survival curve. (C) Percentage of Ki67 positive cells (Sham n=4, Sham ABX n=3, CTX n=4, CTX ABX n=5). Pten^{pc-/-} mice were sham-operated or castrated at 9 weeks of age and either left untreated or treated with ABX; prostatic tumor volume was monitored through serial magnetic resonance imaging (MRI) over a time-course. (D) Experimental scheme and representative MRIs of sham, sham ABX, CTX or CTX ABX mouse at 13 and 20 weeks of age and (E) waterfall plot representing proportional change in tumor size for sham (n=3), sham ABX (n=3), CTX (n=5) and CTX ABX (n=3) Pten^{pc-/-} mice. NRG mice were challenged with LNCaP or LuCaP35, castrated or sham-operated when the tumor was palpable and either left untreated or treated with ABX. (F) Tumor volume and (H) survival curve of LNCaP allograft (Sham n=4, Sham ABX n=3, CTX n=5, CTX ABX n=7). (G) Tumor volume and (1) survival curve of LuCaP35 allograft (Sham n=5, CTX n=6, CTX ABX n=6). The composition of the CS and CR microbiota was analyzed through 16S rDNA sequencing of the feces of Sham (n=6) and CR (n=5) Pten^{pc-/-} mice. (J) Waterfall plot representing the abundance of the significantly different OTUs identified at species level as a ratio CR/CS. (K) Normalized OTU counts relative to Ruminococcus gnavus and Bacteroides acidifaciens (Sham, n=6; CR, n=5). Statistical analysis was performed with: (A), (F), (G) two-way ANOVA and Sidak's multiple comparison test; (B), (H), (1) Mantel Cox test; (C), (E), (K) unpaired two-sided Student's t-test. NS, not significant; *P<0.05; **P<0.01; ***P<0.001; ****P<0.0001.

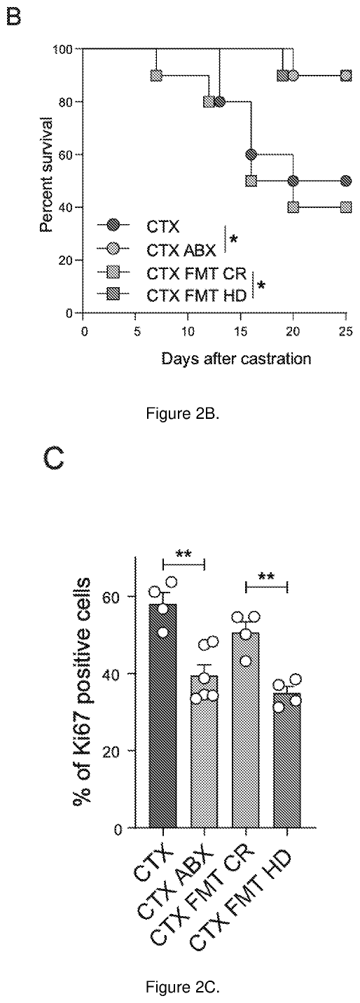

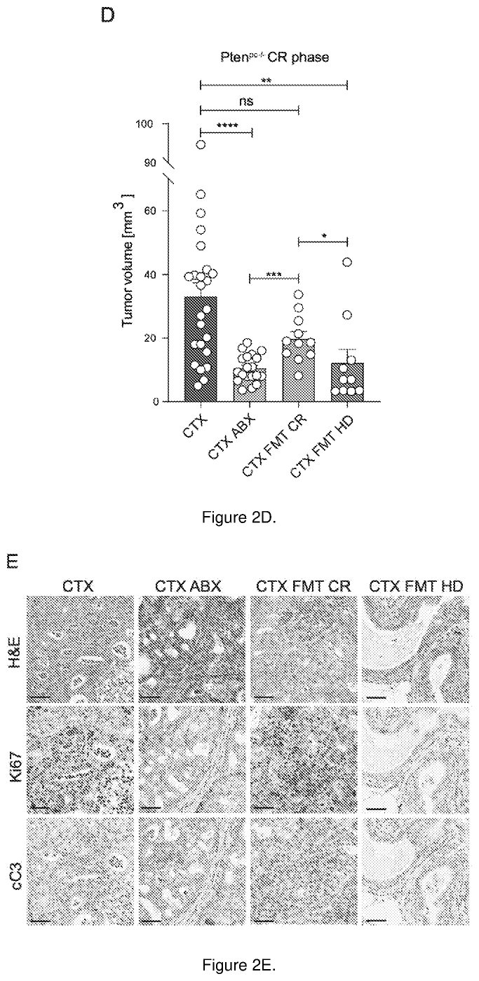

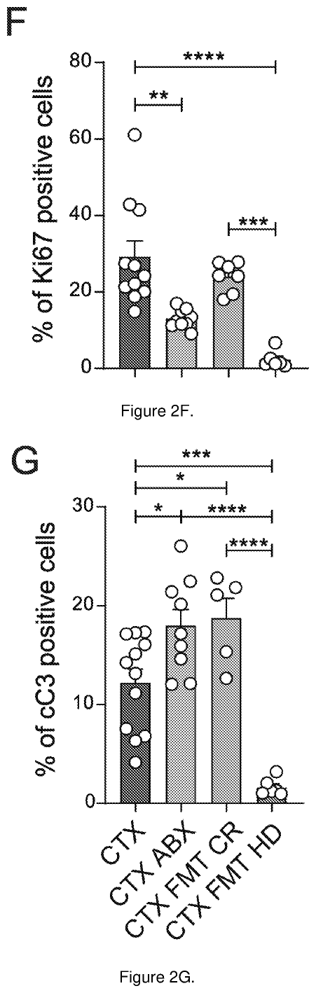

FIG. 2—Fecal microbiota transplantation from CRPC mice supports tumor growth in castrated recipient mice. TRAMP-C1 allograft mice were treated with ABX for 7 days before CTX, then either left untreated (n=9), treated with ABX (n=9) or received FMT from HD (n=8) or CR (n=9) mouse donors. (A) Tumor volume, (B) survival curve and (C) IHC quantification of Ki67 staining (CTX n=4, CTX ABX n=6, CTX FMT CR n=4, CTX FMT HD n=4). Pten^{pc-/-} mice were treated with ABX for 7 days before CTX, then either left untreated, treated with ABX or received FMT from CR or HD donors. (D) AP tumor volume at 20 weeks of age (CTX n=24, CTX ABX n=19, CTX FMT CR n=11, CTX FMT HD n=10). (E) Representative H&E, Ki67 and cC3 IHC staining in AP lobes of Pten^{pc-/-} mice at 20 weeks of age; Scale bar 45 μm. Quantification of (F) Ki67 (CTX n=11, CTX ABX n=9, CTX FMT CR n=7, CTX FMT HD n=6) and (G) cC3 positive cells (CTX n=12, CTX ABX n=9, CTX FMT CR n=5, CTX FMT HD n=6). (H) Histopathological score of AP lobes of CTX (n=6), CTX ABX (n=5), CTX FMT CR (n=5) and CTX FMT HD (n=5) Pten^{pc-/-} mice at 20 weeks of age. (I) Tumor volume of TRAMP-C1 allograft mice treated with ABX for 7 days before CTX, then either left untreated (n=8) or treated with R. gnavus (n=5) via oral gavage. CTX FMT HD was used as internal control. Statistical analysis was performed with: (A) two-way ANOVA and Sidak's multiple comparison test; (B) Mantel Cox test; (C), (H), (1) unpaired two-sided Student's t-test; (D) Mann-Whitney test; (F), (G) one-way ANOVA and Tukey's multiple comparison test; NS, not significant; *P<0.05; **P<0.01; ***P<0.001; ****P<0.0001.

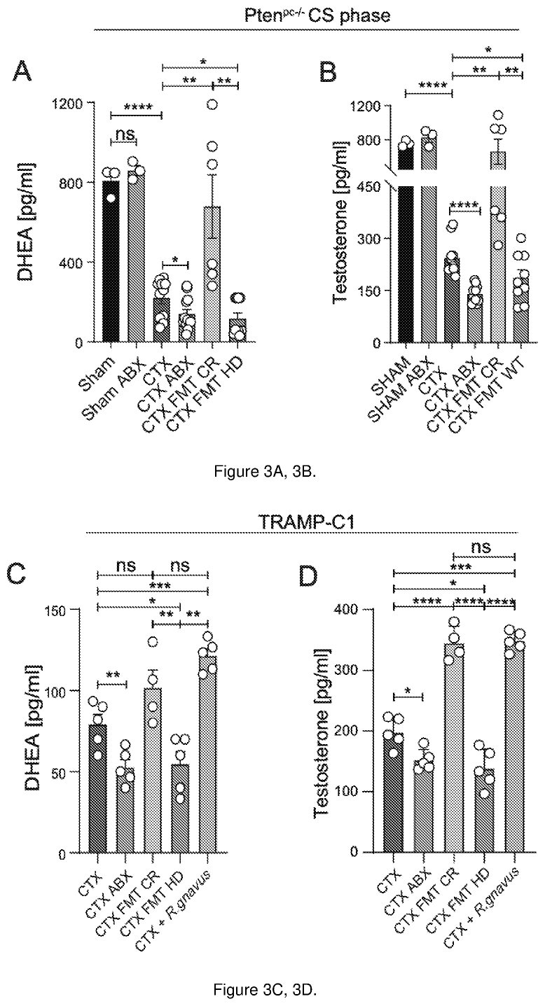

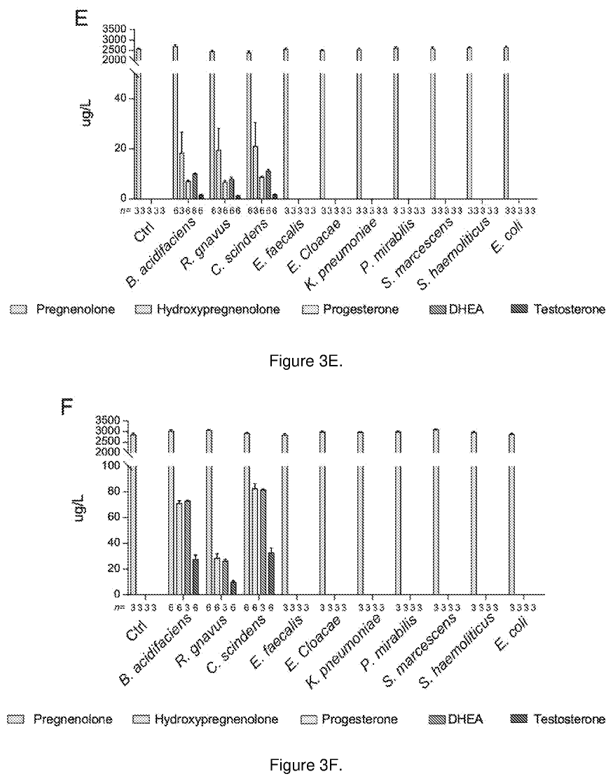

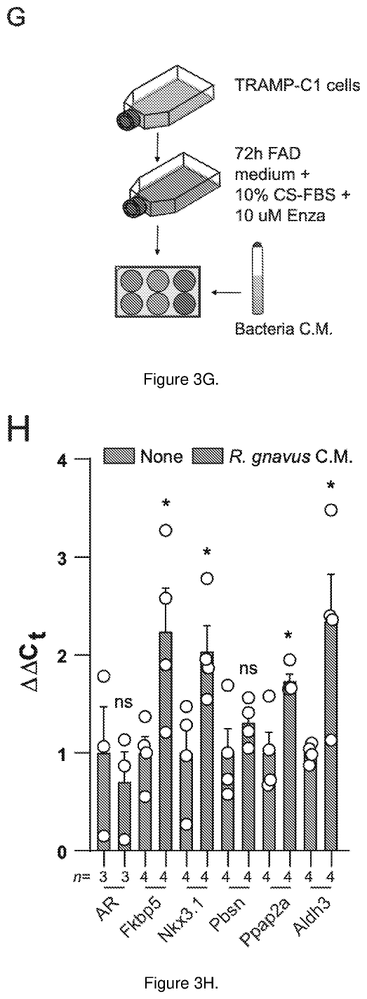

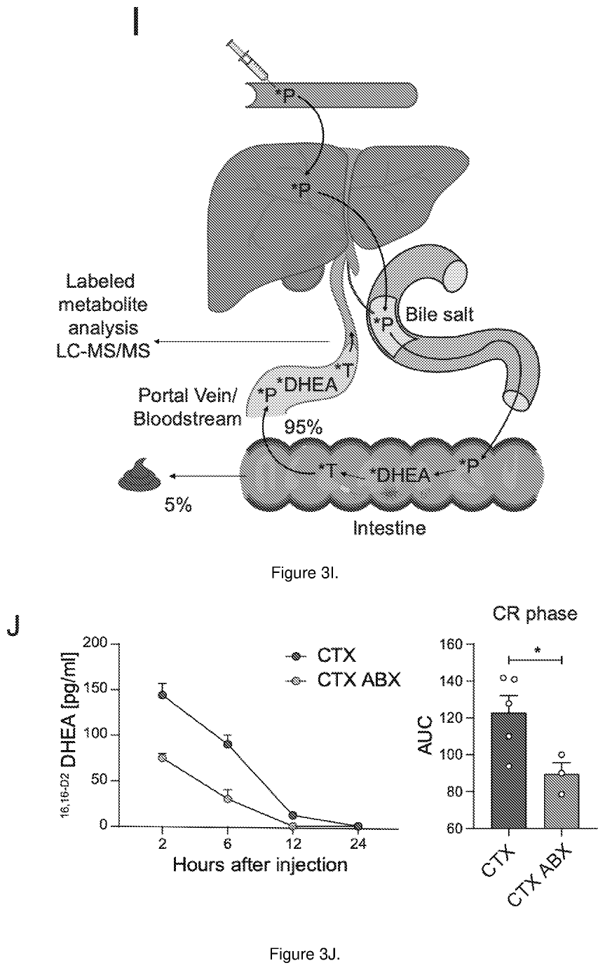

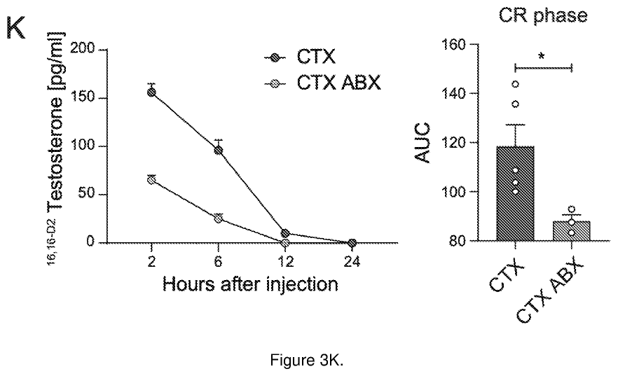

FIG. 3—The CRPC microbiota is enriched in bacterial species that produce androgens and impact on tumor cell growth. Targeted metabolomic analysis was performed in sera of Pten^{pc-/-} mice CTX, CTX ABX, CTX FMT CR and CTX FMT HD at 13 weeks of age. Levels of (A) DHEA and (B) testosterone (Sham n=3, Sham ABX n=3 CTX n=11, CTX ABX n=12, CTX FMT CR n=6, CTX FMT HD n=10). Targeted metabolomic analysis was performed in sera of TRAMP-C1 mice CTX, CTX ABX, CTX FMT CR and CTX FMT HD 18 days after castration. Levels of (C) DHEA and (D) testosterone (CTX n=5, CTX ABX n=5, CTX FMT CR n=4, CTX FMT HD n=5, CTX+R. gnavus n=5). B. acidifaciens, R. gnavus, C. scindens, E. faecalis, E. cloacae, K. pneumoniae, P. mirabilis, S. marcescens, S. haemoliticus, E. coli, were cultured for 48 h in the presence of the indicated androgen precursors in anaerobic conditions and analyzed for production of androgens or androgen precursors through LC-MS/MS. Quantification of androgen pathway intermediates in bacterial conditioned media (CM) after (E) pregnenolone and (F) hydroxypregnenolone treatment; ctrl means no bacteria. TRAMP-C1 cells in full androgen deprivation (FAD) were treated with C. M. of R. gnavus pre-incubated with pregnenolone or TSB for 48 h and analyzed with qRT-PCR. (G) Experimental scheme (CS-FBS: charcoal-stripped FBS; Enza: Enzalutamide) and (H) qRT-PCR analyses of the indicated genes. Pten^{pc-/-} mice treated or not with ABX were injected i.v. with 75 ng of ^{D}pregnenolone. Serum was sampled at 2, 6, 12 and 24 h after the injection and analyzed for the presence of deuterated androgen molecules. (1) Experimental scheme. Time-course quantification and AUC of (J) ^{D}DHEA (CTX n=5, CTX ABX n=3) and (K) ^{D}testosterone (CTX n=5, CTX ABX n=3). Statistical analysis was performed with: (A), (B), (C), (D) one-way ANOVA and unpaired two-sided Student's t-test; (H), (J), (K), unpaired two-sided Student's t-test. NS, not significant; *P<0.05; **P<0.01; ***P<0.001; ****P<0.0001.

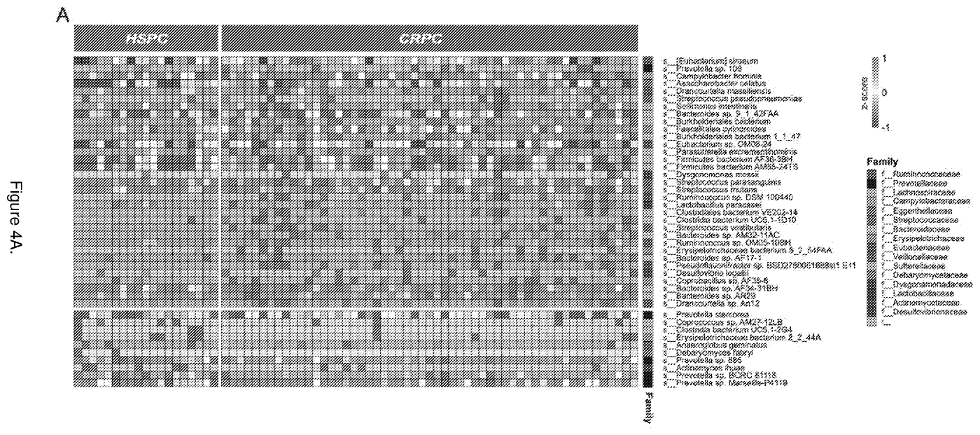

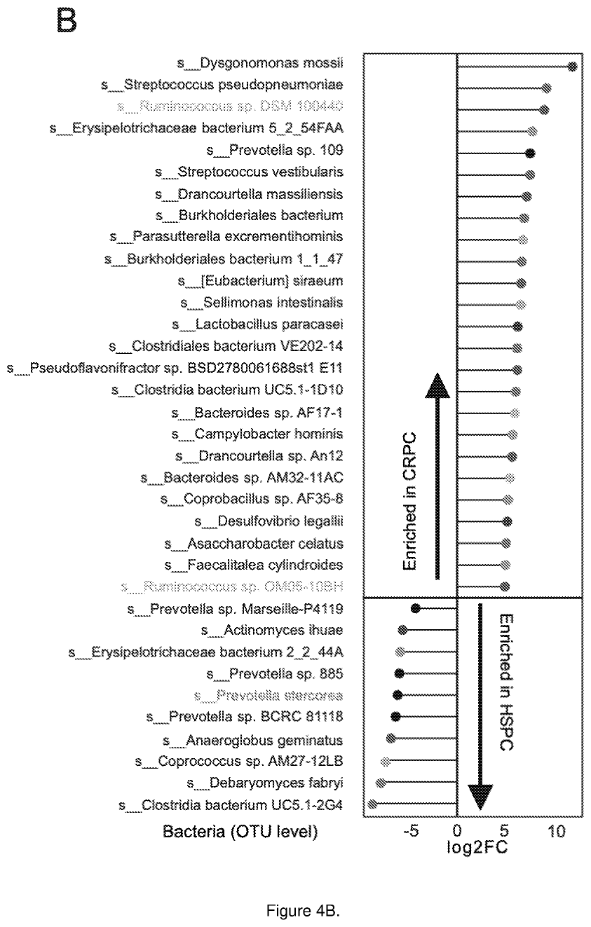

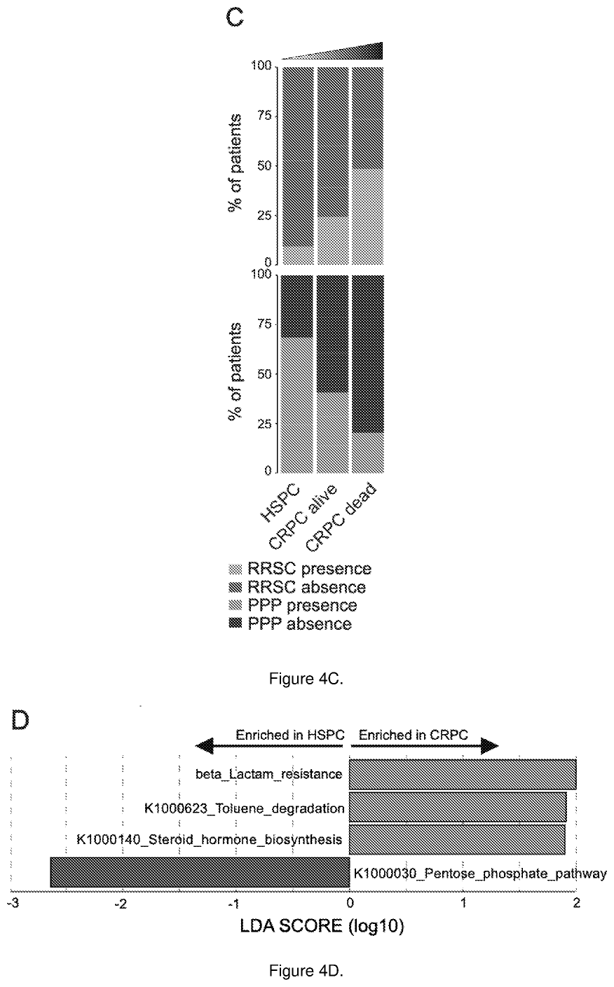

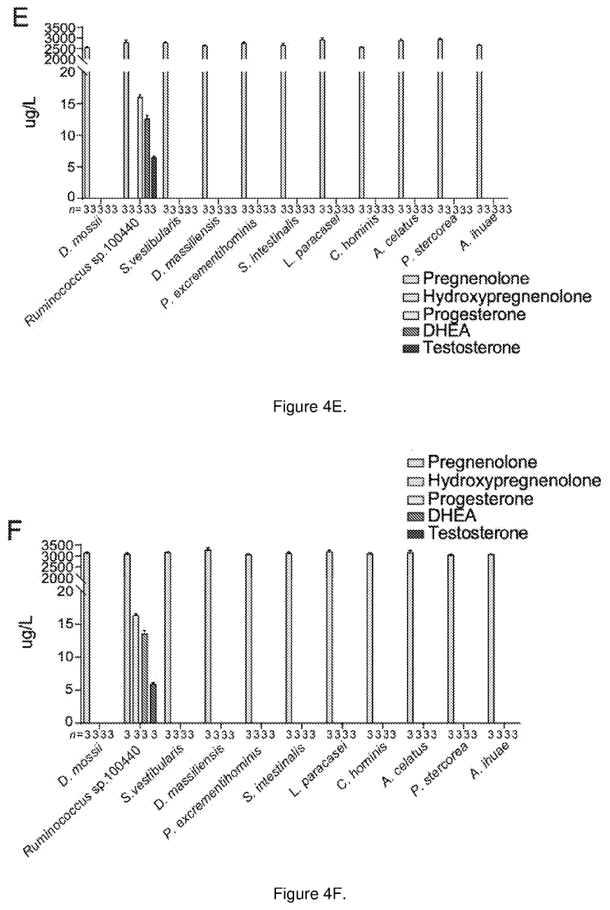

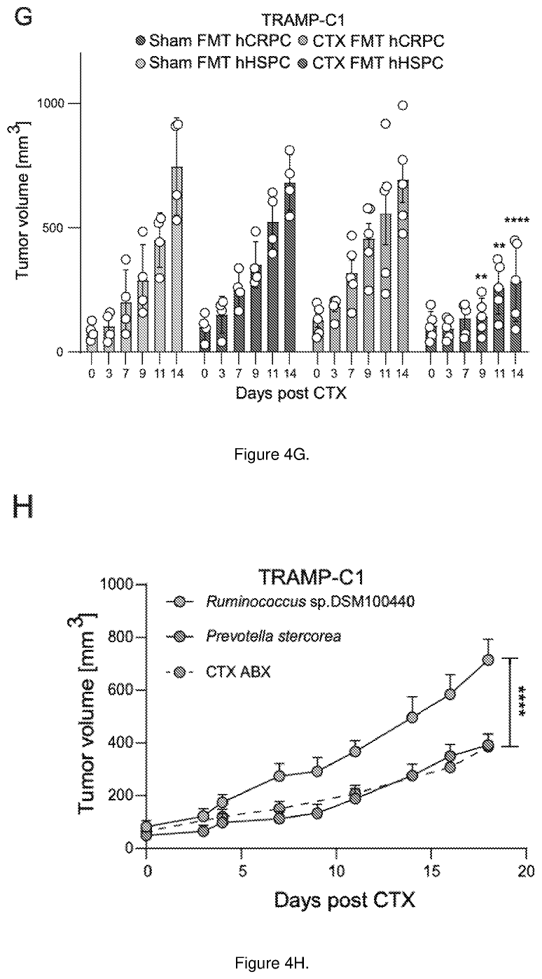

FIG. 4—Metagenomic analysis of human CRPC fecal samples identifies bacterial species that produce androgens and promote castration resistance in gut-humanized mice. Rectal swabs were collected from 19 HSPC and 55 CRPC patients and shotgun whole genome metagenome (WGM) sequencing was performed. (A) Heatmap representing differentially abundant bacterial species (FDR<0.01) between HSPC and CRPC. (B) Waterfall plot showing bacterial species significantly enriched in the two cohorts (species with logFC>4). (C) Frequency of patients with detectable unfavorable (RRSC; Ruminococcus sp. DSM_100440, Ruminococcus sp. OM05_10BH, Streptococcus vestibularis and Clostridiales bacterium VE202_14) or favorable (PPP; Prevotella sp. BCRC_81118, Prevotella sp. Marseille_P4119, Prevotella sp. 885) species according to clinical status (HSPC, CRPC alive or CRPC dead patients). (D) LDA score of top KEGG pathways enriched in HSCP and CRPC of CH cohort. Dysgomonas mossii, Ruminococcus sp. DSM_100440, Streptococcus vestibularis, Drancourtella massiliensis, Parasutterella excrementihominis, Sellimonas intestinalis, Lactobacillus paracasei, Campylobacter hominis, Asaccharobacter celatus, Prevotella stercorea and Actinomyces ihuae were cultured for 48 h in the presence of diverse androgen precursors in anaerobic conditions and analyzed for production of androgens or androgen precursors through LC-MS/MS. Quantification of androgen pathway intermediates in bacterial conditioned media after (E) pregnenolone and (F) hydroxypregnenolone incubation. (G) Tumor volume of TRAMP-C1 mice that were treated with ABX for 7 days before CTX or sham-operation and then with FMT from either hHSPC or hCRPC donors (Sham hHSPC n=4, Sham hCRPC n=4, CTX hCRPC n=5, CTX hCRPC n=5). (H) Tumor volume of TRAMP-C1 mice treated with ABX for 7 days before CTX, then treated via oral gavage with R. sp DSM100440 (n=6), P. stercorea (n=5) or ABX (n=9). Statistical analysis was performed with: (G), (H) two-way ANOVA and Sidak's multiple comparison test. NS, not significant; *P<0.05; **P<0.01; ***P<0.001; ****P<0.0001.

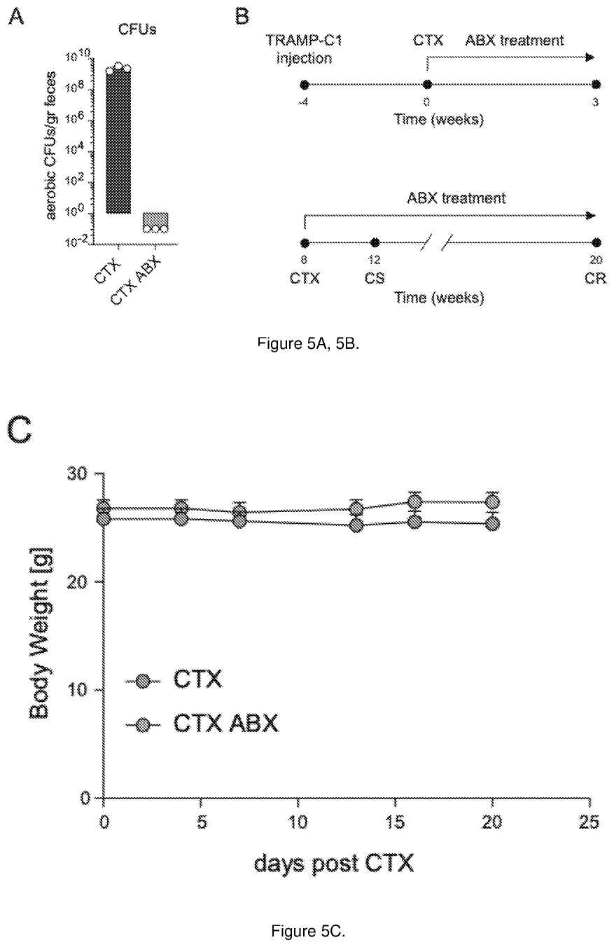

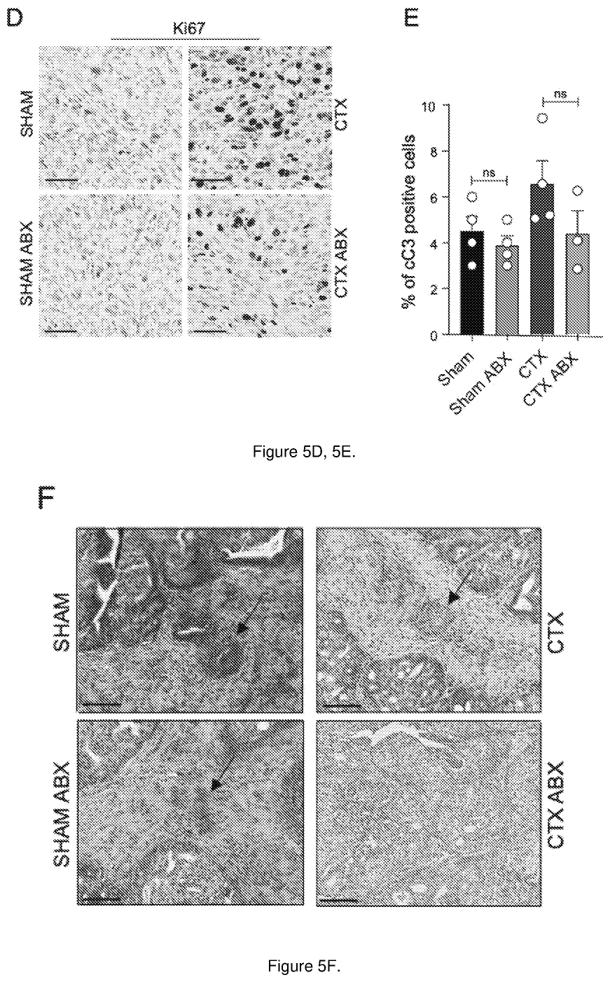

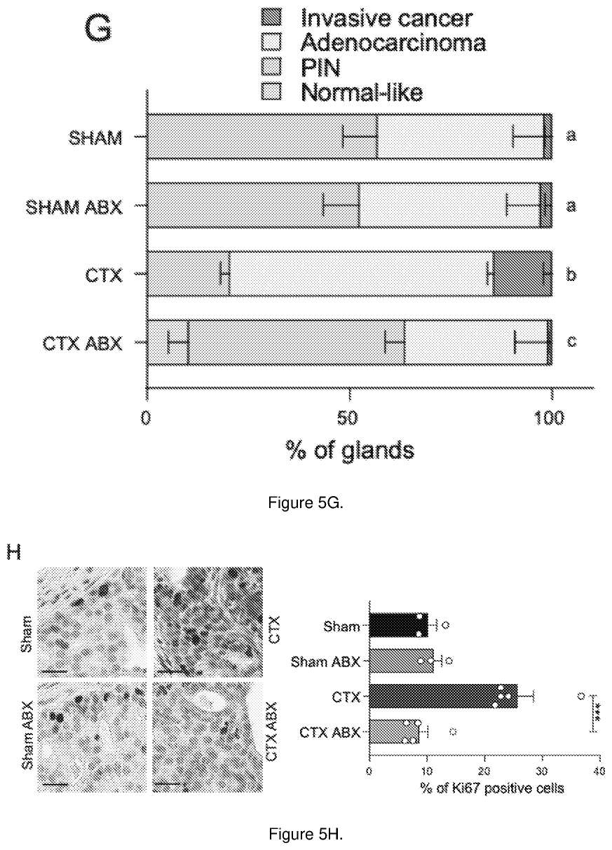

FIG. 5—Efficacy of antibiotic treatment in two mouse models of prostate cancer. (A) Aerobic CFU counts in feces of Pten^{pc-/-} mice CTX (n=3), CTX ABX (n=3). (B) Experimental scheme for CTX and ABX treatment in TRAMP-C1 allografts (top) and Pten^{pc-/-} mice (bottom). (C) Body weight of TRAMP-C1 CTX (n=5) and CTX ABX (n=5) mice. (D) Representative Ki67 images of TRAMP-C1 Sham, Sham ABX, CTX, CTX ABX; scale bar 50 μm (E) Percentage of cC3 positive cells in TRAMP-C1 mice sham (n=4), sham ABX (n=4), CTX (n=4) and CTX ABX (n=3). (F) Representative H&E on AP lobes of Pten^{pc-/-} mice Sham, Sham ABX, CTX or CTX ABX; arrows indicate invasive spots; scale bar 100 μm. (G) Histopathological score of AP lobes of Sham (n=6), Sham ABX (n=5), CTX (n=6) and CTX ABX (n=7) of Pten^{pc-/-} mice at 20 weeks of age. (H) Representative images and quantification of Ki67 positive cells in AP lobes of Sham (n=3), Sham ABX (n=3), CTX (n=5) and CTX ABX (n=5) Pten^{pc-/-} mice at 20 weeks of age; Scale bar 25 μm. (I) TRAMP-C1 cells in FAD were cultured for 72 h in the presence of increasing concentrations of neomycin (N), ampicillin (A), vancomycin (V), metronidazole (M) or combination of the four ABX (NAVM). Cellular viability was measured by crystal violet assay (CTRL n=8, all other conditions n=4). Statistical analysis was performed with: (E), (H) unpaired two-sided Student's t-test; (G) one-way ANOVA (p<0.01). NS, not significant; *P<0.05; **P<0.01; ***P<0.001; ****P<0.0001.

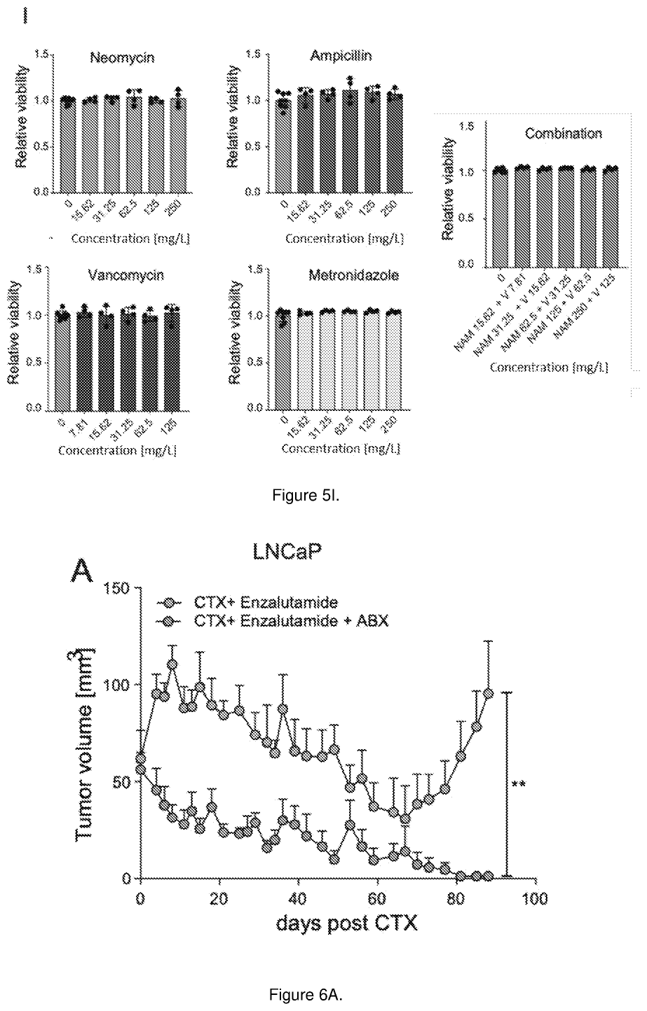

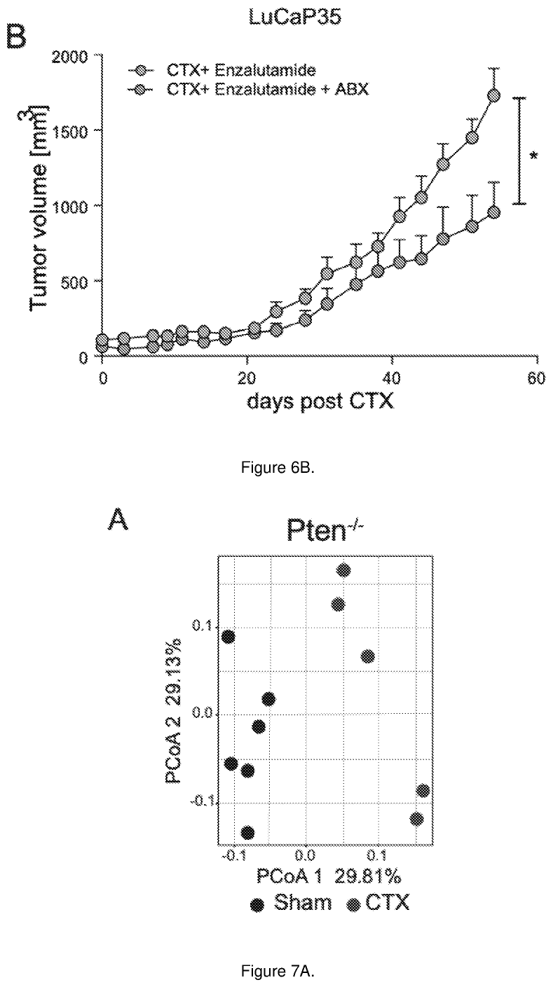

FIG. 6—ABX treatment is effective in enzalutamide-treated castration sensitive mouse models. NRG mice were castrated when tumor was palpable and either treated with Enzalutamide or Enzalutamide+ABX. (A) Tumor volume of LNCaP allograft (CTX+Enzalutamide n=6, CTX+Enzalutamide+ABX n=6). (B) Tumor volume of LuCaP35 allograft (CTX+Enzalutamide n=6, CTX+Enzalutamide+ABX n=5). Statistical analysis was performed in (A), (B) with unpaired two-sided Student's t-test; *P<0.05; **P<0.01.

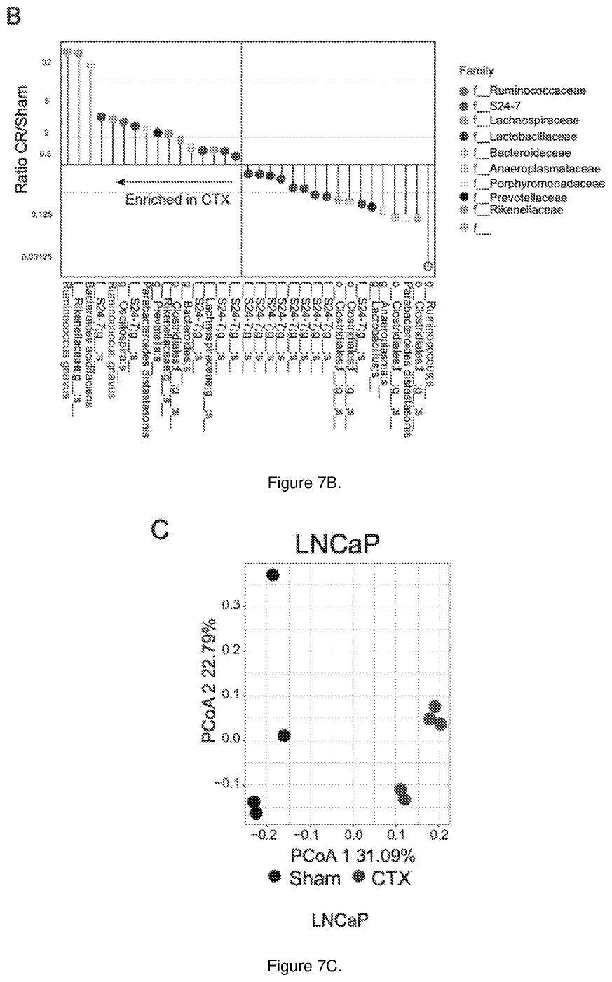

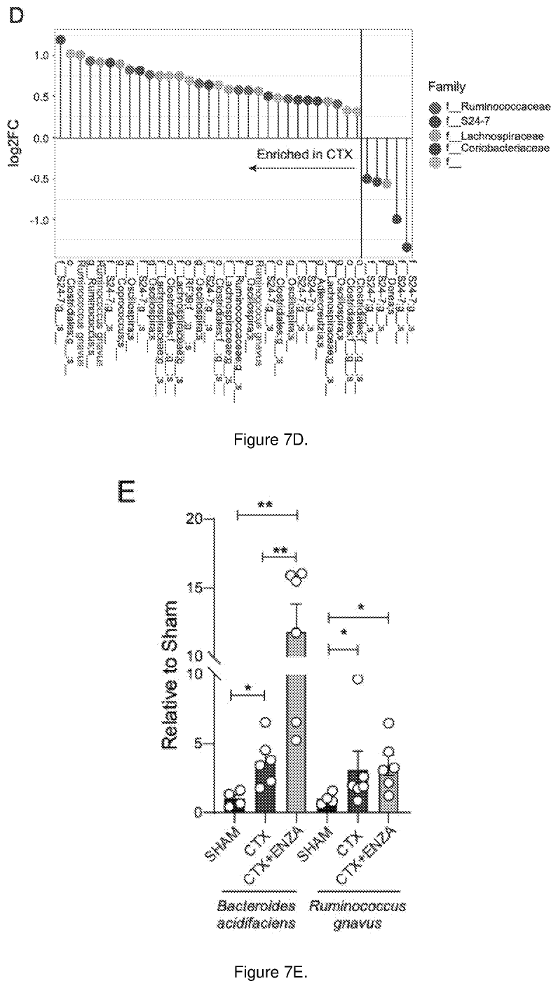

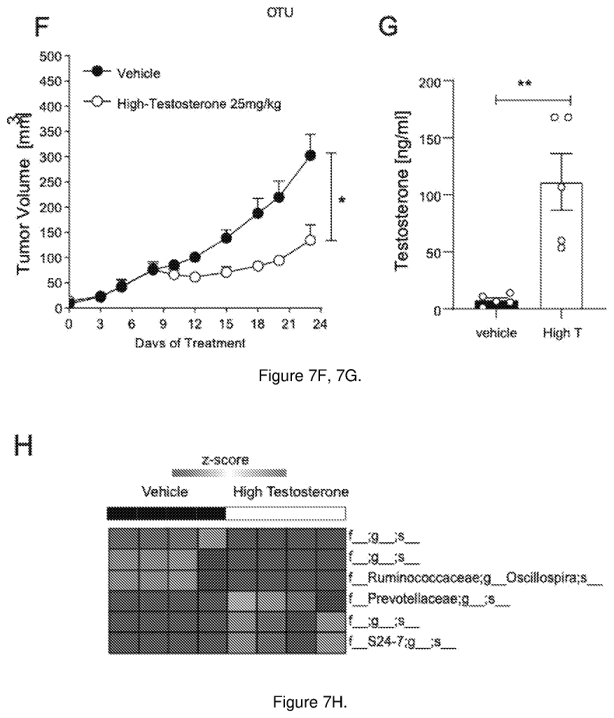

FIG. 7—Pten^{pc-/-} and LNCaP castrated mice harbor a peculiar gut microbiota. The composition of the CS and CR microbiota was analyzed through 16S rDNA sequencing in feces of Pten^{pc-/-} and LNCaP mice in Sham and CR phase. (A) Principal component analysis (PCoA) and (B) waterfall plot showing the abundance of the significantly different species (FDR<0.05) as a ratio CR/Sham in Pten^{pc-/-} mice (Sham n=6, CTX n=5). (C) Principal component analysis (PCoA) and (D) waterfall plot showing the abundance, expressed as log fold change, of the different species significantly enriched (FDR<0.05) in Sham (n=4) and CTX (n=5) cohort in LNCaP mice. (E) qPCR showing the abundance of R. gnavus and B. acidifaciens in feces of LNCaP mice that were either sham-operated (n=4), CTX (n=6), CTX+enzalutamide (n=6) at 85 days. TRAMP-C1 mouse models were treated with either vehicle or high dose of testosterone as per bipolar androgen therapy. (F) Tumor volume (vehicle n=5, high testosterone n=5). (G) Serum testosterone levels (vehicle n=5, high-testosterone n=5). (H) Heatmap representing differentially abundant bacteria (FDR<0.05) between mice injected with vehicle or high testosterone-dose (vehicle n=4, high-testosterone n=4). Statistical analysis was performed in (E), (G) with unpaired two-sided Student's t-test; *P<0.05; **P<0.01.

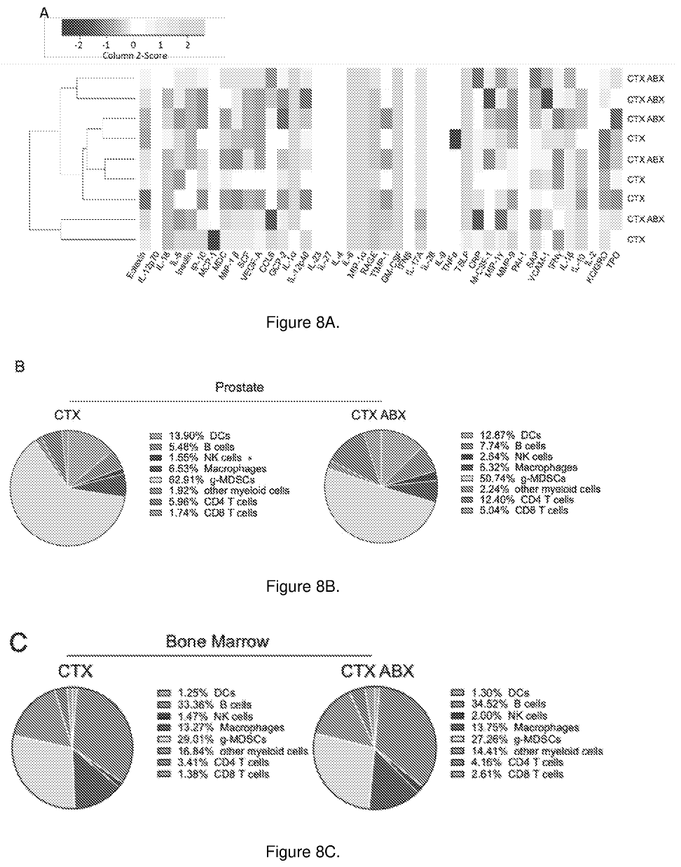

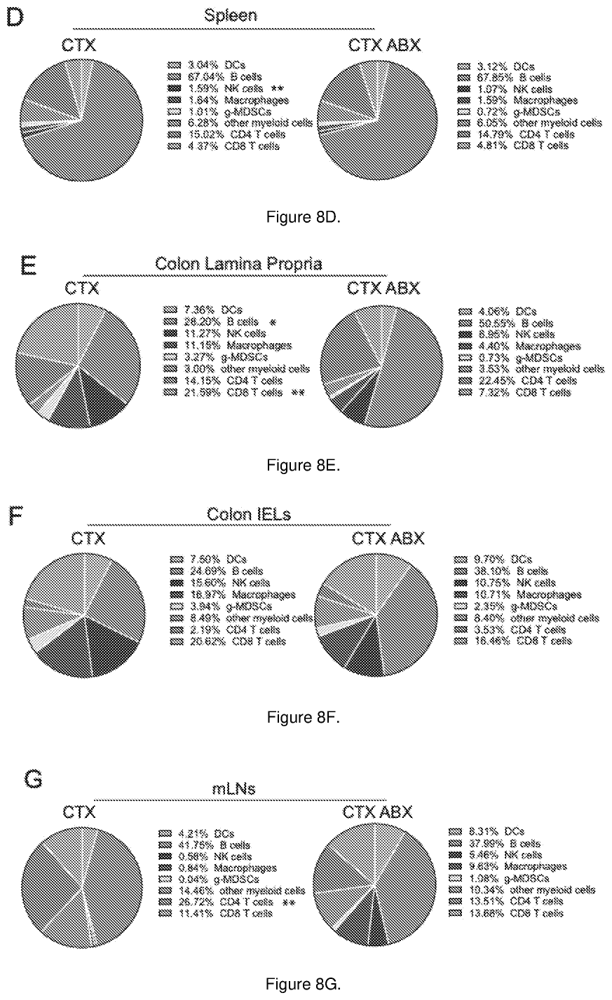

FIG. 8—ABX treatment does not impact on the systemic and tumor immune response in Pten^{pc-/-} mice. Pten^{pc-/-} mice were CTX and either treated with ABX (n=5) or not (n=4) until 20 weeks of age. (A) Analysis of cytokine abundance in the serum. Multiparametric flow cytometry analysis of the immune compartment was performed CTX (n=6) and CTX ABX (n=5) on (B) Prostate, (C) Bone marrow, (D) Spleen, (E) Colon lamina propria, (F) Colon intraepithelial lymphocytes (IELs), (G) mesenteric lymph nodes (mLNs). Statistical analysis in: (B), (C), (D), (E), (F), (G) was performed with unpaired two-sided Student's t-test. NS, not significant; *P<0.05; **P<0.01; ***P<0.001; ****P<0.0001.

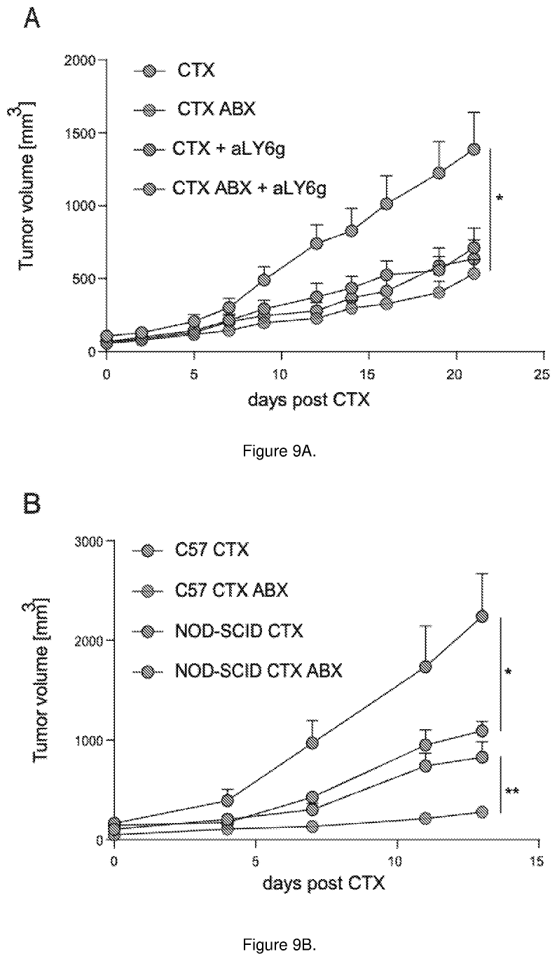

FIG. 9—Microbiota ablation is also effective in immunodeficient prostate cancer models. TRAMP-C1 allograft mice were CTX then either left untreated (n=9), treated with ABX (n=9) or with (A) αLy6G alone (n=8) or in combination with ABX (n=8). NOD-SCID or C57BL6/N mice were challenged s.c. with 2.5×10⁶ TRAMP-C1 cells. After CTX (B) mice were left untreated (n=5) or treated with ABX (n=5). Statistical analysis was performed with: (A-B) two-way ANOVA and Sidak's multiple comparison test; NS, not significant; *P<0.05; **P<0.01; ***P<0.001; ****P<0.0001.

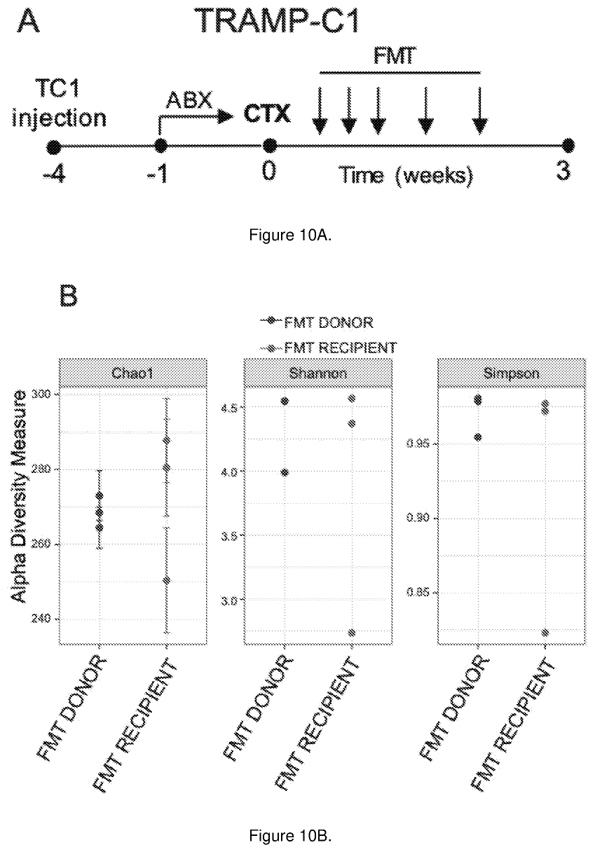

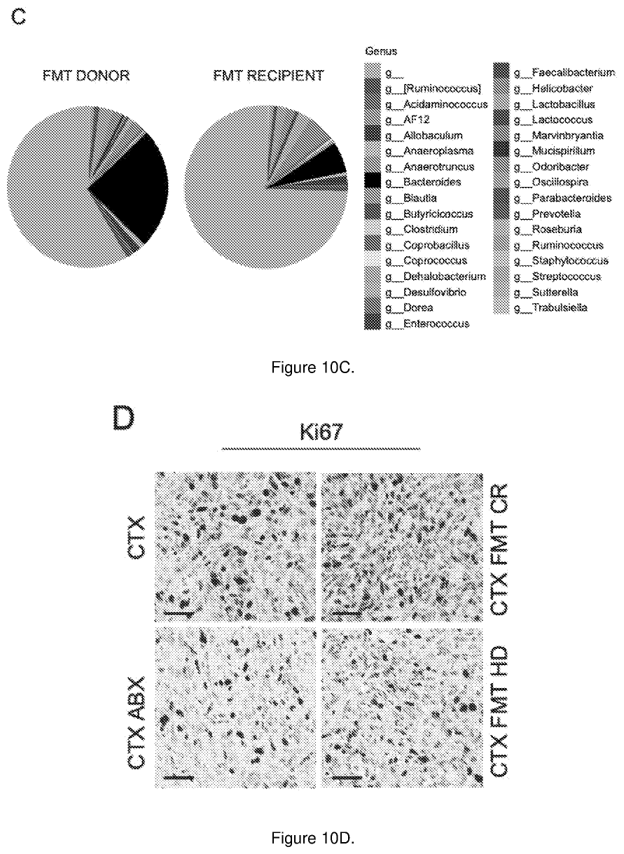

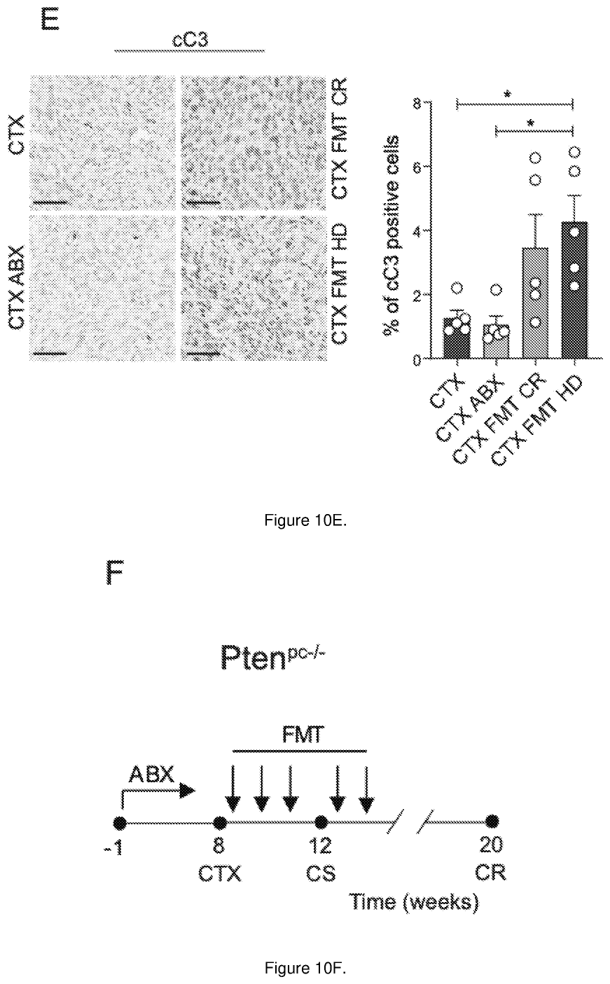

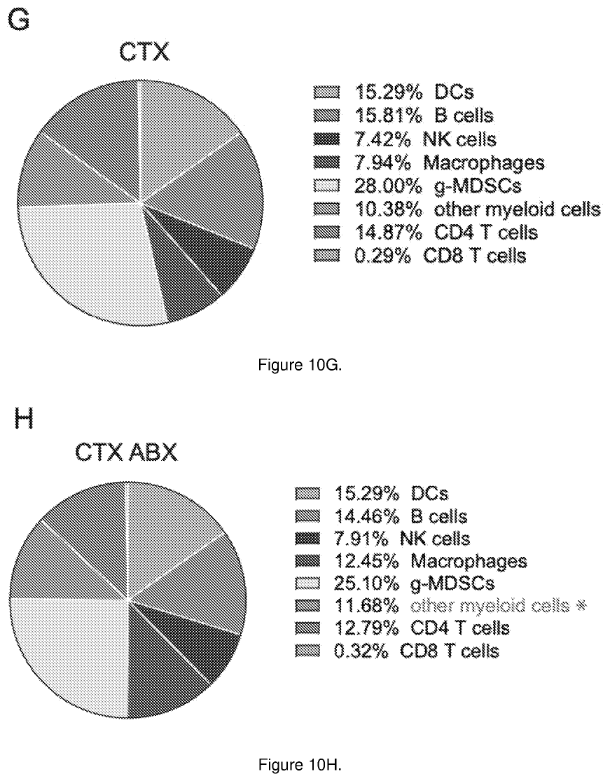

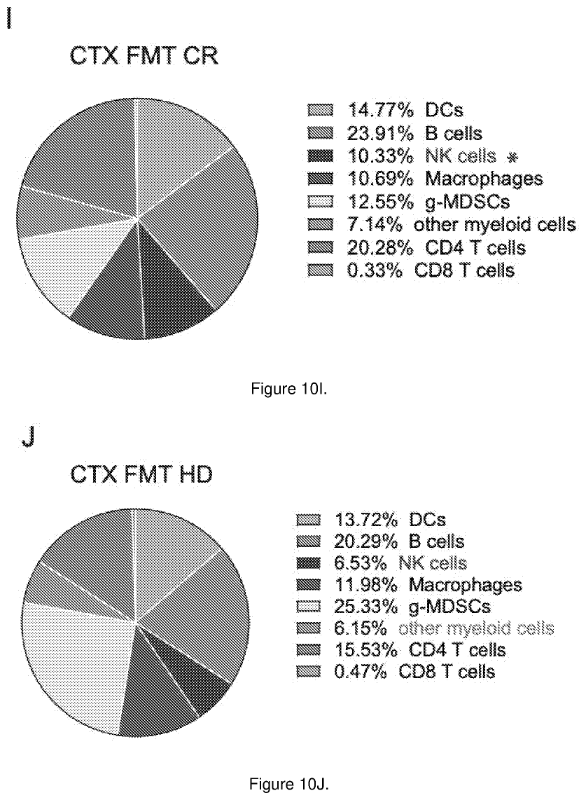

FIG. 10—FMT from CR but not HD results in delayed prostate tumor growth. C57BL6/N mice were challenged s.c. with TRAMP-C1 cells and treated with ABX for 7 days before CTX, then either left untreated, treated with ABX or received FMT from healthy donors (HD FMT) or CR donors. (A) Experimental scheme of FMT treatment in TRAMP-C1 mice. The composition of the gut microbiota comparing donor mice for FMT (FMT donor, n=3) and the mice reconstituted with the feces of FMT donor in CR Pten^{pc-/-} mice (FMT recipient, n=3) was analyzed through 16S rDNA sequencing. (B) Diversity indexes of gut microbiota composition and Pie chart representing the gut microbiota composition at (C) genus level comparing FMT donor and FMT recipient. (D) Representative images of Ki67 IHC staining; scale bar 50 μm. (E) Representative IHC images (left) and quantification of the percentage of cC3 positive cells (right) in TRAMP-C1 CTX (n=5), CTX ABX (n=5), CTX FMT CR (n=5), CTX FMT HD (n=5) mice; scale bar 50 μm. (F) Experimental scheme of FMT experiment in Pten^{pc-/-} mice. Multiparametric flow cytometry analysis was performed on TRAMPC-C1 allograft tumors of (G) CTX (n=5), (H) CTX ABX (n=5), (1) CTX FMT CR (n=5), (J) CTX FMT WT (n=6) mice. Statistical analysis was performed with: (E) one-way ANOVA and Tukey's multiple comparison test; (G), (H), (I), (J) two-way ANOVA and Tukey's multiple comparison test; NS, not significant; *P<0.05.

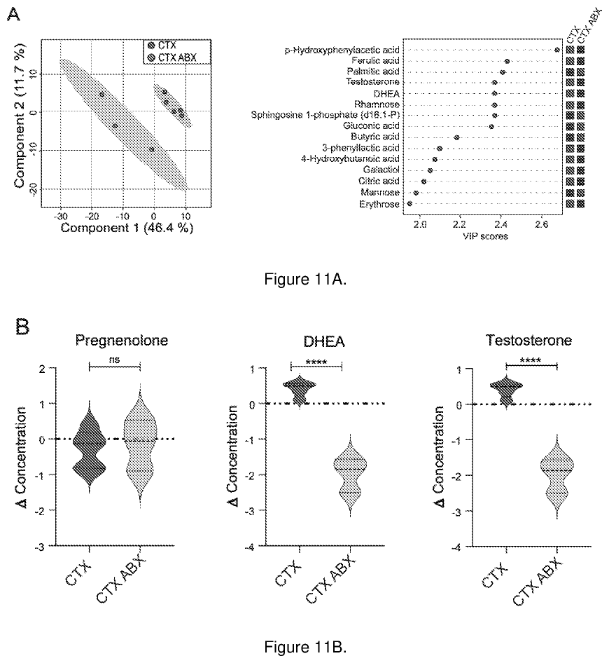

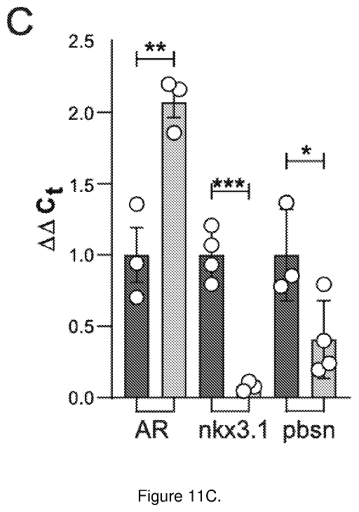

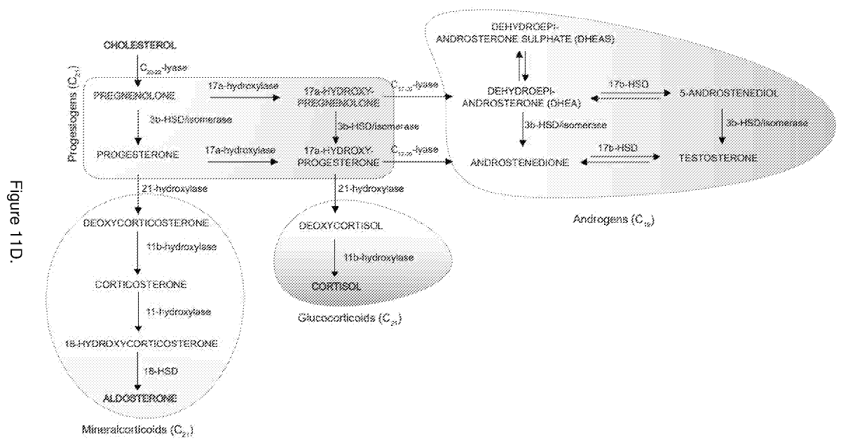

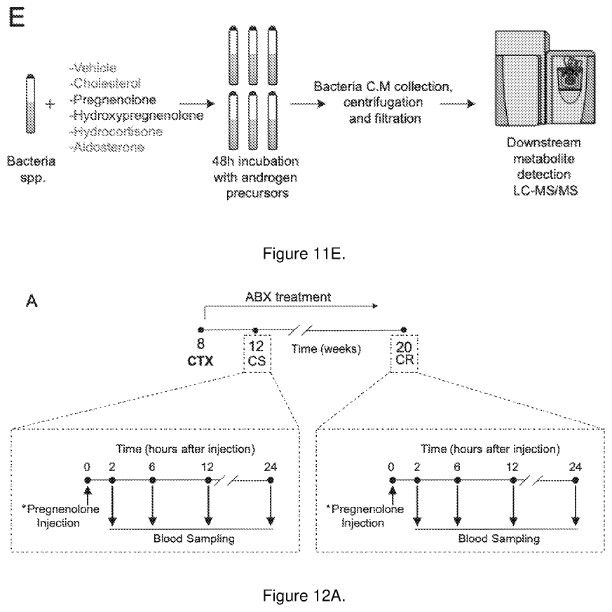

FIG. 11—Gut microbiota ablation decreases circulating androgen levels in castrated hosts. Untargeted metabolomic analysis was performed in sera of Pten^{pc-/-} CTX and CTX ABX mice at 20 weeks of age. (A) Left: Principal component analysis (PCoA) of untargeted metabolomic analysis on serum of Pten^{pc-/-} CTX (n=5) or CTX ABX (n=3) mice at 20 weeks of age. Right: metabolites differentially abundant in CTX and CTX ABX Pten^{pc-/-} mice (VIP score >2). (B) Relative quantification of pregnenolone (left), DHEA (middle) and testosterone (right); (CTX n=5, CTX ABX n=3). (C) Reverse transcription-quantitative PCR (qRT-PCR) analyses of AR (CTX n=3, CTX ABX n=3), Nkx3.1 (CTX n=4, CTX ABX n=3) and probasin (pbsn) (CTX n=3, CTX ABX n=4) in AP lobes from Pten^{pc-/-} CTX and CTX ABX mice at 20 weeks of age. (D) Biosynthetic pathway of androgens, glucocorticoids and mineralcorticoids. Intermediate metabolites used in metabolite conversion experiments are represented in bold; green ones represent the precursor metabolized by the bacteria while red ones represent those tested but not metabolized. (E) Experimental scheme of bacterial cultured for 48 h in the presence of the indicated androgen precursors in anaerobic conditions and analyzed for production of androgens or androgen precursors through LC-MS/MS.

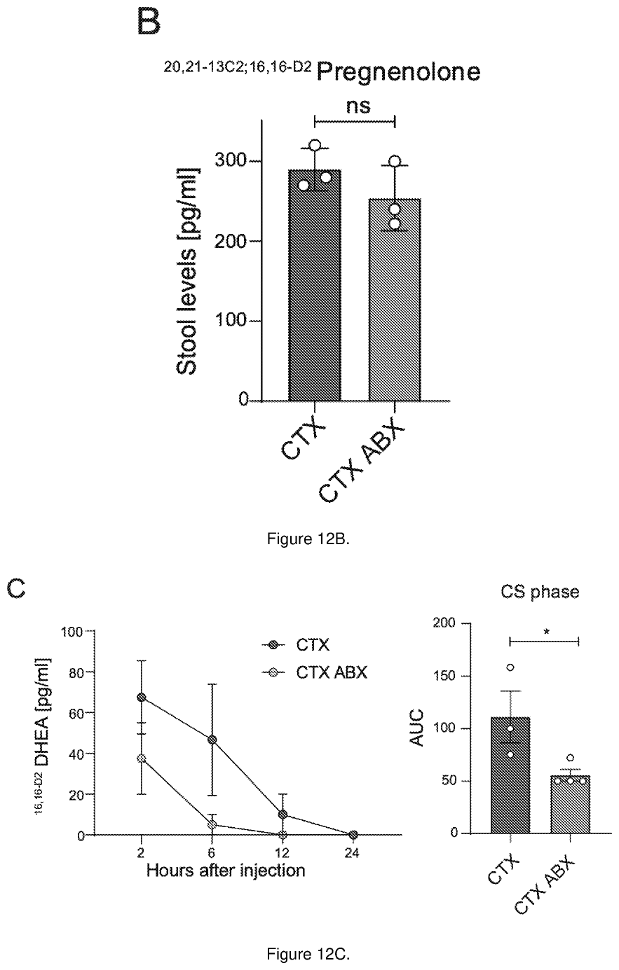

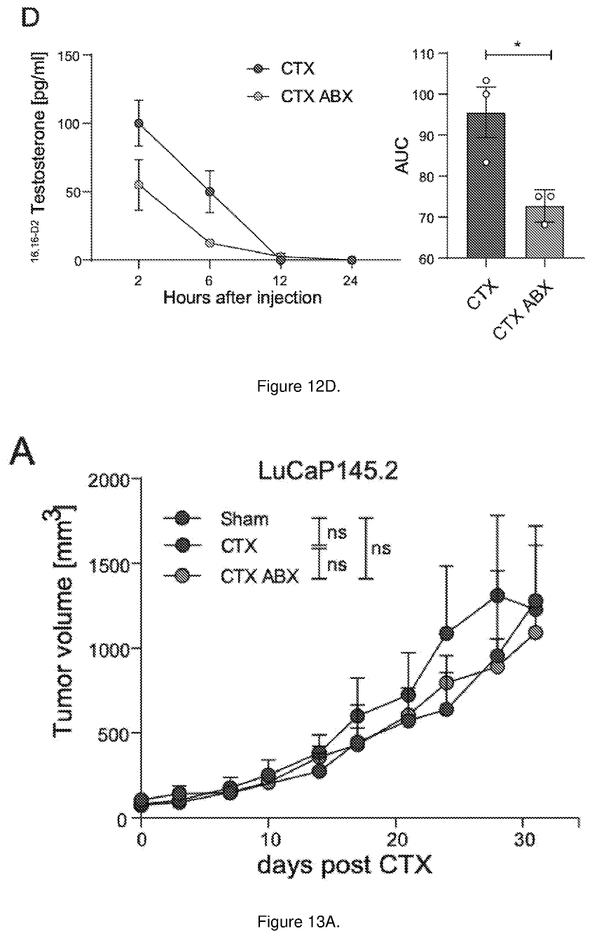

FIG. 12—The gut microbiota participates in androgen biosynthesis in castrated hosts. Pten^{pc-/-} mice treated or not with ABX were injected i.v. with 75 ng of ^{D}pregnenolone. (A) Experimental scheme: ^{D}pregnenolone was injected in Pten^{pc-/-} mice at 12 and 20 weeks of age. Serum was sampled at 2, 6, 12 and 24 h after injection and analysed for the presence of deuterated metabolites. (B) Fecal levels of ^{D}pregenolone in CTX (n=3) e CTX ABX (n=3) mice 6 h after injection. Time-course quantification and AUC of C) ^{D}DHEA (CTX n=3; CTX ABX n=4) and (D) ^{D}testosterone (CTX n=3; CTX ABX n=3) in sera of mice at 12 weeks of age treated as described in A). Statistical analysis was performed with: (B), (C), (D) unpaired two-sided Student's t-test. NS, not significant; *P<0.05.

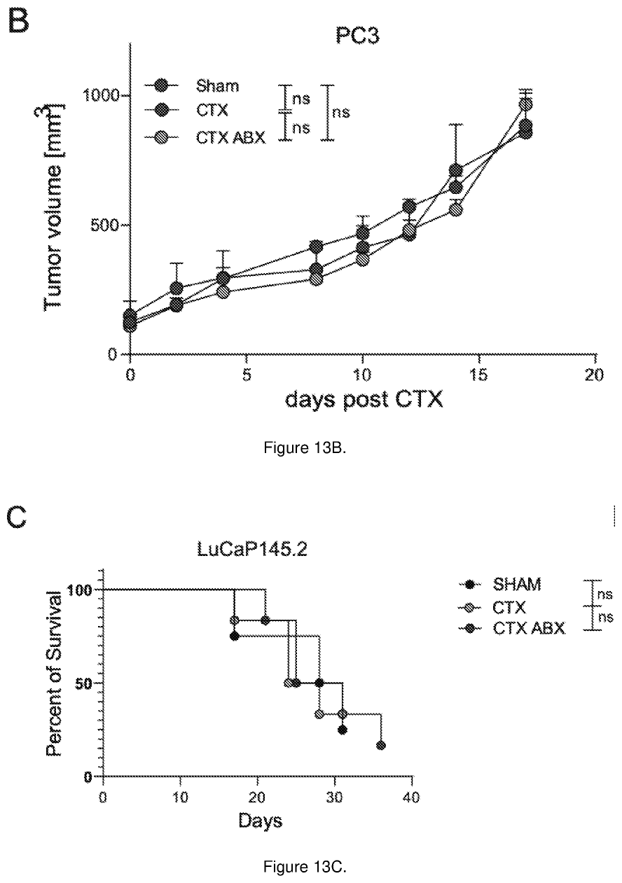

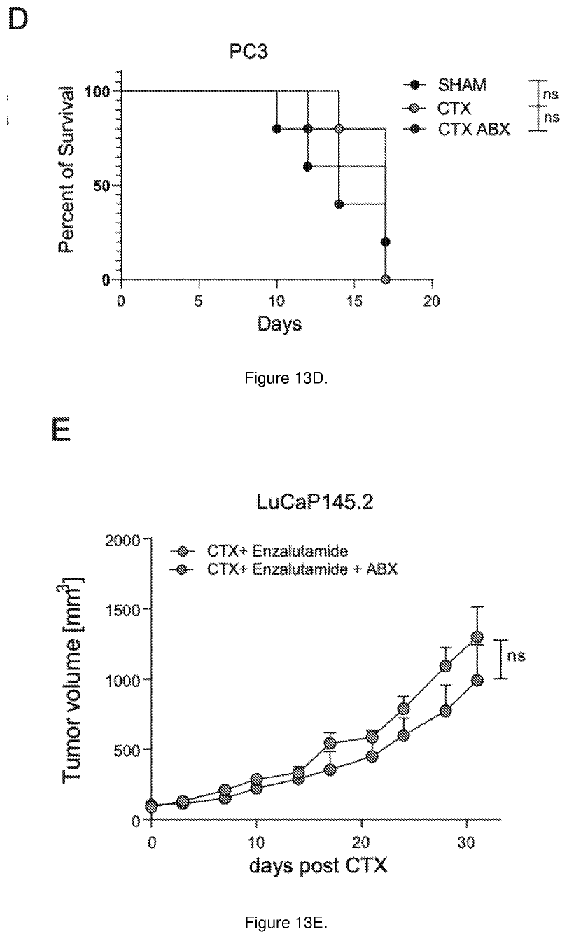

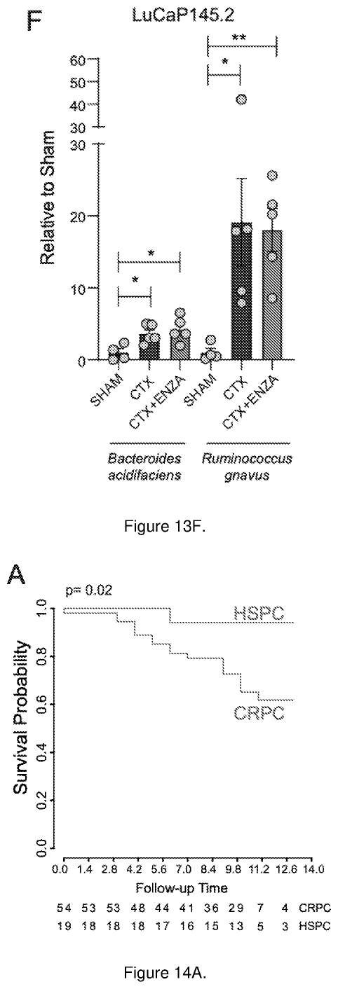

FIG. 13—Gut microbiota ablation is non-effective in androgen resistant models. NRG mice harboring LuCaP145.2 (A) or PC3 (B) xenograft tumors were either castrated or sham-operated when the tumor was palpable and either left untreated or treated with ABX. (A) Tumor volume and (C) survival curve of LuCaP145 allograft (model insensitive to CTX); Sham (n=4), CTX (n=6), CTX ABX (n=6). (B) Tumor volume and (D) survival curve of PC3 allograft (model insensitive to CTX); Sham (n=5), CTX (n=5), CTX ABX (n=5). (E) Tumor volume of NRG mice harboring LuCaP145.2 xenograft tumors that were castrated and either treated with enzalutamide (n=5) or enzalutamide+ABX (n=5). (F) qPCR showing the abundance of R. gnavus and B. acidifaciens in feces of LuCaP145.2 mice that were either sham-operated (n=4), CTX (n=6), CTX+enzalutamide (n=6) at 35 days. Statistical analysis was performed with: (A), (B), (E) two-way ANOVA and Sidak's multiple comparison test; (C), (D), Mantel Cox test, (F) unpaired two-sided Student's t-test. NS, not significant, *P<0.05; **P<0.01.

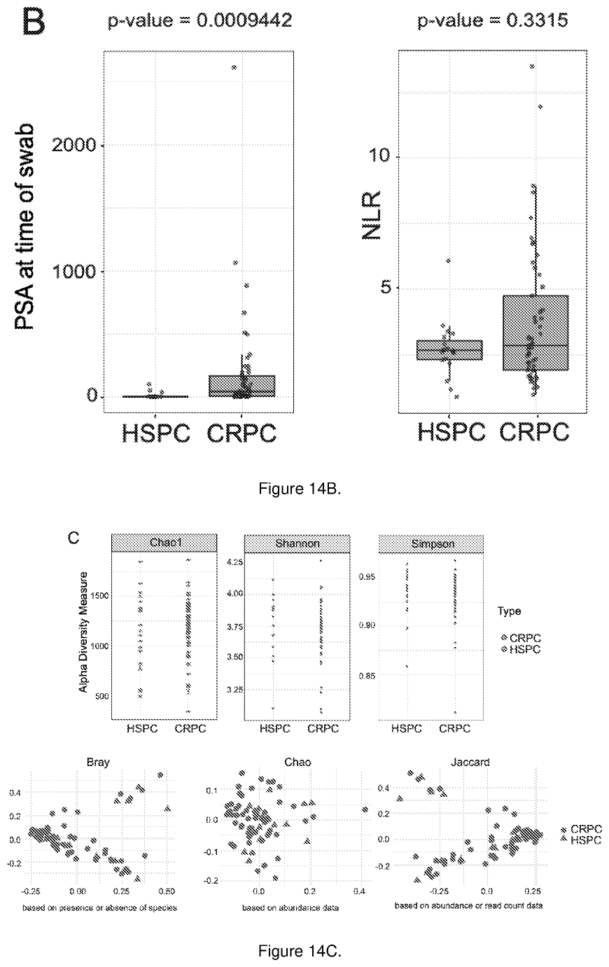

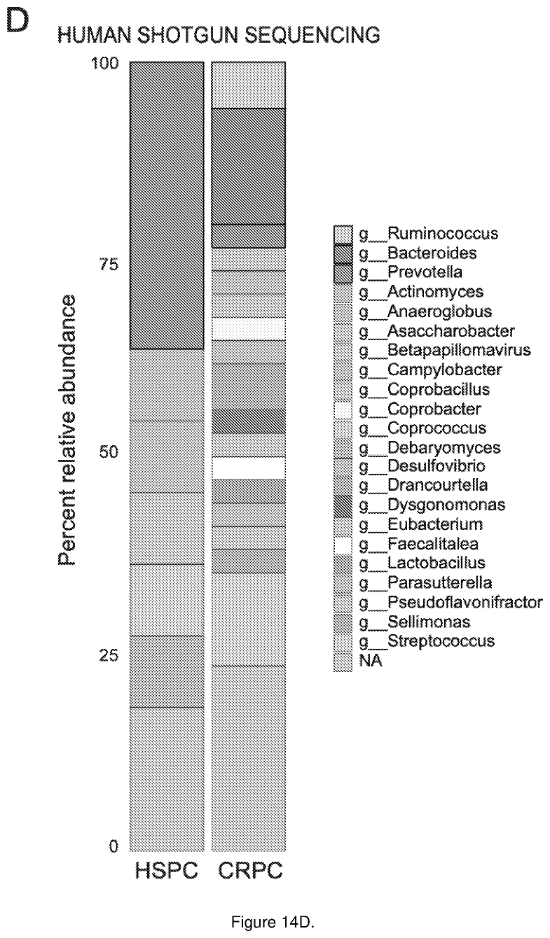

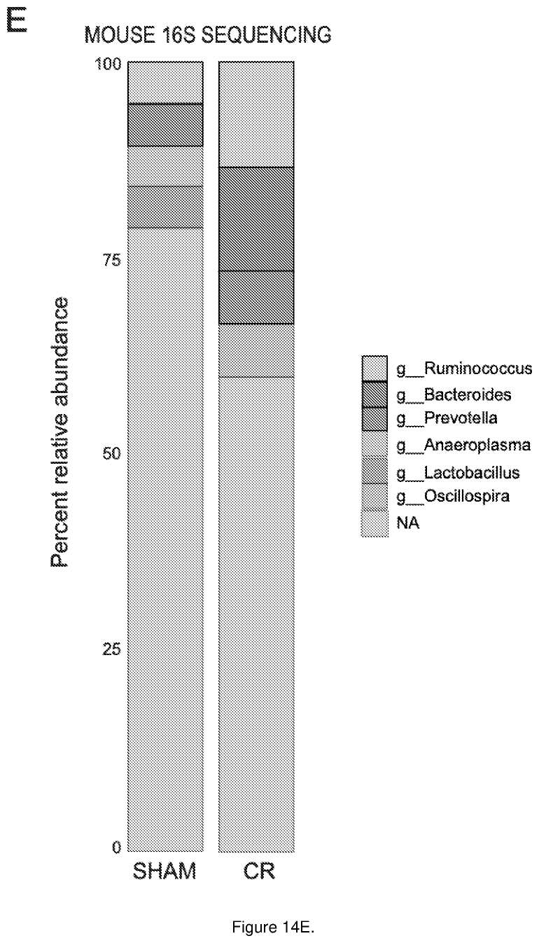

FIG. 14—Fecal microbiota of hCRPC patients has peculiar composition. (A) Overall survival of HSPC and CRPC patients analyzed in the study. (B) Prostate specific antigen (PSA) (left) and neutrophil lymphocyte ratio (NLR) (left) at time of fecal swab collection in HSPC and CRPC cohorts. (C) The gut microbiomes of HSPC and CRPC were tested for alpha-diversity (top) and beta-diversity (bottom) including Chao1, Shannon, Simpson, Bray, Chao and Jaccard indices. Comparison of the fecal microbiota of (D) HSPC and CRPC patients and (E) sham and CR mice. Graphs show the percentage of genera enriched in each group. Statistical analysis was performed with: (A) log-rank (Mantel-Cox) analysis; (B) nonparametric Mann-Whitney two-sided t-test.

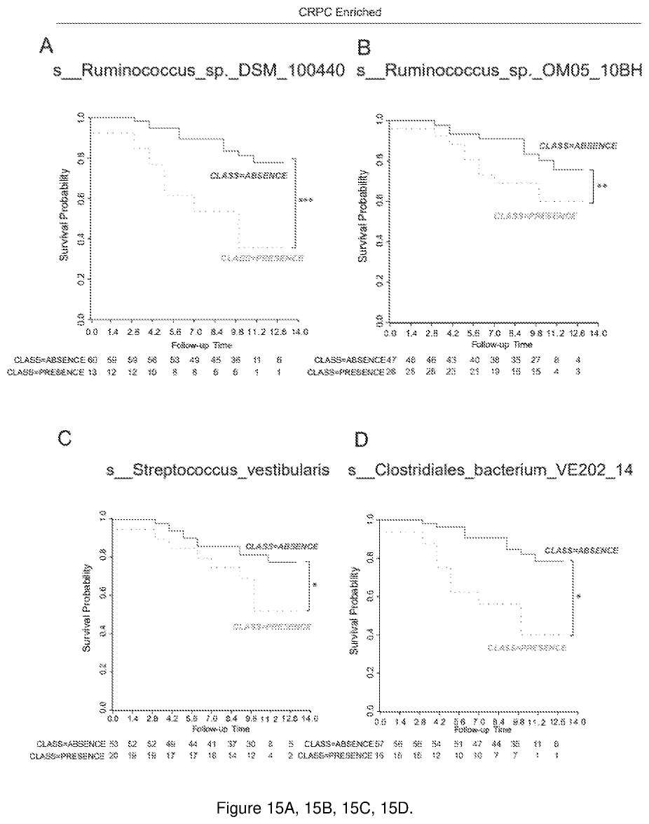

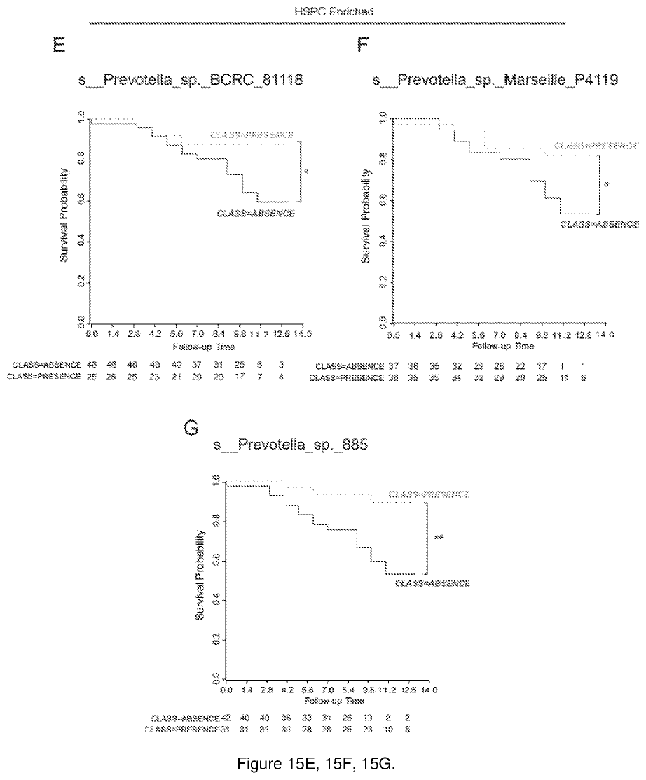

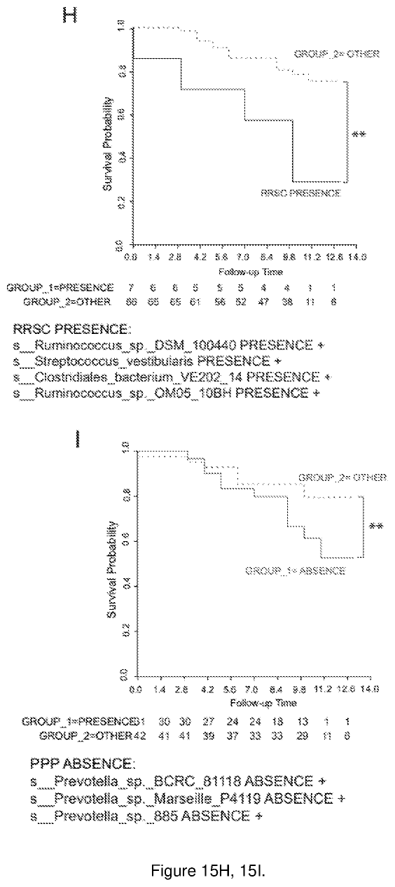

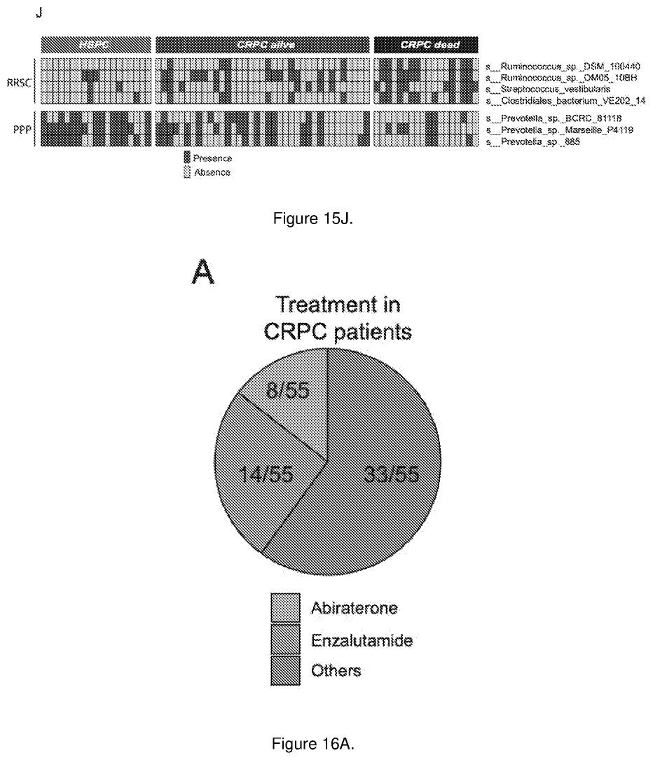

FIG. 15—Gut bacterial fingerprint predicts prostate cancer patients' prognosis. Overall survival analyses stratifying patients based on presence or absence in the fecal microbiota of the following species: (A), Ruminococcus sp. DSM_100440, (B) Ruminococcus sp. OM05_10BH (C) Streptococcus vestibularis, (D) Clostridiales bacterium VE202_14, (E) Prevotella sp. BCRC_81118, (F) Prevotella sp. Marseille_P4119, (G) Prevotella sp. 885. Kaplan-Meier survival curves stratifying patients in high and low risk groups based on the presence of: (H) “high risk” signature (RRSC): simultaneous presence of Ruminococcus sp. DSM_100440, Ruminococcus sp. OM05_10BH, Streptococcus vestibularis, Clostridiales bacterium VE202_14 (red line, patients were the four species were not simultaneously present: dashed-blue line); (1) “low risk” signature (PPP): simultaneous absence of the three species Prevotella sp. BCRC_81118, Prevotella sp. Marseille_P4119, Prevotella sp. 885 (green line; patients were at least one of the three species was present: dashed-blue line). (J) Heatmap showing the presence or absence of favorable (PPP; Prevotella sp. BCRC_81118, Prevotella sp. Marseille_P4119, Prevotella sp. 885) and unfavorable (RRSC; Ruminococcus sp. DSM_100440, Ruminococcus sp. OM05_10BH, Streptococcus vestibularis and Clostridiales bacterium VE202_14) species in the fecal microbiota of patients, classified according to clinical status (HSPC, CRPC alive or CRPC dead patients). Presence or absence of bacteria in each patient was performed using decostand R function (see methods). Statistical analysis was performed with (A-I) Mantel-Cox log-rank analysis.

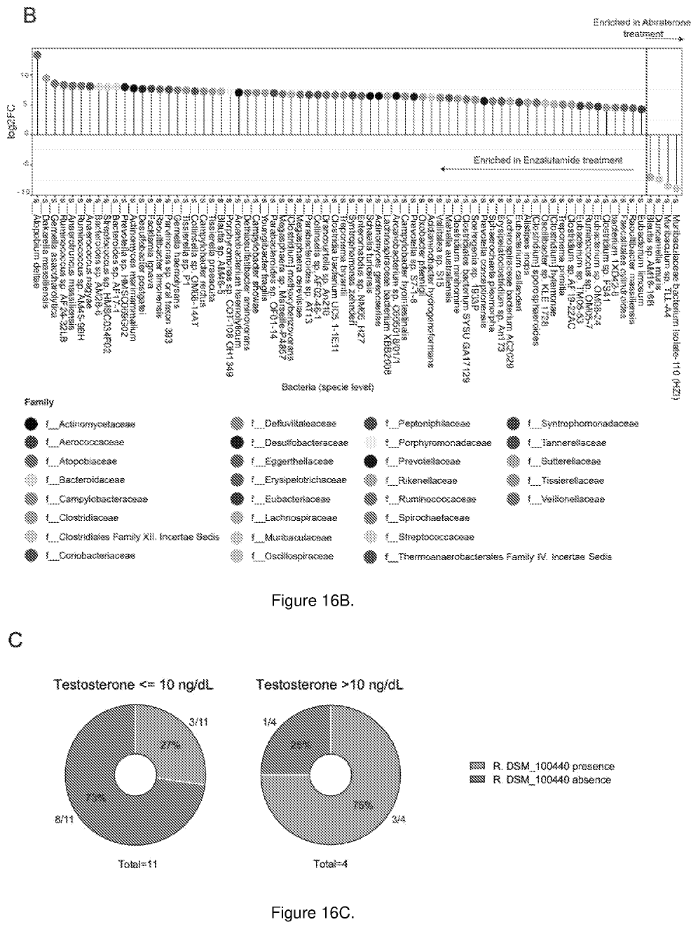

FIG. 16—Different ADT regimens alter gut microbiota composition. (A) Pie chart representing the number of CRPC patients receiving Enzalutamide, Abiraterone or other treatment (Apalutamide, Docetaxel, Cabazitaxel or no active treatment) at the moment of the rectal swab. (B) Waterfall plot showing bacterial species significantly enriched in Enzalutamide- or Abiraterone-treated cohorts (FDR<0.01). (C) Patients treated with enzalutamide (n=14) and apalutamide (n=1) were stratified according to the circulating testosterone level (≤10 ng/dL or >10 ng/dL). Pie charts show, within each group, the fraction of patients having Ruminococcus DSM_10044 in their gut microbiota.

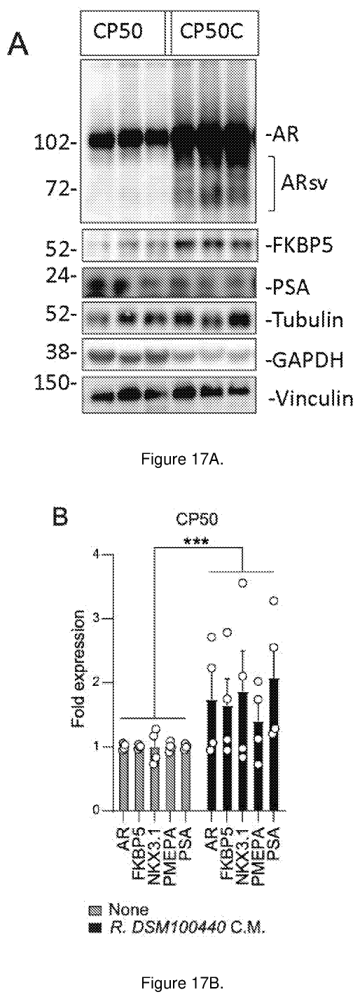

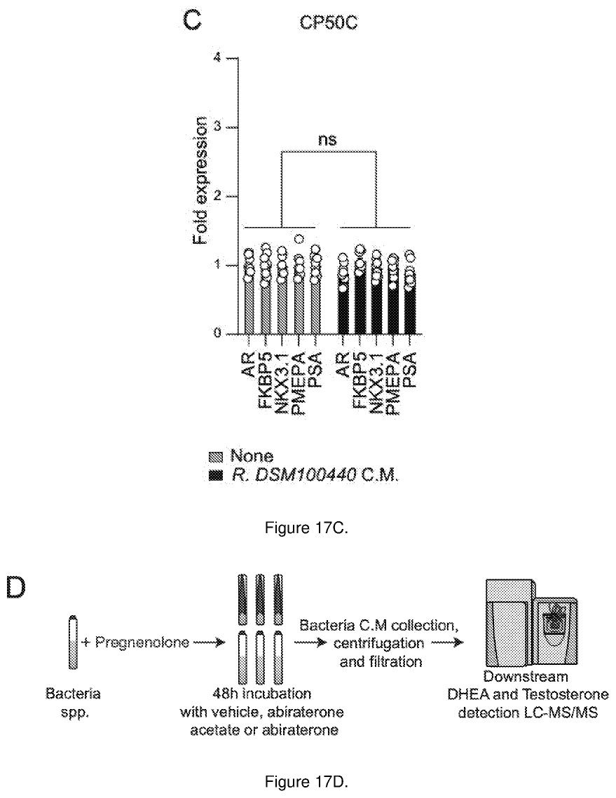

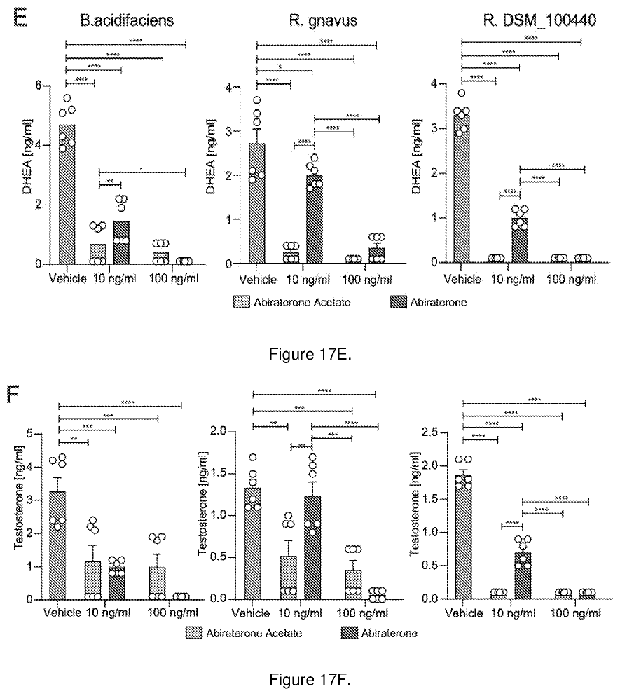

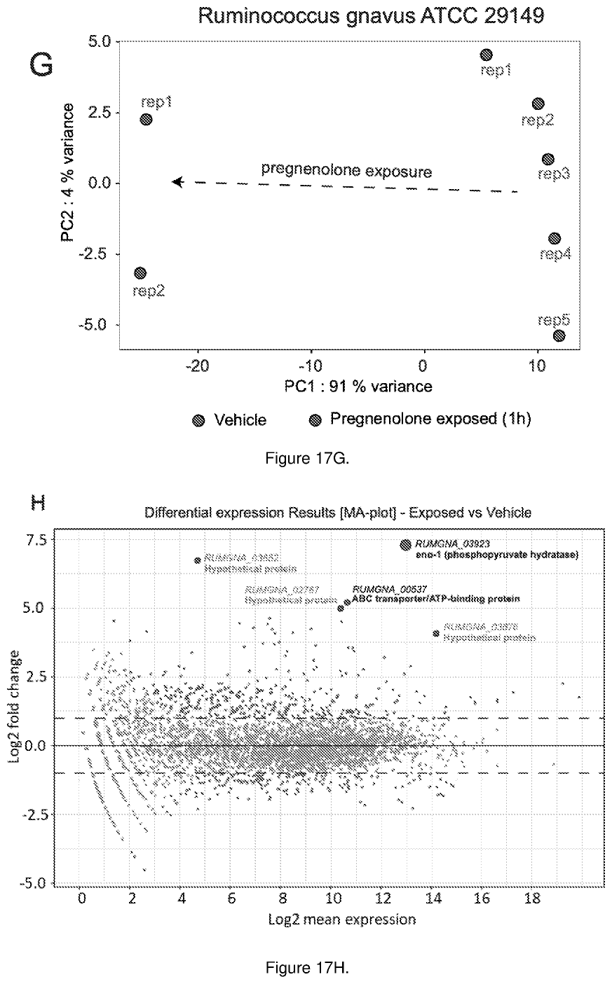

FIG. 17—Abiraterone selectively inhibits bacterial androgen biosynthetic pathway. (A) Western blot of AR, ARsv, FKBP5, PSA, Tubulin, GAPDH and Vinculin in CP50 and CP50C PDOs. Quantitative PCR with reverse transcription (qRT-PCR) of AR, FKBP5, NKX3.1, PMEPA and PSA in (B) CP50 and (C) CP50C PDOs either treated with vehicle or R. sp. DSM_100440 C. M. B. acidifaciens, R. gnavus and Ruminococcus sp. DSM_100440 were incubated in anaerobic condition with pregenolone and either vehicle, abiraterone acetate or abiraterone and the levels of DHEA and Testosterone in the bacterial CM was measured by LC-MS/MS. (D) Experimental scheme. (E) DHEA concentration in bacterial medium. (F) Testosterone concentration in bacterial medium; n=6 for all the conditions. RNA sequencing of Ruminococcus gnavus ATCC 29149 after exposure to pregnenolone. (G) Principal component analysis (PCoA) showing the effect of pregnenolone exposure on gene expression profile. (H) MA-plot showing differentially expressed genes between vehicle and pregnenolone-exposed bacteria. (1) Heatmap showing the percentages of homology and identity between human CYP17A1 (NP_000093.1) and genes up-regulated with log 2 fold change >3.5. Statistical analysis was performed with: (B) (C) unpaired two-sided Student's t-test; (E) (F) one-way ANOVA and Tukey's multiple comparison test; NS, not significant; *P<0.05; **P<0.01; ***P<0.001; ****P<0.0001.

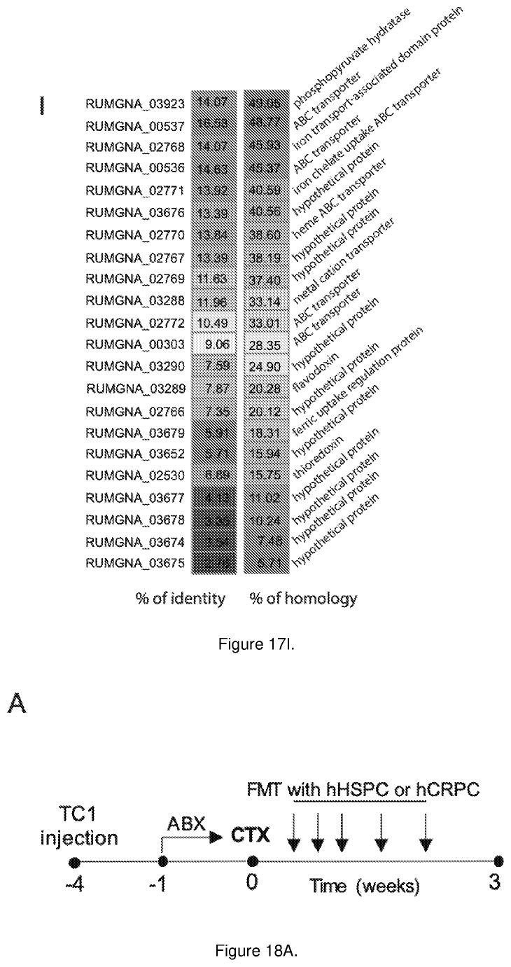

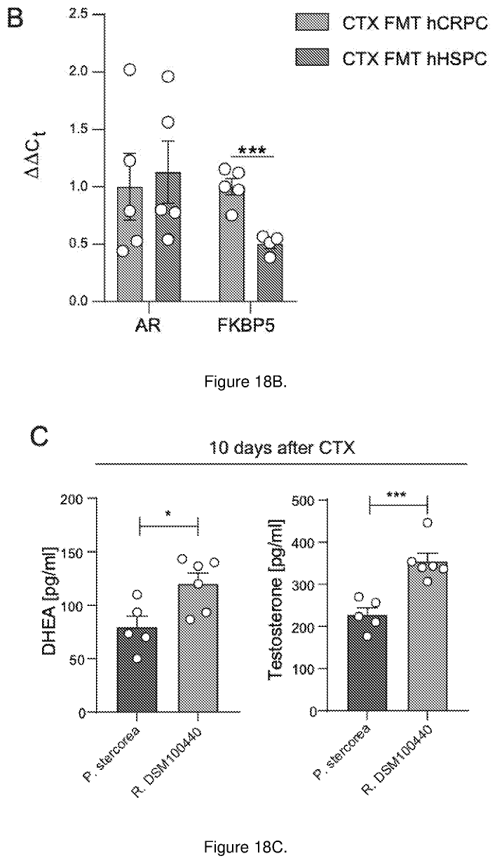

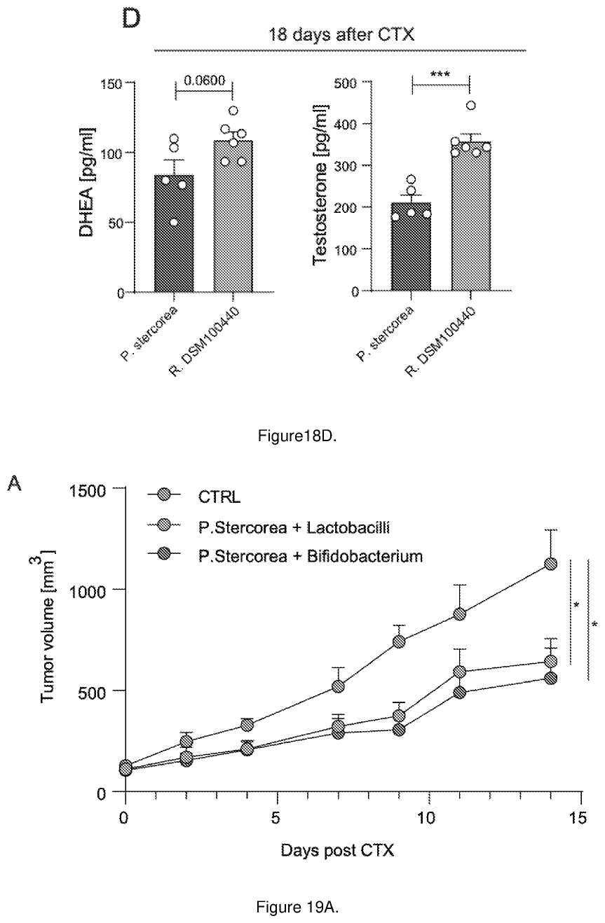

FIG. 18—Ruminococcus sp. DSM_100440 administration increases circulating androgen levels. TRAMP-C1 allograft mice were treated with ABX for 10 days before CTX, then received FMT from HSPC or CRPC patients. (A) Experimental scheme. (B) Quantitative PCR with reverse transcription (qRT-PCR) of AR and FKBP5 gene in TRAMP-C1 tumors (CTX hHSPC n=5; CTX hCRPC n=5). Targeted metabolomics analysis for DHEA and Testosterone was performed in serum of mice receiving P. stercorea (n=5) or R.DSM_100440 (n=6). (C) Serum levels of DHEA (left) and Testosterone (right) 10 days after CTX. (D) Serum levels of DHEA (left) and Testosterone (right) 18 days after CTX. Statistical analysis was performed in (B), (C), (D) with unpaired two-sided Student's t-test; *P<0.05; **P<0.01; 15 ***P<0.001; ****P<0.0001.

FIG. 19—P. stercorea, Lactobacillus and Bifobacterium consortia administration delayed tumor growth. C57BL6/N mice were challenged s.c. with 2.5×10⁶ TRAMP-C1 cells. When tumors became palpable, mice were castrated (CTX) and either left untreated (CTRL, n=5), administered with P. stercorea and Lactobacilli (n=5) or with P. stercorea and Bifidobacterium (n=5). (A) Tumor growth. C57BL6/N mice were challenged s.c. with 2.5×10⁶ TRAMP-C1 cells. When tumors became palpable, mice were castrated (CTX) and either left untreated (CTRL, n=5), administered with Lactobacilli (n=5) or Bifidobacterium (n=5). (B) Tumor growth. Statistical analysis was performed with: (A), (B) unpaired two-sided Student's t-test. +, P<0.1; *P<0.05.

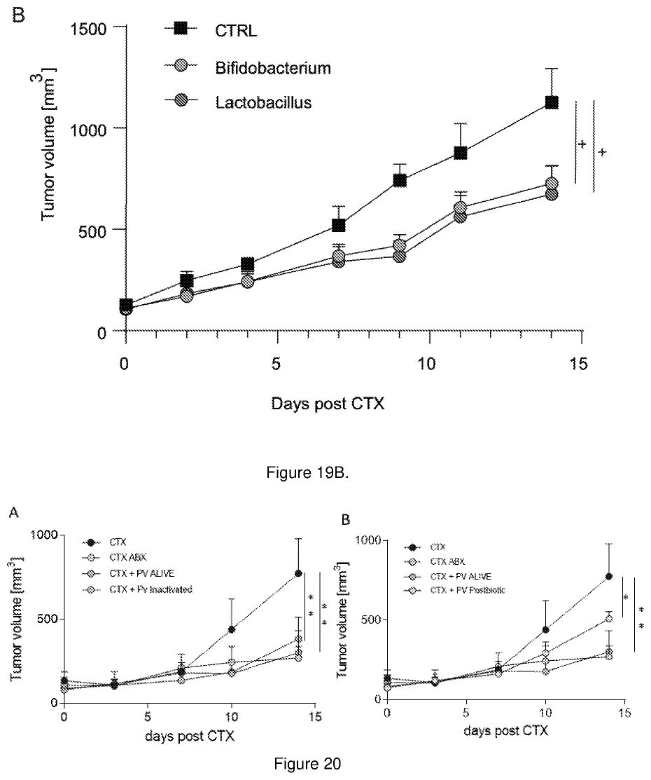

FIG. 20—Prostate cancer mice were challenged with 2.5 M Tramp-C1 cell and were castrated when the tumor was 100 mm³. Mice where either left untreated (n=5), administered with broad spectrum antibiotic cocktail (n=5), Probiotic composed of alive Prevotella stercorea (n=5), Probiotic composed of inactivated Prevotella stercorea (n=5) or postbiotic deriving from Prevotella stercorea (n=5). (A and B) Tumor volume.

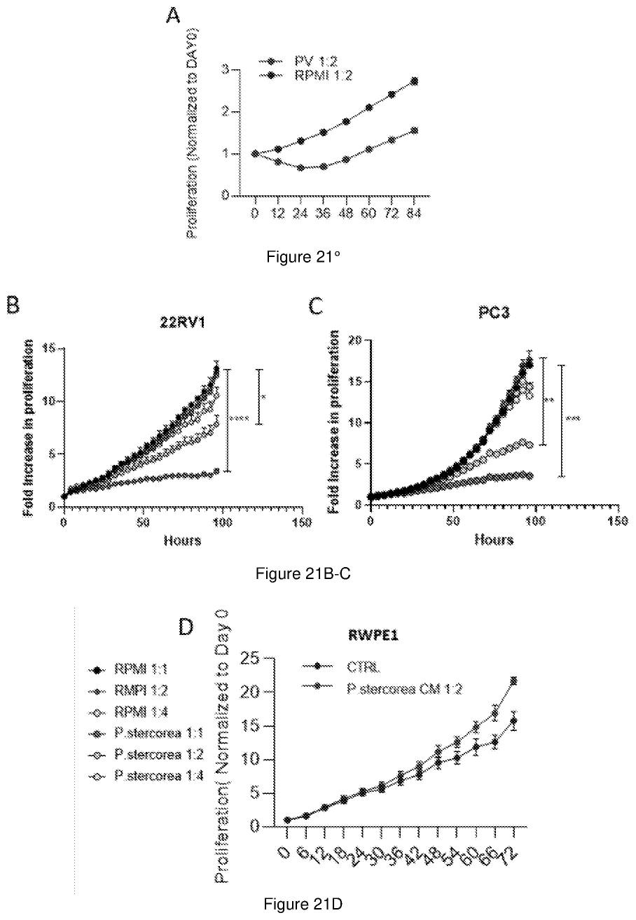

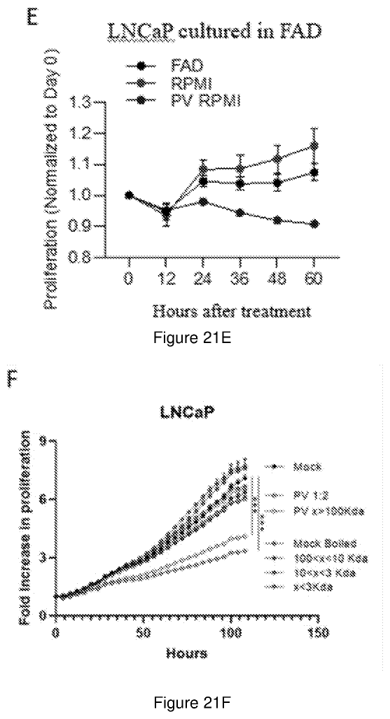

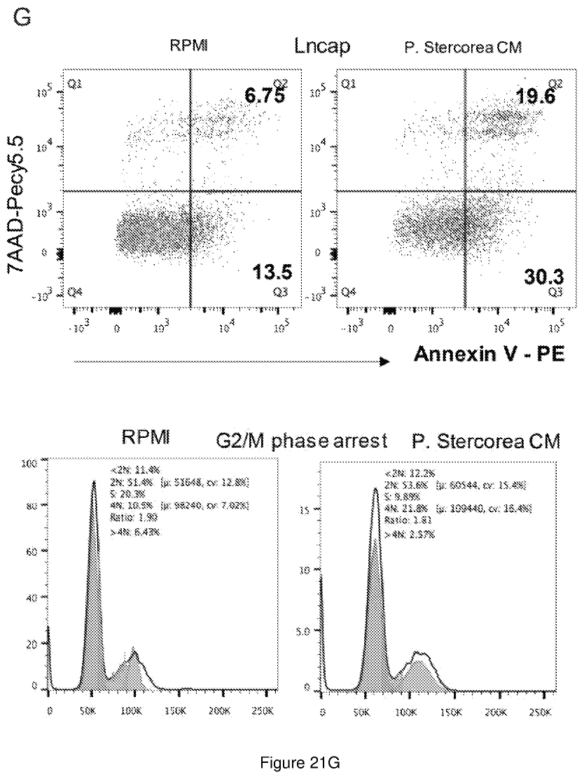

FIG. 21—Prevotella stercorea metabolic products contained in the CM inhibits Prostate Cancer cell growth in vitro. (A) Proliferation of LNCaP cells treated with vehicle (RPMI) or Prevotella CM (PV). (B) Proliferation of 22RV1 cells treated with vehicle (RPMI) or Prevotella CM (PV). (C) Proliferation of PC3 cells treated with vehicle (RPMI) or Prevotella CM (PV). (D) Proliferation of RWPE1 cells treated with vehicle or Prevotella CM (PV). (E) Proliferation of LNCaP cells kept in FAD treated with vehicle (RPMI) or Prevotella CM (PV). (F) Proliferation of LNCaP cells treated with vehicle (RPMI), Prevotella CM (PV) or different fractions obtained through concentration with filter with different cut-off sizes. Cells were exposed to PV CM or vehicle. (G) Determination of apoptotic cells through flow cytometry using AnnexinV/7AAD.

### GLOSSARY

In the context of the present description, the term “in vitro” refers to a testing method that involves experiments on biological matter (cells or tissues) outside of a living organism. In vitro experiments are historically conducted in a Petri dish and they can be conducted on a wide range of test subjects, from bacteria to cells derived from living organisms.

In the context of the present description, the term “ex vivo” means that it is done outside of a living organism. In this kind of experiments the living tissues are not created artificially but directly taken from a living organism. The experiment is then immediately conducted in a laboratory environment, with minimal alteration of the organism's natural conditions.

In the context of the present description, the term “diagnosis” refers to the process of identifying a disease, condition, or injury from its signs and symptoms. A health history, physical exam, and tests, such as blood tests, imaging tests, and biopsies, may be used to help make a diagnosis.

In the context of the present description, the term “prognosis” refers to making an educated guess about the expected outcome of any kind of health treatment, in essence making a prediction of the process an individual may have to go through in order to heal, and the extent of healing expected to take place. Prognosis is a medical term used in treatment settings based on a medical model and it is based on different factors.

In the context of the present description, the term “prostate cancer” refers to cancer that occurs in the prostate, a small walnut-shaped gland in males that produces the seminal fluid that nourishes and transports sperm. Prostate cancer is one of the most common types of cancer and while some types of prostate cancer grow slowly and may need minimal or even no treatment, other types are aggressive and can spread quickly.

In the context of the present description, the term “genus” or “genera” refers to the biological classification ranking between family and species, consisting of structurally or phylogenetically related species or a single isolated species exhibiting unusual differentiation. The genus name is the first word of a binomial scientific name (the species name is the second word).

In the context of the present description, the term “species” refers to a closely related group of organisms, which comprise similar characteristics and interbreed to produce a fertile offspring. It is considered as the fundamental unit of the classification of organisms. In order to define a particular species, the similarities in the DNA sequences, morphological, and ecological features can be considered.

In the context of the present description, the term “strain” is used to indicate a genetic variant, a subtype or a culture within a biological species. Strains are often seen as inherently artificial concepts, characterized by a specific intent for genetic isolation.

In the context of the present description, the term “inactivated” refers to a bacterial strain according to any of the embodiments disclosed herein, that has been treated using chemical or physical means so that is no longer capable of replication or reproduction in vivo or in vitro while retaining the same capability of the native strain to prevent, treat and/or inhibit the development of prostate cancer. For example, the term “inactivated” may refer, in an embodiment, to a bacterial strain that has been irradiated (ultraviolet (UV), X-ray), heated, subjected to a process such as pasteurization, tyndallization or sterilization, or chemically treated or killed so that is no longer capable of replication or reproduction in vivo or in vitro.

In the context of the present description, the term “metabolic products” denotes any substance produced by metabolism or by a metabolic process of any bacterial strain disclosed in the present disclosure, such as metabolic intermediates or metabolic end products. In particular, said metabolic products are products that retain the same capability of the strain from which they derive to prevent, treat and/or inhibit the development of prostate cancer. Metabolic products can be obtained from e.g., conditioned media, cell culture supernatants, extracts from biological samples or extracts from body fluids.

In the context of the present description, “about” refers to the experimental error that can occur during conventional measurements. More particularly, when referring to a value it indicates ±5% of the indicated value.

### DETAILED DESCRIPTION OF THE INVENTION

In the following, several embodiments of the invention will be described. It is intended that the features of the various embodiments can be combined, where compatible. In general, subsequent embodiments will be disclosed only with respect to the differences with the previously described ones.

As previously mentioned, a first object of the present invention is represented by a method for the diagnosis and/or prognosis of prostate cancer in a subject and/or for determining if a subject suffering from prostate cancer is responsive to a therapeutic treatment, comprising:
a. determining the presence of one or more bacteria selected from the group consisting of the genus Ruminococcus, the genus Streptococcus, the genus Clostridiales, the genus Prevotella, the genus Sellimonas and the genus Drancourtella in a biological sample isolated from said subject.

Ruminococcus is a genus of bacteria in the class Clostridia. They are anaerobic, Gram-positive gut microbes.

Streptococcus is a genus of gram-positive coccus or spherical bacteria that belongs to the family Streptococcaceae, within the order Lactobacillales (lactic acid bacteria), in the phylum Firmicutes. Most streptococci are oxidase-negative and catalase-negative, and many are facultative anaerobes (capable of growth both aerobically and anaerobically).

Clostridiales/clostridium is a genus of bacteria belonging to Eubacteriales order. Eubacteriales are gram-positive spherical or rod-shaped bacteria; some are motile while some are sporogenic.

Prevotella is a genus of gram-negative bacteria, non-motile, rod-shaped, singular cells that thrive in anaerobic growth conditions. They are known for being commensals, participating to the oral, vaginal and gut microbiota.

Sellimonas is a genus of gram-positive and obligately anaerobic bacteria belonging to Lachnospiranaceae order.

Dracourtella is a genus of gram-positive and obligately anaerobic bacteria, belonging to Clostidiales order. Drancourtella name was chosen in honor of the french microbiologist Michel Drancourt.

According to one embodiment of the present invention, the method is an in vitro method and, according to another embodiment, the method is an ex vivo method.

In an embodiment of the invention herein described, according to any one of the embodiments disclosed, the prostate cancer is CPRC. CRPC is a form of advanced prostate cancer where the tumor does not completely respond to treatments that lower testosterone and it shows signs of growth, like a rising PSA (prostate-specific antigen), even with low levels of testosterone.

In another embodiment of this invention, according to any one of the embodiments herein disclosed, one or more bacteria are selected from the group comprising the following species: Ruminococcus gnavus, Ruminococcus sp. DSM_100440, Ruminococcus sp. OM05_10BH, Streptococcus vestibularis, Clostridiales bacterium, Prevotella sp. BCRC_81118, Prevotella sp. Marseille_P4119, Prevotella sp. 885, Prevotella stercorea, Sellimonas intestinalis, Drancourtella massiliensis.

Ruminococcus gnavus is a strict anaerobic gram-positive coccus, which has been described as being part of the normal intestinal flora in humans.
Ruminococcus sp. DSM_100440 is a strict anaerobic gram-positive bacterium, which has been isolated from feces of a patient with inflammatory bowel disease. This bacterium is part of the normal intestine flora in humans.
Ruminococcus sp. OM05_10BH is a strict anaerobic gram-positive bacterium.
Streptococcus vestibularis is a gram-positive coccus, most streptococci are oxidase-negative and catalase-negative, and many are facultative anaerobes. S. vestibularis was first isolated from vestibular mucosa of human oral cavities.
Clostridiales bacterium is a bacterium belonging to Eubacteriales order. It is a gram-positive strictly anaerobic bacterium.
Prevotella sp. BCRC_81118, is a gram-negative bacterium, non-motile, rod-shaped, singular cells, strict anaerobic.
Prevotella sp. Marseille_P4119, is a gram-negative bacterium, non-motile, rod-shaped, singular cells, strict anaerobic.
Prevotella sp. 885, is a gram-negative bacterium, non-motile, rod-shaped, singular cells, strict anaerobic.
Sellimonas intestinalis is a gram-positive and obligately anaerobic bacteria, forming ivory yellow colonies, and was isolated from a faecal sample of a healthy Korean woman. Based on recent phylogenetic and phenotypic findings, this strain is considered to represent a novel species of a new genus belonging to the family Lachnospiraceae.
Drancourtella massiliensis, isolated from the stool of a healthy person, is a gram-positive rod-shaped bacterium, oxygen intolerant and nonmotile, with spore-forming activity.

In a further embodiment, according to any one of the embodiments herein disclosed, one or more bacteria are selected from the group comprising the following strains: Ruminococcus sp. DSM_100440, Ruminococcus sp. OM05_10BH, Streptococcus vestibularis, Clostridiales bacterium VE202_14, Prevotella sp. BCRC_81118, Prevotella sp. Marseille_P4119 and Prevotella sp. 885, Prevotella stercorea, Sellimonas intestinalis, Drancourtella massiliensis and, in another preferred embodiment, according to any one of the embodiments herein disclosed, the bacteria selected are Ruminococcus sp. DSM_100440, Ruminococcus sp. OM05_10BH, Streptococcus vestibularis, Clostridiales bacterium VE202_14, Sellimonas intestinalis, Drancourtella massiliensis

According to another embodiment of the present invention, according to any one of the embodiments herein disclosed, the biological sample is a stool sample.

In a further embodiment of the present invention, according to any one of the embodiments herein disclosed, said step a. comprises the execution of an in vitro and/or ex vivo assay selected from the group consisting of: a microbiological assay, bacterial nucleic acid sequencing assay or a combination thereof.

The expression “microbiological assay” refers to bioassays designed to analyze the compounds or substances that have impact on microorganisms. They help to estimate concentration and efficiency of antibiotics or bactericidal substances. The expression “microbiological assay” could also refer to bioassays designed to analyze any particular enzyme, protein or biological structure characteristic of a microorganism.

The expression “bacterial nucleic acid sequencing assay” refers to the process of determining the nucleic acid sequence, that is the order of nucleotides in DNA. It includes any method or technology already known in the art that is used to determine the order of the four bases: adenine, guanine, cytosine, and thymine.

Another embodiment of the invention in object, according to any one of the embodiments herein disclosed, is the method herein described wherein said step a. comprises determining the levels of said one or more bacteria by performing an amplification reaction from a nucleic acid preparation derived from said sample, using at least one pair of primers capable of amplifying at least one representative region of said genus and detecting the amplification product. In another preferred embodiment of the invention in object, according to any one of the embodiments herein disclosed, the amplification reaction is carried out by means of polymerase chain reaction.

In a further embodiment of the present invention, according to any one of the embodiments herein disclosed, the prognosis is determined in terms of at least one of: overall survival, disease- or relapse-free survival, cancer-related complications and/or rate of progression of cancer.

In another embodiment, according to any one of the embodiments herein disclosed, the presence of one or more bacteria selected from the group consisting of the genus Ruminococcus, the genus Streptococcus, the genus Clostridiales, the genus Sellimonas, the genus Drancourtella in said biological sample provides an indication of a negative prognosis and/or of the likelihood of said subject to respond to said therapeutic treatment and in another preferred embodiment, according to any one of the embodiments herein disclosed, the presence of the genus Ruminococcus, the genus Streptococcus, the genus Clostridiales, the genus Sellimonas and the genus Drancourtella in said biological sample provides an indication of a negative prognosis and/or of the likelihood of said subject to respond to said therapeutic treatment.

In a further preferred embodiment, according to any one of the embodiments herein disclosed, the presence of one or more of the strains Ruminococcus sp. DSM_100440, Ruminococcus sp. OM05_10BH, Streptococcus vestibularis, Clostridiales bacterium VE202_14, Sellimonas intestinalis and Drancourtella massiliensis in said biological sample provides an indication of a negative prognosis and/or of the likelihood of said subject to respond to said therapeutic treatment and, in an even more preferred embodiment, the concomitant presence of Ruminococcus sp. DSM_100440, Ruminococcus sp. OM05_10BH, Streptococcus vestibularis, Clostridiales bacterium VE202_14, Sellimonas intestinalis and Drancourtella massiliensis provides an indication of a poor overall survival.

In an embodiment, according to any one of the embodiments herein disclosed, the presence of one or more bacteria belonging to the genus Prevotella provides an indication of a positive prognosis. In particular, in a preferred embodiment, according to any one of the embodiments herein disclosed, the presence of one or more of the strains Prevotella sp. BCRC_81118, Prevotella sp. Marseille_P4119, Prevotella sp. 885 and Prevotella stercorea in said biological sample provides an indication of a positive prognosis and, in an even more preferred embodiment, according to any one of the embodiments herein disclosed, the concomitant presence of Prevotella sp. BCRC_81118, Prevotella sp. Marseille_P4119, Prevotella sp. 885 and Prevotella stercorea provides an indication of an improved overall survival.

In another embodiment of the present invention, according to any one of the embodiments herein disclosed, the method herein claimed, on the basis of the result of the determination in step a., further comprises selecting said subject to undergo a therapeutic treatment targeting microbiota. In particular, in a further embodiment, according to any one of the embodiments herein disclosed, said therapeutic treatment comprises fecal microbiota transplantation and/or the administration of a probiotic mixture.

The expression “fecal microbiota transplantation” refers to the administration of a solution of fecal matter from a donor into the intestinal tract of another subject in order to directly change the subject's gut microbial composition and confer a health benefit. This procedure is done via colonoscopy, enema, nasogastric (NG) tube or in capsule form. With a fecal transplant, “good” microorganisms from the donor stool are infused into the patient and, in this way, healthy bacteria begin to grow.

Probiotic mixtures are mixtures of viable microorganisms, sufficient amounts of which reach the intestine in an active state and thus exert positive health effects.

In a preferred embodiment of the invention in object, according to any one of the embodiments herein disclosed, said probiotic mixture comprises at least one bacterial strain selected from the group consisting of the genus Ruminococcus, the genus Streptococcus, the genus Clostridiales, the genus Sellimonas and the genus Drancourtella and, in an even more preferred embodiment, said probiotic mixture comprises bacterial strain selected from the group consisting of the genus Ruminococcus, the genus Streptococcus, the genus Clostridiales, the genus Sellimonas and the genus Drancourtella.

In a further embodiment, according to any one of the embodiments herein disclosed, said probiotic mixture comprises one or more of the following bacterial strains: Ruminococcus gnavus, Ruminococcus sp. DSM_100440, Ruminococcus sp. OM05_10BH, Streptococcus vestibularis, Clostridiales bacterium VE202_14, Sellimonas intestinalis and Drancourtella massiliensis and, in a further more preferred embodiment, said probiotic mixture comprises Ruminococcus gnavus, Ruminococcus sp. DSM_100440, Ruminococcus sp. OM05_10BH, Streptococcus vestibularis, Clostridiales bacterium VE202_14, Sellimonas intestinalis and Drancourtella massiliensis

In an embodiment of the invention in object, according to any one of the embodiments herein disclosed, in said probiotic mixture Ruminococcus gnavus is present at 1*10⁹-5*10¹⁰ live bacteria, Ruminococcus sp. DSM_100440 is present at 1*10⁹-5*10¹⁰ live bacteria, Ruminococcus sp. OM05_10BH is present at 1*10⁹-5*10¹⁰ live bacteria, Streptococcus vestibularis is present at 1*10⁹-5*10¹⁰ live bacteria, Clostridiales bacterium VE202_14 is present at 1*10⁹-5*10¹⁰ live bacteria, Sellimonas intestinalis is present at 1*10⁹-5*10¹⁰ live bacteria and Drancourtella massiliensis is present at 1*10⁹-5*10¹⁰ live bacteria of the total colony-forming units of the probiotic mixture. In a preferred embodiment, according to any one of the embodiments herein disclosed, in said probiotic mixture Ruminococcus gnavus is present at 8*10⁹ live bacteria, Ruminococcus sp. DSM_100440 is present at 8*10⁹ live bacteria, Ruminococcus sp. OM05_10BH is present at 5*10⁹ live bacteria, Streptococcus vestibularis is present at 5*10⁹ live bacteria, Clostridiales bacterium VE202_14 is present at 5*10⁹ live bacteria, Sellimonas intestinalis is present at 8*10⁹ live bacteria and Drancourtella massiliensis is present at 8*10⁹ live bacteria of the total colony-forming units of the probiotic mixture.

In another embodiment of the present invention, according to any one of the embodiments herein disclosed, the method herein claimed, on the basis of the result of the determination in step a., further comprises selecting said subject to undergo an anticancer treatment and, in another preferred embodiment, said anticancer treatment comprises administering to said subject a pharmaceutical composition comprising vancomycin, ampicillin, neomycin, and/or metronidazole.

Vancomycin is an antibiotic medication used to treat a number of bacterial infections. It is indicated for the treatment of serious, life-threatening infections by Gram-positive bacteria unresponsive to other antibiotics. Ampicillin is used to treat infections by many Gram-positive and Gram-negative bacteria. It was the first “broad spectrum” penicillin with activity against Gram-positive bacteria. Its spectrum of activity is enhanced by co-administration of sulbactam, a drug that inhibits beta lactamase, an enzyme produced by bacteria to inactivate ampicillin and related antibiotics. It is sometimes used in combination with other antibiotics that have different mechanisms of action, like vancomycin. Neomycin is an aminoglycoside antibiotic that displays bactericidal activity against gram-negative aerobic bacilli and some anaerobic bacilli where resistance has not yet arisen. It is generally not effective against gram-positive bacilli and anaerobic Gram-negative bacilli. Metronidazole is an antibiotic and antiprotozoal medication. It is used either alone or with other antibiotics.

In another embodiment of the present invention, according to any one of the embodiments herein disclosed, the method herein claimed, on the basis of the result of the determination in step a., further comprises selecting said subject to undergo a therapeutic treatment targeting microbiota and anticancer treatment.

A further object of the invention is a method for monitoring the response of a subject suffering from prostate cancer to a therapeutic treatment, said method comprising the steps of: a. determining and/or quantifying the levels of one or more bacteria selected from the group consisting of the genus Ruminococcus, the genus Streptococcus, the genus Clostridiales, the genus Prevotella, the genus Sellimonas and the genus Drancourtella in a biological sample isolated from the subject before said treatment;
a′. determining and/or quantifying said levels in a biological sample from the subject after said treatment; and b. based on the results of the determinations and/or quantifications performed in steps a. and
a′. determining if the subject responds to said treatment.

The expression “monitoring” refers to a periodic and systematic measurement of fixed parameters, by means of appropriate instruments, in order to monitor the situation or the trend of even complex systems, in particular if the treatment is effective and the subject is responsive to it.

According to one embodiment of the present invention, the method is an in vitro method and, according to another embodiment, the method is an ex vivo method.

In an embodiment of the present invention, according to any one of the embodiments herein disclosed, said prostate cancer is CRPC.

In another embodiment of the present invention, according to any one of the embodiments herein disclosed, said therapeutic treatment comprises administering to said subject an anticancer treatment and, in another preferred embodiment, said anticancer treatment comprises administering to said subject a pharmaceutical composition comprising vancomycin, ampicillin, neomycin, and/or metronidazole.

In a further embodiment of the present invention, according to any one of the embodiments herein disclosed, said therapeutic treatment comprises a therapeutic treatment targeting microbiota. In particular, in a further preferred embodiment, according to any one of the embodiments herein disclosed, said therapeutic treatment targeting microbiota comprises fecal microbiota transplantation and/or the administration of a probiotic mixture.

In another embodiment, according to any one of the embodiments herein disclosed, said treatment comprises administering to said subject a pharmaceutical composition comprising vancomycin, ampicillin, neomycin, and/or metronidazole, and a treatment targeting microbiota such as fecal microbiota transplantation and/or the administration of a probiotic mixture.

Another object of the present invention is a kit comprising reagents for determining the presence of one or more bacteria selected from the group consisting of the genus Ruminococcus, the genus Streptococcus, the genus Clostridiales, the genus Prevotella, the genus Sellimonas intestinalis and the genus Drancourtella in a biological sample isolated from a subject suffering from prostate cancer, preferably CRPC.

A further object of this invention is the use of said kit for the diagnosis and/or prognosis of prostate cancer in a subject, preferably of CRPC, and/or for determining if a subject suffering from prostate cancer is responsive to a therapeutic treatment, according to any one of the embodiments of the method herein disclosed.

According to one embodiment of the present invention, the diagnosis is an in vitro diagnosis and, according to another embodiment, the diagnosis is an ex vivo diagnosis.

A probiotic mixture comprising at least one bacterial strain selected from the group consisting of the genus Ruminococcus, the genus Streptococcus, the genus Clostridiales, the genus Sellimonas and/or the genus Drancourtella is another object of the present invention.

In an embodiment of the invention in object, according to any one of the embodiments herein disclosed, said probiotic mixture comprises at least one bacterial strain selected from the group consisting of the genus Ruminococcus, the genus Streptococcus, the genus Clostridiales, the genus Sellimonas and the genus Drancourtella and, in another preferred embodiment, said probiotic mixture comprises bacterial strain selected from the group consisting of the genus Ruminococcus, the genus Streptococcus, the genus Clostridiales, the genus Sellimonas and the genus Drancourtella.

Preferably, said probiotic mixture comprises at least one bacterial strain selected from the group consisting of the genus Ruminococcus, the genus Clostridiales, the genus Sellimonas and the genus Drancourtella, and optionally further comprises at least one bacterial strain selected from the genus Streptococcus.

In a further embodiment, according to any one of the embodiments herein disclosed, said probiotic mixture comprises one or more of the following bacterial strains: Ruminococcus gnavus, Ruminococcus sp. DSM_100440, Ruminococcus sp. OM05_10BH, Streptococcus vestibularis, Clostridiales bacterium VE202_14, Sellimonas intestinalis and Drancourtella massiliensis and, in a further more preferred embodiment, said probiotic mixture comprises Ruminococcus gnavus, Ruminococcus sp. DSM_100440, Ruminococcus sp. OM05_10BH, Streptococcus vestibularis, Clostridiales bacterium VE202_14, Sellimonas intestinalis and Drancourtella massiliensis

In an embodiment of the invention in object, according to any one of the embodiments herein disclosed, in said probiotic mixture Ruminococcus gnavus is present at 1*10⁹-5*10¹⁰ live bacteria, Ruminococcus sp. DSM_100440 is present at 1*10⁹-5*10¹⁰ live bacteria, Ruminococcus sp. OM05_10BH is present at 1*10⁹-5*10¹⁰ live bacteria, Streptococcus vestibularis is present at 1*10⁹-5*10¹⁰ live bacteria, Clostridiales bacterium VE202_14 is present at 1*10⁹-5*10¹⁰ live bacteria, Sellimonas intestinalis is present at 1*10⁹-5*10¹⁰ live bacteria and Drancourtella massiliensis is present at 1*10⁹-5*10¹⁰ live bacteria of the total colony-forming units of the probiotic mixture. In a preferred embodiment, according to any one of the embodiments herein disclosed, in said probiotic mixture Ruminococcus gnavus is present at 8*10⁹ live bacteria, Ruminococcus sp. DSM_100440 is present at 8*10⁹ live bacteria, Ruminococcus sp. OM05_10BH is present at 8*10⁹ live bacteria, Streptococcus vestibularis is present at 8*10⁹ live bacteria, Clostridiales bacterium VE202_14 is present at 8*10⁹ live bacteria, Sellimonas intestinalis is present at 8*10⁹ live bacteria and/or Drancourtella massiliensis is present at 8*10⁹ live bacteria of the total colony-forming units of the probiotic mixture.

In a further embodiment of the invention in object, according to any one of the embodiments herein disclosed, said probiotic mixture is for use for preventing and/or treating a disease caused by or characterized by an androgen deficiency.

The expression “androgen deficiency” refers to a pathological situation wherein the body has lower levels of male sex hormones, in particular testosterone, than is needed for good health.

In an embodiment, according to any one of the embodiments herein disclosed, said disease is accompanied by one or more of the following symptoms: depression, lethargy and fatigue, hot flushes and sweating, reduced muscle mass and strength, gynecomastia, loss of body hair, reduced bone mass.

Another object of the present invention is a probiotic mixture comprising at least one bacterial strain selected from the group consisting of the genus Prevotella, the genus Lactobacillus and the genus Bifidobacterium, for use for preventing, treating and/or inhibiting the development of prostate cancer, preferably CRPC.

Preferably, said probiotic mixture comprises at least one bacterial strain selected from the group consisting of the genus Prevotella, and optionally further comprises at least one bacterial strain selected from the genus Lactobacillus and the genus Bifidobacterium.

In one embodiment, said probiotic mixture for use comprises one or more of the following bacterial strains: Prevotella sp. BCRC_81118, Prevotella sp. Marseille_P4119, Prevotella sp. 885, Prevotella stercorea, L. casei, L. buchneri, L. acidophilus, L. paracasei, L. bulgaricus, L. rhammosum, B. bifidum, B. longum, and B. breve and, in another preferred embodiment, according to any one of the embodiments herein disclosed, said probiotic mixture for use comprises Prevotella sp. BCRC_81118, Prevotella sp. Marseille_P4119, Prevotella sp. 885, Prevotella stercorea, L. casei, L. buchneri, L. acidophilus, L. paracasei, L. bulgaricus, L. rhammosum, B. bifidum, B. longum, and B. breve.

According to another embodiment, said probiotic mixture for use comprises only bacterial strain selected from the group consisting of the genus Prevotella and, according to a preferred embodiment, according to any one of the embodiments herein disclosed, said probiotic mixture for use comprises one or more of the following bacterial strains: Prevotella sp. BCRC_81118, Prevotella sp. Marseille_P4119, Prevotella sp. 885 and/or Prevotella stercorea.

In a further embodiment of the invention in object, according to any one of the embodiments herein disclosed, in said probiotic mixture for use Prevotella sp. BCRC_81118 is present at 1*10⁹-5*10¹⁰ live bacteria, Prevotella sp. Marseille_P4119 is present at 1*10⁹-5*10¹⁰ live bacteria, Prevotella sp. 885 is present at 1*10⁹-5*10¹⁰ live bacteria, and Prevotella stercorea is present at 1*10⁹-5*10¹⁰ live bacteria of the total colony-forming units of the probiotic mixture. In a further preferred embodiment, according to any one of the embodiments herein disclosed, in said probiotic mixture for use Prevotella sp. BCRC_81118 is present at 1*10¹⁰ live bacteria, Prevotella sp. Marseille_P4119 is present at 1*10¹⁰ live bacteria, Prevotella sp. 885 is present at 1*10¹⁰ live bacteria, and Prevotella stercorea is present at 1*10¹⁰ live bacteria of the total colony-forming units of the probiotic mixture.

In an embodiment of the present invention, according to any one of the embodiments herein disclosed, said probiotic mixture for use is in association with one or more of the following antibiotics: vancomycin, ampicillin, neomycin, and/or metronidazole and, according to a preferred embodiment, said probiotic mixture is in association with vancomycin, ampicillin, neomycin and/or metronidazole.

A further object of this invention is a postbiotic mixture comprising at least one inactivated bacterial strain selected from the genus Prevotella or a metabolic product thereof. According to a preferred embodiment, a postbiotic mixture according to any of the embodiments disclosed herein is for use for preventing, treating and/or inhibiting the development of prostate cancer, in particular castration-resistant prostate cancer.

According to a preferred aspect of the invention, the at least one inactivated bacterial strain that is present in said postbiotic mixture is Prevotella stercorea. Merely by way of example, said strain selected from the genus Prevotella can be inactivated by subjecting it to pasteurization at 70° C., for 30 minutes, or else to tyndallization or sterilization.

A further object of this invention is a composition comprising said probiotic mixture as described by any one of the embodiments herein and at least one suitable excipient and/or additive.

In one embodiment, said composition is for oral use and, in another embodiment, according to any one of the embodiments herein disclosed, said composition is in a solid semisolid, liquid or semiliquid form.

In a further embodiment, according to any one of the embodiments herein disclosed, said composition is in the form of a tablet, hard or soft capsule, pill, gelatin, lozenge, powder, granules, sachet, film, drops, suspension, emulsion, solution, syrup, or elixir and, in a further preferred embodiment, said composition is in the form of a capsule or tablet.

In another preferred embodiment, according to any one of the embodiments herein disclosed, said capsule or tablet are gastro-resistant capsule or tablet, and in a further preferred embodiment, according to any one of the embodiments herein disclosed, said capsule or tablet are delayed-release capsule or tablet.

Object of the present invention is also a pharmaceutical composition comprising one or more antibiotics suitable to kill one or more bacteria selected from the group consisting of the genus Ruminococcus, the genus Streptococcus, the genus Clostridiales, the genus Prevotella, the genus Sellimonas and the genus Drancourtella, in particular Ruminococcus sp. DSM_100440, Ruminococcus sp. OM05_10BH, Streptococcus vestibularis, Clostridiales bacterium VE202_14, Sellimonas intestinalis and Drancourtella massiliensis for use for preventing, treating and/or inhibiting the development of prostate cancer, preferably CRPC.

In one embodiment the pharmaceutical composition comprising vancomycin, ampicillin, neomycin, and/or metronidazole for use for preventing, treating and/or inhibiting the development of prostate cancer, preferably CRPC.

In one embodiment, said pharmaceutical composition is for oral use and, in another embodiment, according to any one of the embodiments herein disclosed, said pharmaceutical composition is in a solid semisolid, liquid or semiliquid form.

In a further embodiment, according to any one of the embodiments herein disclosed, said pharmaceutical composition is in the form of a tablet, hard or soft capsule, pill, gelatin, lozenge, powder, granules, sachet, film, drops, suspension, emulsion, solution, syrup, or elixir and, in a further preferred embodiment, said pharmaceutical composition is in the form of a capsule or tablet.

In another preferred embodiment, according to any one of the embodiments herein disclosed, said capsule or tablet are gastro-resistant capsule or tablet, and in a further preferred embodiment, according to any one of the embodiments herein disclosed, said capsule or tablet are delayed-release capsule or tablet.

Object of the present invention is also a method of treatment of CRPC based on the result of the determination in step a. and a′. of the in vitro and/or ex vivo methods herein disclosed, according to any one of the embodiments herein described.

In an embodiment, said method of treatment targets microbiota. In particular, in another embodiment, according to any one of the embodiments herein disclosed, said method of treatment comprises fecal microbiota transplantation and/or the administration of a probiotic mixture as herein disclosed.

In an embodiment of the present invention, according to any one of the embodiments herein disclosed, said probiotic mixture is in association with one or more of the following antibiotics: vancomycin, ampicillin, neomycin, and/or metronidazole and, according to a preferred embodiment, said probiotic mixture is in association with vancomycin, ampicillin, neomycin and metronidazole.

A further object of the invention is the use in association of the probiotic or a postbiotic mixture according to any embodiments herein disclosed with a drug for the androgen deprivation therapy for preventing, treating and/or inhibiting the development of prostate cancer, in particular castration-resistant prostate cancer (CRPC). In particular, said drug for the androgen deprivation therapy is selected from a LHRH analog or antagonist.

A further object of the invention is the use in association of the probiotic or a postbiotic mixture according to any embodiments herein disclosed a drug for the hormonal therapy for use for preventing, treating and/or inhibiting the development of prostate cancer, in particular castration-resistant prostate cancer (CRPC). In particular, said drug for the hormonal therapy is selected from Bicalutamide, Enzalutamide, Abiraterone, Apalutamide In any part of the present description and claims the terms comprising/comprises/comprise can be substituted by the term “consisting of/consists of/consist of”.

Examples are reported below which have the purpose of better illustrating the methodologies disclosed in the present description, such examples are in no way to be considered as a limitation of the previous description and the subsequent claims.

### EXAMPLES

### Example 1—Depletion of the Intestinal Microbiota in Castrated but not in Sham-Operated Mice Affects CRPC Growth

To address the impact of the intestinal microbiota on CRPC progression, two mouse models have been empolyed: the TRAMP-C1 allograft and the Pten^{pc-/-} prostate conditional mouse models. In both models, surgical castration (CTX) is followed by a castration-sensitive phase (CS; in Pten^{pc-/-} mice 4 weeks after CTX i.e. 12 weeks of age; in TRAMP-C1 allograft mice 6 days after CTX) in which the tumor size shrinks because of androgen ablation, and a subsequent castration-resistant phase (CR; in Pten^{pc-/-} mice 12 weeks after CTX i.e. 20 weeks of age; in TRAMP-C1 allograft mice 10 days after CTX) in which the tumor becomes resistant to androgen ablation and starts to grow again. To deplete the intestinal microbiota, the inventors treated these tumor-bearing mice with a cocktail of wide-spectrum antibiotics (ABX) (F. Fransen et al., BALB/c and C57BL/6 Mice Differ in Polyreactive IgA Abundance, which Impacts the Generation of Antigen-Specific IgA and Microbiota Diversity. Immunity 43, 527-540 (2015)) that reduced the fecal CFU counts of about 10 Log (FIG. 5, A and B). Microbiota ablation resulted in delayed tumor growth and improved survival in the TRAMP-C1 CTX context, but not in sham-operated animals (FIG. 1, A and B), with no impact on animal weight (FIG. 5C). Microbiota ablation significantly reduced Ki67 positive prostate cancer cells in tumors of castration-resistant mice without altering the percentage of apoptotic cells as detected by cleaved caspase 3 (cC3) positivity (FIG. 1C and FIG. 5, D and E). Also in the Pten^{pc-/-} CTX model, microbiota ablation led to a reduction in prostate tumor volume as detected by magnetic resonance imaging (MRI) measurement of tumor volume over a time course, with no effect on sham-operated animals (FIG. 1, D and E).

Histopathological evaluation of the anterior prostate (AP) lobe showed a clear reduction in tumor aggressiveness (percentage of glands with invasive area) in mice treated with ABX (FIG. 5, F and G). Loss of intestinal microbiota led to a reduction of Ki67 positive cells in castration resistant-mice but did not impact cell proliferation in sham-operated animals (FIG. 5H). Of note, ABX treatment of prostate epithelial tumor cells cultured in 2D did not impair tumor cell growth (FIG. 5I). These data were validated in vivo in two additional CS mouse models of human prostate cancer: the LNCaP xenograft model and the patient-derived xenograft (PDX) model LuCaP35. In both cases, ABX resulted in delayed onset of CRPC that improved survival (FIG. 1 F-I). Similar results were acquired in CTX mice treated with enzalutamide, a second-generation androgen receptor antagonist (FIG. 6, A and B). Overall, these data demonstrated that elimination of the intestinal microbiota impacts prostate tumorigenesis in different mouse models, selectively in castrated mice.

The inventors next hypothesized that CTX altered mouse intestinal microbiota. To address this point, they performed 16S rDNA sequencing on fecal DNA from sham-operated and castrated Pten^{pc-/-} mice. The inventors identified a compositional difference in the two cohorts (FIG. 7A), with enrichment of specific microbiota species in both CR and CS mice (FIG. 1J and FIG. 7B). Specifically, two species, namely Ruminococcus gnavus and Bacteroides acidifaciens, were particularly enriched in the fecal microbiota of CR Pten^{pc-/-} mice (FIG. 1K). Ruminococcus gnavus was also found enriched in fecal samples of castrated LNCaP mice, including those treated with enzalutamide (FIG. 7, C-E). Intriguingly, bipolar androgen therapy in the TRAMP-C1 mouse model promoted tumor inhibition and decreased the abundance of Ruminococcaceae family, thereby demonstrating that changes in circulating host androgens impact gut microbiota composition (FIG. 7, F-H).

Given the reported dependence of the immune response on the composition of the gut microbiota, the inventors have analyzed the systemic and tumor-infiltrating immune cell populations in castrated mice but found that ABX treatment induced only minimal variations in circulating level of cytokines (FIG. 8A), percentage of tumor-infiltrating immune subsets (FIG. 8B) and percentages of immune cell subsets in other organs (FIG. 8 C-G). In confirmation, ABX treatment was also effective in TRAMP-C1 allograft mice treated with an anti-Ly6G depleting antibody targeting tumor-infiltrating myeloid cells (FIG. 9A) and in NOD-SCID mice lacking T, B and NK cells (FIG. 9B). Overall, these data demonstrated that the intestinal microbiota is altered in CRPC and sustain tumor growth without regulating systemic and local immune responses.

### Example 2—Fecal Microbiota Transplantation from CRPC Mice Supports Tumor Growth in Castrated Recipient Mice

To better dissect the functional role of the intestinal microbiota in sustaining CRPC growth, fecal microbiota transplantation (FMT) experiments were performed in TRAMP-C1 allograft recipients with feces from mice that had become CR from previous treatment (CR FMT) or from wild type mice (HD FMT) and the evolution of the tumor volume upon castration was followed. To avoid competition with the endogenous microbiota, recipient mice were pre-treated with ABX for 7 days before CTX and FMT (FIG. 10A). FMT successfully engrafted in the hosts (FIG. 10, B and C). While CR FMT resulted in the rapid emergence of CRPC, HD FMT controlled tumor growth to levels comparable to ABX treatment (FIG. 2A).

Notably, CR FMT also significantly impacted survival (FIG. 2B). CR FMT induced an increase in tumor cell proliferation as measured by Ki67 staining, whereas HD FMT was associated with a decreased Ki67 staining (FIG. 2C and FIG. 10D). Both treatments did not alter the percentage of apoptotic (cC3 positive) tumor cells (FIG. 10E). These data were also validated in CTX Pten^{pc-/-} mice treated with either CR or HD FMT (FIG. 2, D-G and FIG. 10F). Histopathological evaluation of AP lobes showed a significant reduction in the frequency of adenocarcinoma and invasive cancer in HD FMT treated CTX Pten^{pc-/-} mice as compared to those receiving CR FMT (FIG. 2H). In the FMT setting, we also did not find major differences in the relative abundance of tumor-infiltrating immune cell populations (FIG. 10 G-J).

Since the metagenomic analysis of the CR microbiota had revealed specific enrichment in R. gnavus and B. acidifaciens, we tested the functional impact of these two species in promoting CRPC growth by performing colonization experiments. TRAMP-C1 allograft mice were treated with ABX for 7 days prior to CTX to eliminate the competing endogenous microbiota. After CTX, mice were either left untreated or administered every other day orally with R. gnavus or B. acidifaciens. Administration of R. gnavus increased tumor growth compared to untreated animals (FIG. 2I), while B. acidifaciens alone did not support tumor growth in this context as it was unable to colonize recipient mice (data not shown). Overall, these data show that the murine CR microbiota and R. gnavus are able to sustain tumor growth, while HD FMT delays the onset of CRPC.

### Example 3—the CRPC Microbiota is Enriched in Bacterial Species that Produce Androgens and Impact on Tumor Cell Growth

As the intestinal microbiota is known to impact the host's metabolome, the inventors performed untargeted metabolomic analyses of sera from Pten^{pc-/-} CTX mice treated or not with ABX. This revealed different metabolomic profiles in the two cohorts (FIG. 11A). Interestingly, the inventors detected a significant reduction in circulating dehydroepiandrosterone (DHEA) and testosterone in microbiota-depleted animals, even if the abundance of the upstream metabolite pregnenolone was not altered (FIG. 11B). In line with these findings, the expression levels of AR target genes were significantly reduced in animals devoid of intestinal microbiota (FIG. 11C). Targeted metabolomic analysis in Pten^{pc-/-} mice confirmed that both DHEA and testosterone were decreased in CTX mice treated with ABX, whereas in intact mice ABX treatment did not alter androgen levels (FIG. 3, A and B). Moreover, CR FMT and R. gnavus administration in TRAMP-C1 mice resulted in increased circulating DHEA and testosterone levels when compared to HD FMT treatment (FIG. 3, C and D).

The inventors next assessed whether the bacteria found enriched in the gut of CR mice were capable of synthesizing androgenic steroids. A bacterial strain isolated from the human microbiota, Clostridium scindens, converted glucocorticoids into androgens. The inventors, therefore, hypothesized that R. gnavus and B. acidifaciens could have a similar metabolic capability. To prove this, they cultured these two strains in the presence of a panel of metabolites of the androgen biosynthesis pathway: cholesterol (the progenitor metabolite of the androgen synthesis pathway), pregnenolone, hydroxypregnenolone, cortisol, and aldosterone (FIG. 11D). They analyzed the ability of bacterial species to convert in vitro these compounds into other intermediates of the pathway by using LC-MS/MS (FIG. 11E). As negative controls, the inventors tested seven bacterial strains indigenous to the gut microbiota (E. cloacae, K. pneumoniae 27, P. mirabilis, S. marcescens, S. haemoliticus, E. coli). Interestingly, it was found that only R. gnavus and B. acidifaciens converted pregnenolone and hydroxypregnenolone, but not other metabolites, into downstream metabolites of the pathway, including DHEA and testosterone (FIG. 3, E and F and data not shown). The inventors then tested the ability of conditioned media (C.M.) of R. gnavus incubated with pregnenolone to activate the AR signaling on TRAMP-C1 cells cultured in full-androgen deprivation (FAD) conditions (FIG. 3G).

The C.M. of R. gnavus incubated with culture broth alone was used as a control. Expression of AR target genes highlighted the ability of R. gnavus C.M., cultured in the presence of pregnenolone, to stimulate the transcription of AR target genes as compared to control C.M. (FIG. 3H). Given that R. gnavus and B. acidifaciens were capable of converting pregnenolone to DHEA and testosterone in vitro, and that these species increased in the gut microbiota of CR mice, the inventors hypothesized that, in CTX mice, circulating pregnenolone reaches the gut through the enterohepatic circulation, is metabolized to DHEA and testosterone by the gut microbiota, which are then re-adsorbed into the bloodstream, through which they can reach the prostate, thereby contributing to tumor growth (FIG. 3I). Importantly, in CTX mice and CTX patients, cholesterol can be metabolized into pregnenolone by the adrenal glands (E. A. Mostaghel et al., Contribution of Adrenal Glands to Intratumor Androgens and Growth of Castration-Resistant Prostate Cancer. Clin Cancer Res 25, 426-439 (2019)). The inventors, therefore, proved this hypothesis in vivo in CTX Pten^{pc-/-} mice by injecting deuterated (^{D}) pregnenolone intravenously into mice treated or untreated with ABX to deplete the intestinal microbiota (FIG. 12A). The abundance of ^{D}pregnenolone that reached the intestine (hence the microbiota) after the injection was comparable in both groups (FIG. 12B). The conversion of this substrate into the downstream deuterated metabolites was monitored in the blood over a time-course experiment and detected with LC-MS/MS. Strikingly, the inventors observed that in CTX mice ablation of the microbiota resulted in a significant reduction of circulating ^{D}DHEA and ^{D}testosterone, particularly at 2 and 6 hours post-injection, compared to control mice both in CR (FIG. 3, J and K) and in CS phases (FIG. 12, C and D). To corroborate these data, they assessed whether microbiota depletion by ABX was still effective in a prostate tumor model lacking the AR (PC3), and in a model insensitive to castration (LUCaP145.2) and found that in these contexts, ABX treatment was ineffective (FIG. 13, A-D). These data were further validated in LUCaP145.2 mice castrated and treated with Enzalutamide. In these mice androgen-deprivation induced a significant expansion of fecal R. gnavus and B. acidifaciens (FIG. 13, E and F). However, the expansion of these bacteria did not impact on the growth of these androgen-insensitive tumors. Altogether, these data showed that species enriched in the murine CR microbiota can participate in androgen metabolism in vitro and in vivo, and that this metabolic activity sustains androgen-dependent prostate tumor growth.

### Example 4—Metagenomic Analysis of Human CRPC Fecal Samples Identifies Bacterial Species that Produce Androgens and Promote Castration Resistance in Gut-Humanized Mice

To validate the translational potential of the previous findings, the inventors analyzed the gut microbiota of two cohorts of patients with HSPC (n=19) and metastatic CRPC (n=55) (FIG. 14, A and B, Table 1, Table 2).

TABLE 1
Patients' characteristics

| CLINICAL DATA (at time of swab) | HSPC (n = 19) | CRPC (n = 55) |
|---|---|---|
| Age (years; mean ± SD) | 69.84 ± 11.45 | 69.03 ± 11.51 |
| PSA (μg/L; mean ± SD) | 14.03 ± 28.51 | 176.89 ± 399.24 |
| NLR (mean ± SD) | 2.7 ± 1.05 | 3.78 ± 2.66 |
| Gleason at diagnosis (mean ± SD) | 7.43 ± 2.12 | 8.06 ± 1.63 |
| Overall survival (months; mean ± | 9.7 ± 3.12 | 8.8 ± 2.98 |
| SD) | | |
| Enzalutamide/Abiraterone | 4/19 | 20/55 |
| Docetaxel/Cabazitaxel | 0/19 | 5/55 |
| Metastasis | 12/19 | 51/55 |

TABLE 2
Characteristics of the patients involved in the study.

| | | | | | | | OS | | |
|---|---|---|---|---|---|---|---|---|---|
| | | | | | | GLEA- | (from | | |
| Patient | Dis- | ORI- | | PSA | | SON | swab) | Sta- | |
| ID | ease | GIN | AGE | (μg/L) | NLR | score | MONTHS | tus | ENZALUTAMIDE |
| 1043171- | HSPC | CH | 52 | NA | 2.90 | 7 | 11 | live | 0 |
| CS_R1 | | | | | | | | | |
| 1111305- | HSPC | CH | 72 | NA | 2.67 | 8 | 10 | live | 0 |
| CS_R1 | | | | | | | | | |
| 1225699- | HSPC | CH | 83 | 0.27 | 1.18 | 8 | 11 | live | 0 |
| CS_R1 | | | | | | | | | |
| 1267083- | HSPC | CH | 64 | 0.01 | 2.16 | NA | 13 | live | 0 |
| CS_R1 | | | | | | | | | |
| 200349549- | HSPC | CH | 63 | NA | 2.56 | 7 | 10 | live | 0 |
| CS_R1 | | | | | | | | | |
| 200435639- | HSPC | CH | 54 | 34.80 | 3.18 | 9 | 12 | live | 0 |
| CS_R1 | | | | | | | | | |
| 200613349- | HSPC | CH | 52 | 0.01 | 1.49 | 9 | 13 | live | 0 |
| CS_R1 | | | | | | | | | |
| 200649858- | HSPC | CH | 62 | 101 | 2.68 | 9 | 6 | dead | 0 |
| CS_R1 | | | | | | | | | |
| 200651216- | HSPC | CH | 88 | 52.10 | 3.31 | 9 | 8 | live | 0 |
| CS_R1 | | | | | | | | | |
| 2095639- | HSPC | CH | 86 | 0.25 | 2.34 | | 13 | live | 0 |
| CS_R1 | | | | | | | | | |
| 2106451- | HSPC | CH | 55 | 0.20 | 3.60 | 8 | 10 | live | 0 |
| CS_R1 | | | | | | | | | |
| 2121694- | HSPC | CH | 78 | 1.45 | 6.08 | 9 | 10 | live | 0 |
| CS_R1 | | | | | | | | | |
| 2148110- | HSPC | CH | 79 | 1.17 | 3.38 | NA | 11 | live | 0 |
| CS_R1 | | | | | | | | | |
| 2185127- | HSPC | CH | 70 | 2.69 | 2.60 | 7 | 12 | live | 0 |
| CS_R1 | | | | | | | | | |
| 2191913- | HSPC | CH | 76 | NA | 2.67 | 8 | 9 | live | 0 |
| CS_R1 | | | | | | | | | |
| 4013825- | HSPC | CH | 60 | NA | 2.30 | 7 | 9 | live | 0 |
| CS_R1 | | | | | | | | | |
| 6043029- | HSPC | CH | 78 | 1.08 | 2.61 | 8 | 11 | live | 0 |
| CS_R1 | | | | | | | | | |
| V6014- | HSPC | UK | 76 | 1.20 | 2.71 | 0 | 0 | live | 0 |
| CS_R1 | | | | | | | | | |
| V6015- | HSPC | UK | 79 | 0.23 | 0.88 | 6 | 5 | live | 0 |
| CS_R1 | | | | | | | | | |
| 1053432- | CRPC | CH | 67 | 16.8 | NA | 9 | 11 | live | 0 |
| CR_R1 | | | | | | | | | |
| 1059770- | CRPC | CH | 77 | NA | 6.01961 | 7 | 4 | dead | 0 |
| CR_R1 | | | | | | | | | |
| 1150864- | CRPC | CH | 85 | 143 | 6.30488 | NA | 11 | live | 1 |
| CR_R1 | | | | | | | | | |
| 2002623- | CRPC | CH | 71 | 22.6 | 5.54706 | 8 | 13 | live | 0 |
| CR_R1 | | | | | | | | | |
| 2003068- | CRPC | CH | 88 | 29.7 | 6.95536 | | 5 | dead | 1 |
| CR_R1 | | | | | | | | | |
| 200439709- | CRPC | CH | 71 | 242 | 7.72093 | 8 | 13 | live | 0 |
| CR_R1 | | | | | | | | | |
| 200528369- | CRPC | CH | 71 | 1064 | 2.84962 | 8 | 0 | dead | 0 |
| CR_R1 | | | | | | | | | |
| 2028781- | CRPC | CH | 80 | 12.9 | 4.125 | 8 | 11 | dead | 1 |
| CR_R1 | | | | | | | | | |
| 2074289- | CRPC | CH | 68 | 11.7 | 1.58333 | 9 | 13 | live | 1 |
| CR_R1 | | | | | | | | | |
| 2183640- | CRPC | CH | 70 | 40.4 | 0.97561 | | 12 | live | 1 |
| CR_R1 | | | | | | | | | |
| 3020211- | CRPC | CH | 69 | 6.12 | 2.93814 | 8 | 11 | live | 1 |
| CR_R1 | | | | | | | | | |
| 7012328- | CRPC | CH | 70 | 496 | 2.70443 | 9 | 13 | live | 0 |
| CR_R1 | | | | | | | | | |
| V5039- | CRPC | UK | 77 | 2614 | 2.8972 | 7 | 10 | dead | 0 |
| CR_R1 | | | | | | | | | |
| V5225- | CRPC | UK | 73 | 1.2 | 1.68571 | 6 | 11 | live | 1 |
| CR_R1 | | | | | | | | | |
| V5422- | CRPC | UK | 75 | 882 | 3.15741 | 8 | 6 | dead | 0 |
| CR_R1 | | | | | | | | | |
| V5446- | CRPC | UK | 69 | 0.004 | 5.08649 | 9 | 10 | live | 0 |
| CR_R1 | | | | | | | | | |
| V5464- | CRPC | UK | 64 | 0.004 | 2.1875 | 7 | 10 | live | 0 |
| CR_R1 | | | | | | | | | |
| V5523- | CRPC | UK | 75 | 74 | 5.81905 | NA | 11 | live | 0 |
| CR_R1 | | | | | | | | | |
| V5667- | CRPC | UK | 70 | 7.7 | 8.69091 | NA | 4 | dead | 0 |
| CR_R1 | | | | | | | | | |
| V5707- | CRPC | UK | 67 | 42 | 2.76923 | 9 | 11 | live | 0 |
| CR_R1 | | | | | | | | | |
| V5718- | CRPC | UK | 66 | 1.6 | 1.72881 | 9 | 10 | live | 0 |
| CR_R1 | | | | | | | | | |
| V5743- | CRPC | UK | 76 | 30 | 13.5 | 10 | 5 | dead | 0 |
| CR_R1 | | | | | | | | | |
| V5765- | CRPC | UK | 68 | 336 | 3.12255 | NA | 11 | live | 1 |
| CR_R1 | | | | | | | | | |
| V5777- | CRPC | UK | 66 | 27 | 1.59394 | 7 | 5 | live | 0 |
| CR_R1 | | | | | | | | | |
| V5781- | CRPC | UK | 68 | 194 | 3.5678 | 9 | 9 | dead | 0 |
| CR_R1 | | | | | | | | | |
| V5795- | CRPC | UK | 73 | 68 | 1.40969 | 8 | 6 | dead | 0 |
| CR_R1 | | | | | | | | | |
| V5796- | CRPC | UK | 61 | 16 | 1.90698 | 9 | 9 | live | 0 |
| CR_R1 | | | | | | | | | |
| V5810- | CRPC | UK | 66 | 5.5 | 1.22222 | 9 | 11 | live | 1 |
| CR_R1 | | | | | | | | | |
| V5812- | CRPC | UK | 69 | 99 | 1.58152 | 9 | 4 | dead | 0 |
| CR_R1 | | | | | | | | | |
| V5821- | CRPC | UK | 58 | 0.22 | 2.33333 | 9 | 10 | live | 0 |
| CR_R1 | | | | | | | | | |
| V5831- | CRPC | UK | 0 | 0 | 2.78947 | 0 | | live | 0 |
| CR_R1 | | | | | | | | | |
| V5856- | CRPC | UK | 81 | 52 | 6.8 | 7 | 9 | dead | 0 |
| CR_R1 | | | | | | | | | |
| V5860- | CRPC | UK | 64 | 245 | NA | 9 | 10 | dead | 0 |
| CR_R1 | | | | | | | | | |
| V5867- | CRPC | UK | 79 | 219 | 4.2027 | 6 | 11 | live | 0 |
| CR_R1 | | | | | | | | | |
| V5875- | CRPC | UK | 68 | 0.004 | 2.27586 | 7 | 9 | live | 0 |
| CR_R1 | | | | | | | | | |
| V5878- | CRPC | UK | 68 | 148 | 2.03646 | 8 | 10 | live | 0 |
| CR_R1 | | | | | | | | | |
| V5882- | CRPC | UK | 60 | 109 | 3.29299 | 9 | 11 | live | 1 |
| CR_R1 | | | | | | | | | |
| V5890- | CRPC | UK | 64 | 4.9 | 2.4537 | 7 | 10 | live | 0 |
| CR_R1 | | | | | | | | | |
| V5901- | CRPC | UK | 85 | 1.9 | 2.19549 | 5 | 12 | live | 1 |
| CR_R1 | | | | | | | | | |
| V5928- | CRPC | UK | 69 | 310 | 3.09091 | 9 | 9 | live | 0 |
| CR_R1 | | | | | | | | | |
| V5929- | CRPC | UK | 61 | 667 | 1.47059 | 9 | 5 | live | 1 |
| CR_R1 | | | | | | | | | |
| V5935- | CRPC | UK | 77 | 135 | 3.91262 | 7 | 7 | dead | 0 |
| CR_R1 | | | | | | | | | |
| V5954- | CRPC | UK | 80 | 47 | 3.75484 | 9 | 12 | live | 0 |
| CR_R1 | | | | | | | | | |
| V5968- | CRPC | UK | 66 | 509 | 11.9643 | 9 | 3 | dead | 0 |
| CR_R1 | | | | | | | | | |
| V5969- | CRPC | UK | 66 | 10 | 2.06034 | 10 | 8 | live | 1 |
| CR_R1 | | | | | | | | | |
| V5970- | CRPC | UK | 65 | 84 | 1.89873 | 7 | 10 | live | 0 |
| CR_R1 | | | | | | | | | |
| V5971- | CRPC | UK | 75 | 0.04 | 4.75 | 9 | 3 | dead | 0 |
| CR_R1 | | | | | | | | | |
| V5972- | CRPC | UK | 65 | 193 | 8.94318 | 9 | 10 | dead | 0 |
| CR_R1 | | | | | | | | | |
| V5973- | CRPC | UK | 73 | 3.1 | 1.66832 | 10 | 11 | live | 0 |
| CR_R1 | | | | | | | | | |
| V5979- | CRPC | UK | 68 | 95 | 3.87413 | 9 | 7 | live | 1 |
| CR_R1 | | | | | | | | | |
| V5980- | CRPC | UK | 57 | 2 | 1.89224 | 9 | 8 | live | 0 |
| CR_R1 | | | | | | | | | |
| V5985- | CRPC | UK | 62 | 174 | 2.55556 | 9 | 7 | live | 0 |
| CR_R1 | | | | | | | | | |
| V5986- | CRPC | UK | 67 | 4.2 | 2.52941 | 9 | 6 | live | 0 |
| CR_R1 | | | | | | | | | |
| V5991- | CRPC | UK | 76 | 49 | 6.72727 | | 9 | dead | 0 |
| CR_R1 | | | | | | | | | |
| V6002- | CRPC | UK | 73 | 6.8 | 1.25806 | 6 | 9 | live | 0 |
| CR_R1 | | | | | | | | | |

| | | | | Met | | |
|---|---|---|---|---|---|---|
| | | | | At | Met | TESTOS- |
| Patient | | | | DIAG- | at | TERONE |
| ID | ABIRATERONE | DOCETAXEL | CABXITAXEL | NOSIS | SWAB | group |
| 1043171- | 0 | 0 | 0 | 0 | 0 | NA |
| CS_R1 | | | | | | |
| 1111305- | 0 | 0 | 0 | 0 | 0 | NA |
| CS_R1 | | | | | | |
| 1225699- | 1 | 0 | 0 | 1 | 1 | NA |
| CS_R1 | | | | | | |
| 1267083- | 0 | 0 | 0 | 1 | 1 | NA |
| CS_R1 | | | | | | |
| 200349549- | 0 | 0 | 0 | 0 | 0 | NA |
| CS_R1 | | | | | | |
| 200435639- | 1 | 0 | 0 | 1 | 1 | NA |
| CS_R1 | | | | | | |
| 200613349- | 0 | 0 | 0 | 0 | 0 | NA |
| CS_R1 | | | | | | |
| 200649858- | 1 | 0 | 0 | 1 | 1 | NA |
| CS_R1 | | | | | | |
| 200651216- | 0 | 0 | 0 | 1 | 1 | NA |
| CS_R1 | | | | | | |
| 2095639- | 0 | 0 | 0 | 1 | 1 | NA |
| CS_R1 | | | | | | |
| 2106451- | 0 | 0 | 0 | 1 | 1 | NA |
| CS_R1 | | | | | | |
| 2121694- | 1 | 0 | 0 | 1 | 1 | NA |
| CS_R1 | | | | | | |
| 2148110- | 0 | 0 | 0 | 0 | 1 | NA |
| CS_R1 | | | | | | |
| 2185127- | 0 | 0 | 0 | 0 | 1 | NA |
| CS_R1 | | | | | | |
| 2191913- | 0 | 0 | 0 | 1 | 1 | NA |
| CS_R1 | | | | | | |
| 4013825- | 0 | 0 | 0 | 0 | 0 | NA |
| CS_R1 | | | | | | |
| 6043029- | 0 | 0 | 0 | 1 | 1 | NA |
| CS_R1 | | | | | | |
| V6014- | 0 | 0 | 0 | 0 | NA | NA |
| CS_R1 | | | | | | |
| V6015- | 0 | 0 | 0 | UNK | NA | NA |
| CS_R1 | | | | | | |
| 1053432- | 0 | 0 | 0 | 0 | 1 | minor |
| CR_R1 | | | | | | 10 |
| 1059770- | 0 | 0 | 0 | 0 | 1 | minor |
| CR_R1 | | | | | | 10 |
| 1150864- | 0 | 0 | 0 | 1 | 1 | minor |
| CR_R1 | | | | | | 10 |
| 2002623- | 1 | 0 | 0 | 1 | 1 | minor |
| CR_R1 | | | | | | 10 |
| 2003068- | 0 | 0 | 0 | 0 | 1 | minor |
| CR_R1 | | | | | | 10 |
| 200439709- | 1 | 0 | 0 | 1 | 1 | minor |
| CR_R1 | | | | | | 10 |
| 200528369- | 0 | 0 | 1 | 1 | 1 | minor |
| CR_R1 | | | | | | 10 |
| 2028781- | 0 | 0 | 0 | 1 | 1 | minor |
| CR_R1 | | | | | | 10 |
| 2074289- | 0 | 0 | 0 | 0 | 1 | minor |
| CR_R1 | | | | | | 10 |
| 2183640- | 0 | 0 | 0 | 0 | 1 | minor |
| CR_R1 | | | | | | 10 |
| 3020211- | 0 | 0 | 0 | 0 | 1 | higher |
| CR_R1 | | | | | | 10 |
| 7012328- | 0 | 0 | 0 | 1 | 1 | minor |
| CR_R1 | | | | | | 10 |
| V5039- | 0 | 0 | 0 | 1 | 1 | higher |
| CR_R1 | | | | | | 10 |
| V5225- | 0 | 0 | 0 | 0 | 0 | higher |
| CR_R1 | | | | | | 10 |
| V5422- | 0 | 0 | 0 | 0 | 1 | minor |
| CR_R1 | | | | | | 10 |
| V5446- | 0 | 0 | 0 | 0 | 1 | minor |
| CR_R1 | | | | | | 10 |
| V5464- | 1 | 0 | 0 | 1 | 1 | minor |
| CR_R1 | | | | | | 10 |
| V5523- | 0 | 0 | 0 | 1 | 1 | minor |
| CR_R1 | | | | | | 10 |
| V5667- | 1 | 0 | 0 | 1 | 1 | minor |
| CR_R1 | | | | | | 10 |
| V5707- | 1 | 0 | 0 | 0 | 1 | minor |
| CR_R1 | | | | | | 10 |
| V5718- | 0 | 0 | 0 | 0 | 1 | minor |
| CR_R1 | | | | | | 10 |
| V5743- | 0 | 0 | 0 | 0 | 1 | minor |
| CR_R1 | | | | | | 10 |
| V5765- | 0 | 0 | 0 | 1 | 1 | minor |
| CR_R1 | | | | | | 10 |
| V5777- | 1 | 0 | 0 | 1 | 1 | higher |
| CR_R1 | | | | | | 10 |
| V5781- | 0 | 0 | 0 | 1 | 1 | minor |
| CR_R1 | | | | | | 10 |
| V5795- | 0 | 0 | 0 | 1 | 1 | minor |
| CR_R1 | | | | | | 10 |
| V5796- | 1 | 0 | 0 | 1 | 1 | minor |
| CR_R1 | | | | | | 10 |
| V5810- | 0 | 0 | 0 | 1 | 1 | minor |
| CR_R1 | | | | | | 10 |
| V5812- | 0 | 0 | 0 | 1 | 1 | minor |
| CR_R1 | | | | | | 10 |
| V5821- | 0 | 0 | 0 | 1 | 1 | higher |
| CR_R1 | | | | | | 10 |
| V5831- | 0 | 0 | 0 | 1 | 1 | minor |
| CR_R1 | | | | | | 10 |
| V5856- | 0 | 1 | 0 | 1 | 1 | minor |
| CR_R1 | | | | | | 10 |
| V5860- | 0 | 0 | 0 | 1 | 1 | minor |
| CR_R1 | | | | | | 10 |
| V5867- | 0 | 0 | 0 | 0 | 1 | minor |
| CR_R1 | | | | | | 10 |
| V5875- | 0 | 0 | 0 | 0 | 1 | minor |
| CR_R1 | | | | | | 10 |
| V5878- | 0 | 0 | 0 | 0 | NA | minor |
| CR_R1 | | | | | | 10 |
| V5882- | 0 | 0 | 0 | 1 | 1 | higher |
| CR_R1 | | | | | | 10 |
| V5890- | 0 | 0 | 0 | 0 | 1 | minor |
| CR_R1 | | | | | | 10 |
| V5901- | 0 | 0 | 0 | 0 | 1 | minor |
| CR_R1 | | | | | | 10 |
| V5928- | 1 | 0 | 0 | 0 | 1 | higher |
| CR_R1 | | | | | | 10 |
| V5929- | 0 | 0 | 0 | 1 | 1 | minor |
| CR_R1 | | | | | | 10 |
| V5935- | 0 | 0 | 0 | 0 | NA | minor |
| CR_R1 | | | | | | 10 |
| V5954- | 0 | 0 | 0 | 1 | 1 | minor |
| CR_R1 | | | | | | 10 |
| V5968- | 0 | 0 | 1 | 0 | 1 | minor |
| CR_R1 | | | | | | 10 |
| V5969- | 0 | 0 | 0 | 1 | 1 | higher |
| CR_R1 | | | | | | 10 |
| V5970- | 0 | 0 | 0 | 0 | 1 | minor |
| CR_R1 | | | | | | 10 |
| V5971- | 0 | 0 | 1 | 1 | 1 | minor |
| CR_R1 | | | | | | 10 |
| V5972- | 0 | 0 | 0 | 1 | 1 | minor |
| CR_R1 | | | | | | 10 |
| V5973- | 0 | 0 | 0 | 1 | 1 | minor |
| CR_R1 | | | | | | 10 |
| V5979- | 0 | 0 | 0 | 1 | 1 | minor |
| CR_R1 | | | | | | 10 |
| V5980- | 0 | 0 | 0 | 1 | 1 | minor |
| CR_R1 | | | | | | 10 |
| V5985- | 0 | 1 | 0 | 1 | 1 | higher |
| CR_R1 | | | | | | 10 |
| V5986- | 0 | 0 | 0 | 0 | 1 | minor |
| CR_R1 | | | | | | 10 |
| V5991- | 0 | 0 | 0 | 1 | 1 | minor |
| CR_R1 | | | | | | 10 |
| V6002- | 0 | 0 | 0 | UNK | NA | higher |
| CR_R1 | | | | | | 10 |

They performed shotgun whole genome metagenome (WGM) sequencing of rectal swabs, leading to >16 million short DNA sequence reads per sample. These analyses highlighted a specific microbial signature in CRPC patients compared to the control cohort, with 33 species specifically enriched in CRPC and 10 species enriched in HSPC microbiota (FIG. 4A). The inventors did not observe overall differences in the diversity indices of the two cohorts (FIG. 14C). Interestingly, in the CRPC cohort, the inventors observed an enrichment of members of the Ruminococcus and Bacteroides genera as compared to HSPC patients, mirroring the analysis performed in the murine model (FIG. 14, D and E). Among the species enriched in the hCRPC microbiota, the inventors found species belonging to the Bacteroides genus and 2 species belonging to the Ruminococcus genus, whereas in the hHSPC microbiota they found 4 commensal species belonging to the Prevotella genus. In addition to this, both S. intestinalis and D. massiliensis were found over-represented in patients with CRPC when compared to HSPC (FIG. 4, A and B).

The commensal bacteria that were most significantly associated with a worse clinical outcome (defined on patient survival from the day of the swab, for 14 months), independent of disease stage, were Ruminococcus sp. DSM_100440, Ruminococcus sp. OM05_10BH, Streptococcus vestibularis and Clostridiales bacterium VE202_14 (unfavorable species: RRSC) (FIG. 15, A-D), whereas Prevotella sp. BCRC_81118, Prevotella sp. Marseille_P4119 and Prevotella sp. 885 were associated with a more favorable outcome (favourable species: PPP) (FIG. 15, E-G). Moreover, patients with the simultaneous presence of the RRSC species (unfavorable fingerprint) had worse survival compared to the patients where the four species were not simultaneously present (FIG. 15H). In contrast, patients with the concomitant presence of the PPP species (favorable fingerprint) were associated with improved survival (FIG. 15I). Interestingly, only a minority of the patients that were still alive at follow-up (HSPC 9.21% and CRPC alive 24.32%) had this unfavorable fingerprint, with the favorable microbiota fingerprint associating with more patients alive after 14-months of follow up (FIG. 4C, FIG. 15J). In the CRPC patient cohort, Ruminococcus sp. DSM_100440 and Clostridiales bacterium VE202_14 were still associated with a poor clinical outcome (p<0.01), whereas Prevotella sp. 885 was linked to a favorable prognosis (p<0.1). Next, the inventors stratified mCRPC patients according to different ADT regimens (Abiraterone vs Enzalutamide) and they found that patients treated with Enzalutamide but not Abiraterone presented an expansion of Ruminococcaceae family (FIG. 16, A and B). KEGG pathway analysis performed on HSPC and CRPC patients' gut microbiota showed that the steroid hormone biosynthesis pathway was enriched in the microbiota of patients with CRPC (FIG. 4D).

To assess whether the species enriched in the gut microbiota of the CRPC patients were capable of synthesizing androgens, the inventors cultured in vitro 9 bacterial species enriched in the CRPC microbiota (Dysgonomonas mossii, Ruminococcus sp DSM_100440, Streptococcus vestibularis, Drancourtella massiliensis, Parasutterella excrementihominis, Sellimonas intestinalis, Lactobacillus paracasei, Campylobacter hominis, Asaccharobacter celatus) and 2 species enriched in the HSPC microbiotas (Prevotella stercorea, Actinomyces ihuae). These bacteria were incubated with a panel of metabolites of the androgen biosynthesis pathway and the conversion to downstream metabolites was monitored, following the same experimental scheme employed for the murine species (FIG. 11E). Only Ruminococcus sp. DSM_100440, enriched in CRPC microbiota, had the ability to transform pregnenolone and hydroxypregnenolone into downstream androgenic steroids, including DHEA and testosterone (FIG. 4, E and F). Importantly, Ruminococcus DSM_100440 was more abundant in fecal samples of patients having higher (>10 ng/dL, 3/4, 75%) than lower serum testosterone levels; χ² (1, n=15)=2.7841, p=0.095205 (FIG. 16C). The inventors next used the C.M. of R. sp DSM_100440 incubated with pregnenolone to treat, two patient derived-organoids (PDO), CP50 and CP50C that are respectively sensitive and insensitive (due to the presence of ARsv) to androgen-deprivation (FIG. 17A). While R. sp DSM_100440 C.M, stimulated the transcription of AR target genes in CP50, it did not in CP50C. (FIG. 17, B and C). Intriguingly, abiraterone, a selective inhibitor of CYP17A1 inhibited the bacterial conversion of pregnenolone in DHEA and testosterone (FIG. 17, D-F). RNA sequencing of R. gnavus treated with pregnenolone showed upregulation of 22 genes (log FC>3.5), some of which share high sequence homology with human CYP17 (FIG. 17, G-I). These data support the existence of a bacterial enzyme that synthesize androgenic steroids; however, further investigation is needed to identify the bacterial enzyme/s responsible for the steroid biosynthesis.

To prove that human CRPC microbiota can promote tumor growth in vivo, the inventors generated a mouse model where the gut microbiota of tumor-bearing mice were human-colonized by performing FMT experiments with feces from hHSPC or hCRPC patients. TRAMP-C1 mice pre-treated with ABX were subjected or not to CTX, received FMT, and tumor growth was then monitored (FIG. 18A and FIG. 4G). In line with the results obtained with murine FMT, hHSPC FMT limited tumor growth when compared to hCRPC FMT in CTX but not in sham-operated mice (FIG. 4G). In fact, hHSPC FMT reduced intratumoral expression of the AR target gene Fkbp5 (FIG. 18B). Notably, Ruminococcus sp. DSM_100440 could also reverse the efficacy of ABX in castrated TRAMP-C1 allograft mice when compared to Prevotella stercorea (enriched in HSPC) (FIG. 4H). As observed in the other studied mouse models, Ruminococcus sp. DSM_100440 administration increased the circulating levels of DHEA and testosterone in recipient mice (FIG. 18, C and D). Overall, these data defined a microbial blueprint in hCRPC microbiota and identified microbial species that alone or in combination impact prostate cancer outcome.

### Conclusions

The gut microbiota is a recognized player in the host's fitness, modulating numerous bioactive molecules in the intestine, blood, and various extra-intestinal organs, and may impact many cancer types through different mechanisms. However, its role in prostate cancer has remained underexplored. Some studies have reported altered fecal microbiota in PCa patients, but the mechanisms through which the microbiota impacts tumor growth have not been directly addressed. By using different mouse models of prostate cancer including PDXs, the inventors have shown that androgen deprivation (CTX, CTX+Enza) drives the expansion of a peculiar intestinal microbiota, and that the gut microbiota impacts CRPC growth by contributing to the host's androgen metabolism. The milestone at the basis of the present invention is the demonstration that these particular species can contribute to androgen metabolism, prostate cancer growth, endocrine treatment resistance, and disease outcome.

ADT is the standard first-line therapeutic strategy in lethal PCa patients, with numerous mechanisms of resistance to this treatment having been described, including increased AR expression, AR splicing, activation of aberrant cell signaling, recruitment of MDSCs or plasma cells and paracrine factors secreted by stromal cells as well as lineage plasticity. In patients treated with ADT, optimal castration is reached when patients' testosterone plasma level is reduced below 50 ng/dl. However, clinical evidence demonstrates that patients having androgens levels <32 ng/dl have better outcomes than patients with testosterone levels between 32 and 50. Thus, subtle variations in plasma androgen levels can impact the prognosis of prostate cancer patients. These findings can provide novel opportunities for the therapy of prostate cancer patients. Indeed, it was demonstrated that FMT with HS microbiota or administration of P. stercorea can decrease androgens levels in CTX mice and delay the onset of CRPC. FMT has become the first line therapy against Clostridium difficile infection, with clinical trials demonstrating its efficacy also in ulcerative colitis, and a number of ongoing clinical trials are studying this therapeutic strategy further. However, translation to transformative clinical trials in prostate cancer using FMT could be challenging, since HS patients become CR several years after starting ADT.

Moreover, the inventors identify in CRPC patients a fecal bacterial signature that associates with patients' overall survival. This signature could be used as a minimally invasive biomarker to identify patients that could benefit from microbiota manipulation strategies. Finally, these data can help drive further research focused on elucidating how microbiota fuel prostate carcinogenesis through unregulated AR signaling activation in prostatic epithelial cells.

### Example 5—Bacterial Consortia

The administration of bacterial consortium comprising P. stercorea and Lactobacilli (L. acidophilus, L. paracasei, L. buchneri and L. bulgaricus) or P. stercorea and Bifidobacterium (B. bifidum, B. longum, and B. breve) was able to control CRPC tumor growth (FIG. 19, A, B). Example 6—Treatment of Prostate Cancer with postbiotics based on P. stercorea

To determine if Prevotella stercorea could be active also in other form or formulation rather that alive in controlling tumor prostate tumor growth, experiments were performed by administering to mice batches of postbiotics represented either by inactivated Prevotella or its metabolic products. Prevotella stercorea (PV) inactivation through pasteurization (70° C., 30 min) or tindalization results in a product able to control prostate cancer tumor growth to levels comparable to alive PV and antibiotic treatment. In addition, also postbiotic of PV containing its metabolic products results in a tumor growth reduction, even if in a less efficient manner when compared to alive PV or antibiotics. The production of PV metabolic products was performed as follow: exponentially growing culture of PV were subjected to centrifugation (3500 rpm, 15 min) and the supernatant was concentrated with 3 kDa Amicon Ultra filter sistem (Millipore). The eluate was then evaporated with SpeedVac (Thermo Scientific) and the two fractions (the one containing molecules bigger then 3 kDa and the one evaporated with SpeedVac) were merged and administered to the mice by oral gavage.

To investigate the mechanism by which PV may opposes PCa progression, a panel of prostate cancer cells (LNCaP, PC3, 22RV1) was treated with conditioned media (CM) from PV or vehicle. To decrease bias effect related to the addition of bacterial culture media into an eukariotic culture systems, a cultivation method was established in which Prevotella stercorea is cultured in RPMI supplemented with 20% of FBS. Surprisingly, prostate cells treated with PV CM grow less, indicating that PV CM contains molecules that inhibit cancer cell growth (FIG. 21 A-C). Prostate cancer cell proliferation was monitored with Incucyte System for live-cell imaging analysis. These results were also validated in LNCaP cultured in full androgen deprivation (FAD) conditions (FIG. 21 D). Interestingly, PV CM could control tumor growth only in tumoral cells, but it was ineffective in normal prostate cancer cells (RWPE1) (FIG. 21E). To determine which component contained in PV CM may affect the prostate cancer cell proliferation, sequential concentration steps were performed using Amicon Ultra filter system (Millipore) ranging from 100 kDa to 3 kDa and the different fractions were administered separately to prostate cancer cells. The different fractions administered impacts differently on cancer cell proliferation, with the fraction containing protein bigger that 100 Kda having the most profound effect (FIG. 21F). The mechanism by which PV CM administration may halt prostate cancer cell proliferation was checked and it was discovered that cells exposed to PV CM display an increase in apoptotic cells when compared to vehicle (FIG. 21G).

### Materials and Methods

Cell lines. TRAMP-C1 (ATCC® CRL-2730™), PC3 (ATCC® CRL-1435™) and LNCaP (ATCC® CRL-1740™) cell lines were purchased from ATCC (no authentication method was performed), cultured following ATCC guidelines and regularly tested for mycoplasma (MycoAlert Mycoplasma Detection kit, Lonza, Cat. LT07-218). For in vitro experiments TRAMP-C1 cells were starved in charcoal-stripped FBS medium plus Enzalutamide (ENZA) 10 μM for 72 h and then kept in full androgen-deprivation medium (FAD; DMEM containing 10% heat-inactivated charcoal-stripped FBS plus ENZA 10 μM). Then, cells were stimulated for 48 h with culture broth alone or with the conditioned media (CM) obtained from exponentially growing bacterial cultures (1:8 dilution in charcoal-stripped FBS) and then collected for RNA isolation.

PDX derived organoid culture in-vitro. PDX tumours from castrated (CP50C) and intact (CP50) sublines were harvested in PDX harvesting solution (adDMEM/F12 containing 10 μM ROCK inhibitor (Selleck Chemicals, Y27632), penicillin/streptomycin, 10 mM Hepes and GlutaMAX 1× (Thermofisher), cut into small pieces (1-3 mm³) and single cell suspensions were generated by mechanical separation (40 μm Corning cell strainer, Sigma Aldrich). Pellets were washed once on ice-cold PBS/10 μM Y27632, and red blood cells were removed using red blood cell lysis buffer (0.8% NH4Cl in 0.1 mM EDTA in water, buffered with KHCO3 to pH of 7.2-7.6, incubated 1-minute on ice) followed by another wash with ice-cold PBS/Y27632. Single cell suspensions were resuspended in ice-cold organoid growth medium (as published by (J. Drost et al., Organoid culture systems for prostate epithelial and cancer tissue. Nat Protoc 11, 347-358 (2016)) with the following alterations: The p38 inhibitor SB202190 was replaced by the addition of 5 nM NRG1 and subsequently diluted in one volume of phenol red-free, growth factor reduced, Corning Matrigel™ (Fisher Scientific). Organoid domes (50 μl) were plated as previously described (J. Drost et al., Organoid culture systems for prostate epithelial and cancer tissue. Nat Protoc 11, 347-358 (2016)) and topped up with warm medium after solidification. As soon as organoid formation was confirmed by visual examination (microscope), organoids were harvested in cold PBS/Y27632, washed, resuspended in fresh organoid medium/Matrigel (1:1), and seeded in 25 μl Matrigel domes. 24 h after seeding, medium was replaced by medium supplemented with 10 μM Enzalutamide and another 72 h later, medium was replaced by conditioned medium as indicated mixed in a 1:4 ration with organoid medium/10 μM Enzalutamide.

Patient-derived xenograft and western blot. PDX tumours pieces (1-3 mm³) were subcutaneously implanted into the flanks of NSG mice. Tumours were measured using mechanic calipers and grown to the volume of 400 mm³; body weight was monitored twice weekly. When tumours reached a size of 400 mm3, mice were either castrated (CP50C) or left intact (CP50). A week after castration, tumours were harvested, shock frozen in liquid nitrogen, grinded to powder using a Qiagen tissue lyser, and lysed in RIPA lysis buffer supplemented with Protease/Phosphatase inhibitor mix (both Thermofisher Scientific) for 30 min on ice. Samples were sonicated for 10-15 strokes, centrifuged for 30 min at 13 000 rpm at 4° C. and analysed using a 4-12% BisTris NuPAGE™ gel and MES running buffer (Thermofisher) using antibodies against AR (D6F11, Cell Signalling), FKBP5 (SAB2100820, Sigma Aldrich), PSA (ab231238, Abcam), Tubulin (sc-B7, Santa Cruz), Vinculin (hVIN-1, Sigma Aldrich) and GAPDH (sc-G9, Santa Cruz).

Bacteria. Bacteroides acidifaciens (10556T) was purchased from JCM, Ruminococcus gnavus (ATCC® 29149™) and Clostridium scindens (ATCC® 35704™) were purchased from ATCC, Enterococcus faecalis (ATCC® 29212™), Enterobacter cloacae (ATCC® 13047™), Proteus mirabilis (ATCC® 12453™), Serratia Marcescens (ATCC® 43861™), Staphylococcus aereus (ATCC® 29213™), Escherichia Coli (ATCC® 25922™) were kindly shared from the Microbiology Unit of EOC (Bellinzona, Switzerland). Dysgonomonas mossii (DSM 22836), Ruminococcus sp._DSM 100440 (DSM 100440), Streptococcus vestibularis (DSM 5636), Drancourtella massiliensis (DSM 100357), Parasutterella excrementihominis (DSM 21040), Sellimonas intestinalis (DSM 103502), Lactobacillus paracasei (DSM 20312), Campilobacter hominis (DSM 21671), Adlercreutzia equolifaciens subsp. celatus (DSM 18785), Prevotella Stercorea (DSM 18206) were purchased from German Collection of Microorganism and Cell Cultures GmbH (DSMZ). Actinomyces ihuae (CSUR P2923) was purchased from IHU Mediterranée Infection. B. acidifaciens, R. gnavus, E. faecalis, E. cloacae, P. mirabilis, S. marcescens, D. mossii, R. sp.DSM 100440, S. vestibularis, D. massiliensis, P. excrementihominis, S. intestinalis, A. equolifaciens subsp. celatus, P. stercorea and A. ihuae were cultured on Columbia Agar with 5% Sheep Blood (BD, Cat. 254071) and TSB (Oxoid, Cat. CM0129) or THIO (BD, Cat. 211720) broth, C. scindens was cultured on Schaedler Agar with Vitamin K1 and 5% Sheep Blood (BD, Cat. 254084) and THIO broth. All strains were cultured in anaerobic incubator (Scholzen Microbiology Systems AG) with an atmosphere of 80% N2, 10% H2, 10% CO2; anaerobic atmosphere was confirmed with the use of BLL™ GasPack™ Anaerobic Indicator Strip (BD, Cat. 271051). L. acidophilus, L. paracasei, L. buchneri and L. bulgaricus, B. bifidum, B. longum, and B. breve were cultured following standard procedures required for these strains. To evaluate bacterial ability to metabolize androgen precursors, 0.5 ml of bacteria solution at OD600 1 McFarland was inoculated in 6.5 ml of culture media with 50 μM of pregnenolone acetate (Sigma Aldrich, 700142P), 17α-hydroxypregnenolone (Sigma Aldrich, H5002), cholesterol (Sigma Aldrich, C3045), cortisol (Sigma Aldrich—hydrocortisone-, H0888) or aldosterone (Sigma Aldrich, A9477) (readapted from (J. Winter et al., Mode of action of steroid desmolase and reductases synthesized by Clostridium “scindens” (formerly Clostridium strain 19). J Lipid Res 25, 1124-1131 (1984))). After 48 h, culture broth was collected and analyzed with targeted mass spectrometry to detect metabolic conversion. To evaluate abiraterone and abiraterone acetate ability to inhibit bacterial steroid production, 0.5 ml of bacteria solution at OD600 1 McFarland was inoculated in 6.5 ml of culture media with 50 μM of pregnenolone acetate and either vehicle (EtOH), 10 or 100 μM of abiraterone acetate (MedChemExpress, Cat.HY-75054), 10 or 100 μM of abiraterone (MedChemExpress, Cat.HY-70013). After 48 h, culture broth was collected and analyzed with targeted mass spectrometry to detect metabolic conversion.

Mice. Mice were maintained under specific pathogen-free conditions and experiments were approved by the local ethical committee (TI 32/2018). 4 weeks old male C57BL6/N and NOD/SCID mice were purchased from Charles River (Calco, Italy) and acclimatized for four weeks before experimentation. NRG mice were generated at IRB animal facility, Bellinzona, Switzerland. For allograft experiments, C57BL6/N were challenged with 2.5×106 TRAMP-C1 cells and castrated when tumors were approximately 100 mm³. For xenograft experiment, NRG mice were challenged with 2.5×10⁶ PC3 cells or 2.5×10⁶ LNCaP cells in matrigel (Corning®, Cat. 356231) and castrated when tumors were approximately 100 mm³. For patient-derived xenograft (PDX), NRG mice were challenged with 2.5×10⁶ LuCaP-145.2, -35 (H. M. Nguyen et al., LuCaP Prostate Cancer Patient-Derived Xenografts Reflect the Molecular Heterogeneity of Advanced Disease an--d Serve as Models for Evaluating Cancer Therapeutics. The Prostate 77, 654-671 (2017)). PDXs were provided from Jean-Philippe Theurillat Lab. Briefly, PDXs tumors were maintained by subcutaneous implantation of matrigel-embedded tumor fragments (1-2 mm³ average diameter). PDX tumor tissue was cut into small pieces (1-0.5 mm) with a scalpel blade and then digested in Collagenase Type I media solution (200 U/ml Millipore, Cat.SCR103) at 37° C. for 45-60 min. After enzymatic dissociation, the cell suspension was passed through a 100 μM cell strainer (Roche, 11814389001) to eliminate macroscopic tissue pieces and then centrifuged. The cell pellet was then resuspended in 2-volume RBC lysis buffer (Roche, 11814389001), incubated for 3 min at RT, washed and centrifugated. Cells were resuspended in PBS and 50% matrigel (Corning®, Cat.356231) and subcutaneously injected. The tumor volume was calculated as 4/3π(R1×R2×R3), where R1 and R2 are the longitudinal and lateral radii and R3 is the thickness of the tumor that protrudes from the surface of normal skin (A. Calcinotto et al., IL-23 secreted by myeloid cells drives castration-resistant prostate cancer. Nature 559, 363-369 (2018)). Male Pten^{pc-/-} mice were generated and genotyped as previously described (A. Calcinotto et al., IL-23 secreted by myeloid cells drives castration-resistant prostate cancer. Nature 559, 363-369 (2018)) and castrated at 9-10 weeks of age. Surgical castration was performed under anesthesia with isoflurane as previously described (A. Calcinotto et al., IL-23 secreted by myeloid cells drives castration-resistant prostate cancer. Nature 559, 363-369 (2018)). To deplete the intestinal microbiota, mice were treated with a cocktail of neomycin (1 g/L), ampicillin (1 g/L), and vancomycin (0.5 g/L) in the drinking water and were daily administered with 2 mg metronidazole per os (F. Fransen et al., BALB/c and C57BL/6 Mice Differ in Polyreactive IgA Abundance, which Impacts the Generation of Antigen-Specific IgA and Microbiota Diversity. Immunity 43, 527-540 (2015)). For fecal microbiota transplantation (FMT) experiments, mice were treated with ABX for 7 days before CTX and then received FMT for three consecutive days on the first week and once a week for the following weeks. Murine fecal material was collected from donor mice, resuspended at 50 mg/ml in sterile PBS, and administered via oral gavage 200 μl/mouse. For human FMT, selected Fecal Swabs (FecalSwab™, Copan) were thawed on ice, and media containing bacteria was mixed and centrifuged at 3200 g. The pellet containing bacteria was resuspended in Columbia broth (BD Difco™, Cat.294420) 25% glycerol pre-incubated in anaerobic atmosphere. Bacterial suspensions were aliquoted and frozen at −80° C. in cryo-vials (Corning®, Cat.430488). For human FMT administration, frozen aliquots were thawed on ice, centrifuged at 3200 g, and resuspended in sterile PBS pre-incubated in anaerobic atmosphere. In bacterial administration experiments, TRAMP-C1 allografts were treated for 7 days with ABX before CTX. After, mice were administered with 10⁹ CFUs of exponentially growing cultures of B. acidifaciens, R. gnavus, R. sp DSM100440, and P. stercorea.

To track the conversion of ^{D}pregnenolone from the intestinal microbiota, CTX Pten^{pc-/-} animals both in CS and CR phase either untreated or ABX treated were injected i.v. with 75 ng/mouse pregnenolone sulfate sodium salt (20,21-¹³C₂, 99%; 16,16-D2, 98%) (Cambridge Isotope Laboratories, Inc; Cat. CDLM-9160-0.001). Serum and feces were collected at 0, 2, 6, 12 and 24 hours post-injection and analyzed by LC-MS/MS to detect downstream metabolites.

For αLy6G treatment, mice were injected intraperitoneally with 64.9 μg of InVivoPlus anti-mouse Ly6G (BioXCell, Cat. BP0075-1) 3 times per week. Enzalutamide (APExBio, cat.MDV3100) was administered daily by oral gavage with a dose of 30 mg/kg per day on a Monday through Friday schedule. Testosterone (HANSELER, cat. 06-8202-02) was diluted in corn oil (Sigma-Aldrich, cat.8267) and was intraperitoneally administered (25 mg/kg) on a Monday through Friday schedule. Testosterone hematic levels upon treatment were checked with Testosterone ELISA kit, (Abcam, AB108666). At the endpoint, mice were euthanized by CO₂ asphyxiation, and tissues were collected for histology, mRNA isolation, and flow cytometry experiments.

Magnetic Resonance Imaging. Magnetic resonance imaging (MRI) study was performed on Pten^{pc-/-} mice surgical castrated or sham-operated either untreated or treated with ABX cocktail at 10, 13, 16 and 20 weeks using a 7T preclinical scanner (Bruker, BioSpec 70/30 USR, Paravision 6.0.1), equipped with 450/675 mT/m gradients (slew-rate: 3400-4500T/m/s; rise-time 140 μs) and an inner diameter of 40 mm, with a circular polarized mouse body volume coil. All mice underwent imaging under inhalational anesthesia (Isoflurane, 3% for induction and 2% for maintenance in 2 L/minute oxygen), lying prone on a dedicated temperature-controlled apparatus to prevent hypothermia, with breathing rate and body temperature continuously monitored (SA Instruments, Inc., Stony Brook, NY, USA). MRI protocol included: Rapid Acquisition with Relaxation Enhancement (RARE) T2-weighted sequence in axial plane and in coronal planes (slices 22, thickness 0.70 mm, gap=0, TR/TE=3500/40 ms, MTX=256×190, FOV=30×20 mm), the axial images were acquired both with and without fat suppression, while the coronal images only without fat suppression; Multi-Slice-Multi-Echo (MSME) sequence acquired on axial plane slices; thickness=0.7 mm; gap=0; TR=3200 ms Echo Images=20 and TE=from 6.78 to 135.63 ms, MTX=128×128, FOV=30×20 mm) and Diffusion map EPI sequence (TR/TE=3000/23 ms, MTX=128×128, FOV=30×20 mm, B value=30, 400, 600, 800, 1000; 14 slice). Images were analyzed using NIH software MIPAV (version 7.4.0). The prostate volume has been calculated drawing a region of interest (ROI) along the prostate border in each RARE T2w axial slice, the number of bounded pixels in each slice was computed and added to yield the prostate volume. Coronal T2w images were used for accurate identification of the basal and apical limits of the prostate.

RNA extraction, RT and qPCR. Upon necroscopy, anterior prostate (AP) lobes from Pten^{pc-/-} mice or portions of tumors from TRAMP-C1 allografts were snap-frozen. For RNA extraction tumors were disrupted with disposable pestels in 500 μl TRIzol (Invitrogen). Then RNA was purified by extraction with 100 μl chloroform and precipitation of the aqueous phase with one volume of 100% ethanol. RNA was further purified with RNeasy Mini Kit (Qiagen). Retro-transcription (RT) reaction was done with ImProm-II reverse transcriptase kit (Promega, Cat. A3800). qPCR assays were performed with GoTAQ® qPCR Master Mix (Promega, Cat. A6002). Biorad primers used were Hprt PrimePCR PreAmp for SYBR Green Assay (Hprt, mouse qMmuCID0005679), Ar PrimePCR PreAmp for SYBR Green Assay (Ar, mouse qMmuCID0005164), Pbsn PrimePCR PreAmp for SYBR Green Assay (Pbsn, mouse qMmuCID0017831), Fkbp5 PrimePCR PreAmp for SYBR Green Assay (Fkbp5, mouse qMmuCID0023283). Primer sequences for Nkx3.1, R18s, Aldh1a3, and Ppap2a are listed in

TABLE 3
Primers used for qPCR analyses

| | | | SEQ ID |
|---|---|---|---|
| Gene | Direction | Sequence 5'-3' | Number |
| NKX3.1 | Forward | TCCGTCTTTTGGCTCTGAGT | 1 |
| | Reverse | GTGAAAGTGCACGCTGAAAA | 2 |
| R18S | Forward | ACCGCAGCTAGGAATAATG | 3 |
| | Reverse | GCCTCAGTTCCGAAAACCA | 4 |
| ALDH1A3 | Forward | GGGTCACACTGGAGCTAGGA | 5 |
| | Reverse | CTGGCCTCTTCTTGGCGAA | 6 |
| PPAPA2A | Forward | AGAGGGGCTTTTTCTGTACTGA | 7 |
| | Reverse | TATACGGGACGGGATGGTACT | 8 |
| R. gnavus | Forward | TGGGTGTAAAGGGAGCGTAG | 9 |
| | Reverse | CTCTCCGACACTCTAGCCTG | 10 |
| B. acidifaciens | Forward | TTCGGTATGGGATGGGGATG | 11 |
| | Reverse | CATCCTTCACGCTACTTGGC | 12 |
| EUB16S | Forward | GGTGAATACGTTCCCGG | 13 |
| | Reverse | TACGGCTACCTTGTTACGA | 14 |

Expression levels were normalized to the expression of the housekeeping gene HPRT for in vivo experiments and to the housekeeping gene R18S for in-vitro experiments. For PDOs experiment: RNA was extracted 24 h after stimuli using the RNAesy miniprep kit (Qiagen), reverse transcribed using RevertAid™ reverse transcriptase with random primers (Thermofisher), and analysed on the ViiA 7 Real-Time PCR System (Applied Biosciences) using Taqman probes (Thermofisher) detecting RNA for AR (Hs00171172), PSA (Hs00428384), FKBP5 (Hs01561003), PMEPA (Hs00375306), NKX3.1 (Hs00171834) using RPLPO (Hs00420895) as a reference to calculate 2\hat{ }-ddCT. Results of conditioned medium treated samples were normalized to matching control medium to calculate fold expression.

Bacterial RNA sequencing. Exponentially growing culture of Ruminococcus gnavus were inoculated either with vehicle (EtOH) or with 50 μM pregnenolone (Sigma Aldrich, 700142P) for 1 hour. Bacterial cultures were then stabilized with RNAprotect Bacteria Reagent (Qiagen, cat. 76506), then enzymatic lysis (15 mg/ml lysozyme, Sigma Aldrich, cat.L6876) and proteinaseK (Qiagen, cat.19131) digestion were performed following manufacturers' guidelines. RNA was then extracted using RNeasy Mini Kit (Qiagen, cat.74106) and on-column DNase treatment was performed following manufacturers' guidelines. RNA integrity was checked by agarose gel electrophoresis. Illumina Stranded Total RNA with Ribo Zero Plus (Illumina, San Diego, CA, USA) was employed with IDT® for Illumina® RNA UD Indexes Set A, (Illumina, San Diego, CA, USA) for cDNA synthesis and addition of barcode sequences. Sequencing of the libraries was performed using the NextSeq 500 (Illumina, San Diego, CA, USA) with the NextSeq 500/550 High Output Kit v2 (75 cycles; Illumina). Samples were processed starting from stranded, single-ended 75 bp-long sequencing reads. Fastq files were generated using BaseSpace tool (Illumina, San Diego, CA, USA). Ruminococcus gnavus ATCC 29149 genome assembly ASM16947v1 was retrieved from EnsemblBacteria (https://bacteria.ensembl.org) along with the respective gene annotations. Genome indexes and the subsequent sequencing read alignment were performed using bwa-mem. For all samples, more than 70% of the reads could be mapped to the reference genome. Counts per gene were quantified using featurecounts through meta-feature summarization, and single-end reads were considered as being reversely stranded (−s 2 option). Raw counts were imported in R statistical environment. Library size normalization and differential expression analysis between pregnenolone exposed (1 h) and untreated controls were performed using DESeq2 pipeline.

Histology/histopathology. Upon necroscopy, AP lobes from Pten^{pc-/-} mice or portions of tumors from TRAMP-C1 allografts were fixed in 10% formalin (5701, ThermoScientific) and paraffin-embedded. Preceding immunohistochemical staining, tumour sections (4 μm) were exposed to two washes with OTTIX plus solution (X0076, Diapath) and subsequent hydration with OTTIX shaper solution (X0096, Diapath) followed by deionized water. Antigen unmasking was performed by heating sections at 98° C. for 20 min in solutions at specific pH, according to the antibodies used. Subsequently, the sections were incubated for 10 min with 3% H2O2 (23615.248, VWR) to quench endogenous peroxidases, washed in 0.5% PBST, and incubated for further 10 min in Protein-Block solution (X0909, DAKO Agilent technologies) to block non-specific antibody binding. Hematoxylin and eosin staining was performed according to standard procedures. Sections were stained for anti-Ki-67 (clone SP6; Lab Vision Corporation, RM-9106-R7) and anti-cleaved Caspase 3 (Cell Signaling, 9661). Sections were further incubated with Anti-Rabbit secondary antibody (Vector laboratories, cat. BP-9100). Sections were then incubated with Vectastain ABC (Vector, Cat. PK-6100) for 30 min and then with ImmPACT DAB peroxidase (HRP) substrate (Vector, Cat. SK-4105) for 3-4 min. Immediately, slides were washed 3 times with PBST and counterstaining was performed using hematoxylin solution (Diapath, Cat. C0303). At the end of IHC staining, sections were dehydrated using deparaffinization procedure after which slides were mounted with coverslip using aqueous mounting media (Diapath, Cat. 060200). Images were obtained with Aperio ScanScope, Leica Biosystem. All the quantifications were performed with ImageScope, v12.3.2.8013, Leica Biosystem by acquiring at least 10 glands per sample. Positive signals in epithelial tissue were selected for analysis. For histopathological characterization of the prostate tumors of Pten^{pc-/-} mice, hematoxylin and eosin stained sections of prostate tumors of indicated genotype/treatment were assessed for normal-like, prostatic intraepithelial neoplasia (PIN), adenocarcinoma, and invasive carcinoma.

Untargeted Metabolomic analysis. Metabolome extraction, purification, and derivatization were carried by the MetaboPrep GC kit (Theoreo, Montecorvino Pugliano, Italy) according to manufacturer instructions. Instrumental analyses were performed with a GC-MS system (GC-2010 Plus gas chromatograph and QP2010 Plus mass spectrometer; Shimadzu Corp., Kyoto, Japan) as described by Troisi et al. (J. Troisi et al., A metabolomics-based approach for non-invasive screening of fetal central nervous system anomalies. Metabolomics 14, 77 (2018), J. Troisi et al., Metabolomic Signature of Endometrial Cancer. J Proteome Res 17, 804-812 (2018)). Metabolite identification was performed according to Troisi et al. (see above), the linear index difference max tolerance was set to 10, while the minimum matching for NIST library search was set to 85% (level 2 identification according to Metabolomics Standards Initiative [MSI])(L. W. Sumner et al., Proposed minimum reporting standards for chemical analysis Chemical Analysis Working Group (CAWG) Metabolomics Standards Initiative (MSI). Metabolomics 3, 211-221 (2007)). Metabolites that emerged as the most relevant in separating cases from controls were further confirmed using external standards (MSI level=1).

Targeted metabolomic analysis. Hormone extractions were based on Supported Liquid Extraction (SLE) using the Novum SLE cartridge (Phenomenex, Milan, Italy). 100 μl of each liquid sample, or 50 mg of feces, were diluted with 100 μL of water and loaded into each well of a Novum SLE MINI 96-Well Plate after internal standard spiking. 5 mmHg negative pressure was applied for 10 seconds, then left five minutes without vacuum. 1 mL of 90:10 dichloromethane/ethanol was added and eluted by gravity flow, then the elution was completed with further 10 second of 5 mmHg negative pressure. Solvent was blown down with a gentle stream of nitrogen at 40° C. The extracts were reconstituted with 100 μL of Acetonitrile/Water 20/80 and vortexed for 5 minutes at 1250 rpm. UHPLC-MS/MS analysis was carried out with a Shimadzu Nexera (Shimadzu, Milan, Italy) UHPLC consisting of two LC 30 AD pumps, a SIL 30AC autosampler, a CTO 20AC column oven, a CBM 20 A controller, and the system was coupled online to a triple quadrupole LCMS 8050 (Shimadzu, Kyoto, Japan) by a ESI source. Metabolite separation was achieved on a Kinetex Biphenyl 100A 100×2.1 mm×2.6 μm (Phenomenx, Milan, Italy) at a flow rate of 500 μL/min, employing as mobile phase A) water 5 mM HCOONH4 and B) ACN with the following gradient: starting 0 min, 2% B, 0.01-5.00 min, 100% B, 5.01-6.50 min, isocratic at 100% B. Returning to 2% in 5 min. 5 μl were injected. All additives and mobile phases were LC/MS grade and purchased from Sigma Aldrich (Milan, Italy). The ESI was operated in positive ionization. MS/MS analysis were conducted in multiple reaction monitoring (MRM) using at least 2 transitions for quantification and confirmation. The MS/MS analyses were performed setting the following parameters: Interface temperature 300° C., desolvation line temperature 200° C., heat block temperature 400° C.; nebulizing gas, drying gas and heating gas were set respectively to: 3, 10, and 10 L/min. The instrumental calibration was performed through the external standard method. Stock solution was prepared and diluted to obtain the calibration standard in a concentration range between 100-2.5 ng/mL. Six concentration levels and triplicate injection of each level were run.

Pten^{pc-/-} Murine 16S rDNA sequencing analysis and bacteria qPCR. 16S rDNA sequencing analysis was performed exclusively on murine samples. Murine fecal pellets were snap-frozen and stored at −80° C. until processing. DNA was extracted using a GNOME DNA isolation kit (MP) following the protocol described in (J. P. Furet et al., Comparative assessment of human and farm animal faecal microbiota using real-time quantitative PCR. FEMS Microbiol Ecol 68, 351-362 (2009)). Microbial DNA, V1-V3 hypervariable region of the 16S rRNA gene, was amplified and sequenced as previously described (S. Tomkovich et al., Locoregional Effects of Microbiota in a Preclinical Model of Colon Carcinogenesis. Cancer Res 77, 2620-2632 (2017)). In short, DNA concentration and quality were evaluated using NanoDrop 2000. The V1-V3 hypervariable region of the 16S rRNA gene was amplified using the primer pair 8F (5′-AGAGTTTGATCCTGGCTCAG-3′) (SEQ ID N 15) and 534R (5′-ATTACCGCGGCTGCTGG-3′) (SEQ ID N 16). Both the forward and reverse primers contained universal Illumina paired-end adapter sequences, as well as unique individual 4-6 nucleotide barcodes between the PCR primer sequence and the Illumina adapter sequence to allow multiplex sequencing. PCR products were visualized on an agarose gel then quantified by qPCR using the KAPA Library Quantification Kit (KAPA Biosystems, KK4824). Equimolar amounts of samples were then pooled. The pooled library was purified using the Agencourt AMPure XP kit (Beckman Coulter, A63880) and quantified with the same method prior to sequence using paired-end (read length=300 bases) on Illumina MiSeq. 16S sequencing reads were preprocessed using QIIME v.1.9.1 (J. G. Caporaso et al., QIIME allows analysis of high-throughput community sequencing data. NatMethods 7, 335-336 (2010)) with the default parameters. Briefly, forward and reverse reads were merged, demultiplexed and quality filtered at Q20. Open-reference OTUs were picked at 97% similarity level using the Greengenes 97% reference dataset, release 13_8 and chimeric sequences were detected and removed using QIIME. Taxonomy was assigned using RDP (ribosomal database project) classifier (J. G. Caporaso et al., QIIME allows analysis of high-throughput community sequencing data. Nat Methods 7, 335-336 (2010)) v. 2.2 through QIIME also using Greengenes release 13_8 reference sequences with confidence set to 50%. We excluded OTUs that had ≤0.005% of the total number of sequences according to Bokulich et al. (N. A. Bokulich et al., Quality-filtering vastly improves diversity estimates from Illumina amplicon sequencing. NatMethods 10, 57-59 (2013)).

The resulting OTU counts were normalized and log 10 transformed using the following formula:

$${log}_{10} ( \frac{RC}{n} \times \frac{\sum x}{N} + 1 )$$

where RC is the read count for a particular OTU in a particular sample, n is the total number of reads in that sample, the sum of x is the total number of reads in all samples and N is the total number of samples. The Principle Coordinate Analysis (PCoA) was generated from the Bray-Curtis distance of the normalized and log 10 transformed counts using the phyloseq R package. Alpha diversity measures (Observed OTU, Chao1 and Shannon) were also calculated after rarefying the raw counts to a depth of the minimum count in all samples. We utilized the nlme package v. 3.1-131 in R v. 3.4.3 to analyze the data and account for possible contributions that may arise from co-housing groups of mice in the same cage. We built two models: one with cage modeled as a random effect and treatment as fixed effects, and one without cage. We then used ANOVA to compare the two models and the resulting P-value (calculated using F-test) was used to determine the effect of co-housing. The treatment P-values were calculated using ANOVA on the mode that does not include cage. We controlled for false discovery rate (FDR) by correcting the P-values using the Benjamini and Hochberg (BH) approach (Y. Benjamini, Y. Hochberg, Controlling the False Discovery Rate: A Practical and Powerful Approach to Multiple Testing. Journal of the Royal Statistical Society: Series B (Methodological) 57, 289-300 (1995). For taxa analyses (phylum, class, order, family and genus), the OTUs were collapsed to the desired taxon based on their RDP classification before analyzed as described above. R. gnavus and B. acidifaciens abundance was detected using qPCR assay with specific primers and normalized to EUB16S (primers listed in table 3).

Cytokine array. To detect circulating cytokine abundance serum from CTX Pten^{pc-/-} mice treated or not with ABX in CR phase were analyzed with Rodent MAP 4.0-Mouse (Ampersand Biosciences). When values were below the least detectable dose (LDD) of the technique, the LDD value was reported.

Flow cytometry. To obtain single cell suspensions we processed the different organs as follows. Tumors were cut in small pieces with scissors, digested in collagenase D (5 μg/ml, Roche, Cat. 11088858001) and DNase (20 μg/ml, Roche, Cat.4716728001) for 30 min at 37° C. and smashed onto a cell strainer (Falcon®, Cat.352340). To obtain bone marrow cells, femurs were flushed with ice-cold PBS and red blood cells were lysed with ACK buffer (Gibco, Cat. A10492-01). Spleens were smashed onto a cell strainer and red blood cells lysed with ACK buffer. Peyer's Patches and mesenteric lymph nodes were smashed on cell strainers. Colons were collected in ice-cold PBS and incubated for 20 min at 37° C. on spinning wheel in DMEM 1% FBS 1% P/S 1 mM EDTA. The resulting fraction was kept as intra-epithelial lymphocytes (IELs). The remaining chunks were further digested in DMEM 1% FBS 1% P/S with collagenase D (5 μg/ml) and DNase I (10 μg/ml) for 30 min at 37° C. on spinning wheel and after smashed onto cell strainers. This fraction contains lamina propria (LP) cells. Samples obtained in these ways were blocked for Fc receptor binding with CD16/CD32 antibody (clone 93) for 15 min, then stained with the antibodies listed in Table 4. Samples were acquired at BD Fortessa cytometer (BD Biosciences). Data were analyzed using FlowJo software (LLC). For gating, isotype controls or fluorescence-minus-one controls were used.

TABLE 4
Antibodies used for FACS analyses.

| | Company | Clone | Code | Dilution |
|---|---|---|---|---|
| F4/80 | Biolegend | BM8 | 123120 | 1/200 |
| CD4 | Biolegend | GK1.5 | 100434 | 1/200 |
| Ly6C | Invitrogen | Hk1.4 | 45-5932-82 | 1/200 |
| NK1.1 | Invitrogen | PK136 | 17-5941-82 | 1/200 |
| CD3 | Biolegend | 145-2C11 | 100329 | 1/200 |
| CD90.2 | Biolegend | 30-H12 | 105341 | 1/200 |
| CD11b | Biolegend | M1/70 | 101236 | 1/200 |
| CD45 | Biolegend | 30-F11 | 103139 | 1/200 |
| CD8 | Invitrogen | 53-6.7 | 12-0081-83 | 1/200 |
| CD11c | BD | HL3 | 553802 | 1/200 |
| CD19 | Biolegend | 6D5 | 115520 | 1/200 |
| Ly6G | Biolegend | 1A8 | 127618 | 1/200 |
| CD16/32 | Biolegend | 93 | 101302 | 1/50 |

Collection of patient feces. Patients with hormone-sensitive (HSPC) and metastatic CRPC consented to the analysis of their rectal swabs. Consent was obtained within a Royal Marsden Hospital (RMH) specific protocol (Ethics Review Committee reference no. 04/Q0801/60) and within Ente Ospedaliero Cantonale (EOC) specific protocol (IOSI-IOR 002, 2017-01631, CE TI 3269). Samples were collected using FecalSwab™ (Copan) and immediately stored at −80° C. This study was conducted in accordance with the Declaration of Helsinki. Demographic and clinical data for each patient were collected from the hospital electronic patient record. Patients' characteristics are reported in Table 1 and Supplementary Table 1.

Whole genome metagenome analysis of patient feces. Whole genome metagenome analysis was performed exclusively on patients' feces. DNA was isolated with QIAamp Fast DNA Stool Mini Kit (Qiagen, 51604) with the addition of a bead beating step: 180-220 mg stool samples were added to 70° C. prewarmed 1 ml InhibitEX® buffer (Qiagen, cat.19593) containing 600-700 mg 0.1 mm glass beads. The suspension was heated for 5 min at 95° C.±3° C. while shaking at 1000-1200 rpm. Samples were then homogenized with FastPrep-24 5G (cat.116005500) 4×45 s at speed 4. Samples were centrifugated 5 min. at 20'000 g, and supernatant was transferred to fresh tubes. Centrifugation and transfer were repeated twice. Next, samples were digested with proteinase K as specified in QIAamp Fast DNA Stool Mini Kit handbook. Further steps were performed according to the handbook. DNA was analyzed by Qubit (Invitrogen, Thermo Fisher Scientific, Waltham, USA) and integrity was checked by agarose gel electrophoresis.

Illumina TruSeq DNA libraries were prepared using the TruSeq nano DNA Library preparation kit (Illumina, San Diego, USA) according to the manufacturer's instructions. Subsequently, libraries were checked for quality and library size on a 2100 Bioanalyzer instrument using a High Sensitivity DNA Assay kit (Agilent Technologies, Santa Clara, USA). The final libraries were quantified using a Quant-iT™ PicoGreen™ ds DNA Assay Kit (Thermo Fisher Scientific, Waltham, USA) and equimolarly pooled prior to sequencing. Subsequently, libraries were sequenced with Illumina NextSeq 500/550 platform and a high output v2 kit (150 cycles). The produced single-end reads which passed Illumina's chastity filter were subject to de-multiplexing and trimming of Illumina adaptor residuals using Illumina's bcl2fastq software version 2.20.0.422 (no further refinement or selection). Quality of the reads in fastq format was checked with the software FastQC (version 0.11.8). Sequenced reads were mapped to the NCBI nr protein database via the software Diamond (version 0.9.24) using standard parameters. The produced. daa alignment files were then “meganized”—linked to pathway databases—using the facilities provided by the software Megan (version 6). To estimate alpha diversity and richness, Shannon, Chao and Simpson diversity indexes were calculated and plotted using the phyloseq package.

The statistical difference between the faecal microbiota at the species level in HSPC and CRPC was calculated using the Limma package. In particular, the input fitted model was obtained by fitZig function of MetagenomeSeq R package. In order to compare the different cohorts, the differential abundance analysis was performed for UK (United Kingdom) and CH (Switzerland) cohorts separately. Only bacteria enriched in both UK and CH cohorts were considered. A meta-analysis for calculation of significance was performed using maximum function (metap R package) and adjusted p-value was used as the input for this meta-analysis. The threshold of significance for the meta p-value was 0.01. To highlight differential pathways in each group, the linear discriminant analysis (LDA) effect size (LEfSe) method was used with default settings on the website (https://huttenhower.sph.harvard.edu/galaxy/root) in CH cohort. Normalized read counts for differentially represented species in HSPC and CRPC gut microbiome were log-transformed and plotted as a heat map using the pheatmap package and the waterfall plots were plotted using ggplot2 R package. Analysis of survival was performed using survival R package and, specifically, using Kaplan-estimator and Cox-regression model. Log-rank test was used to calculate statistical significance in survival curves. For overall survival analysis (FIG. 15, A-I), the classification for presence or absence of bacteria was performed using decostand R function (method=“pa”) of vegan R package. The group “presence” corresponds to patients with simultaneous presence of bacteria influencing overall survival in CRPC cohort (RRSC). The group “absence” corresponds to patients with simultaneous absence of bacteria influencing overall survival in HSPC cohort (PPP).

Statistical analysis. Data were checked for normal distribution before any statistical analyses, and outliers excluded with Grubb's test. Data were analyzed with GraphPad Prism version 8.3.0. Values are presented as mean±s.e.m. and represented as individual values in scattered dot plots. Statistical significance between two groups was determined using two-tailed unpaired Student's t-test, while comparison among three or more groups was evaluated with one- or two-way ANOVA followed by Tukey's or Sidak's post-hoc test. A p value of P<0.05 was considered significant (*). For animal experiments, sample size was defined on the basis of past experience with the models. The minimum number of animals necessary to achieve the scientific objectives was used for ethical reasons. Animals were allocated randomly to treatment groups. Different treatment groups were processed identically and animals in different treatment groups were exposed to the same environment. In the immunohistochemistry and histopathological evaluations, investigators were blind during analysis.

Sequence listing part of the description:
NKX3.1 Forward
SEQ ID N 1
TCCGTCTTTTGGCTCTGAGT
NKX3.1 Reverse
SEQ ID N 2
GTGAAAGTGCACGCTGAAAA
R18S Forward
SEQ ID N 3
ACCGCAGCTAGGAATAATG
R18S Reverse
SEQ ID N 4
GCCTCAGTTCCGAAAACCA
ALDH1A3 Forward
SEQ ID N 5
GGGTCACACTGGAGCTAGGA
ALDH1A3 Reverse
SEQ ID N 6
CTGGCCTCTTCTTGGCGAA
PPAPA2A Forward
SEQ ID N 7
AGAGGGGCTTTTTCTGTACTGA
PPAPA2A Reverse
SEQ ID N 8
TATACGGGACGGGATGGTACT
R. gnavus Forward
SEQ ID N 9
TGGGTGTAAAGGGAGCGTAG
R. gnavus Reverse
SEQ ID N 10
CTCTCCGACACTCTAGCCTG
B. acidifaciens Forward
SEQ ID N 11
TTCGGTATGGGATGGGGATG
B. acidifaciens Reverse
SEQ ID N 12
CATCCTTCACGCTACTTGGC
EUB16S Forward
SEQ ID N 13
GGTGAATACGTTCCCGG
EUB16S Reverse
SEQ ID N 14
TACGGCTACCTTGTTACGA
8F primer
SEQ ID N 15
AGAGTTTGATCCTGGCTCAG
534R primer
SEQ ID N 16
ATTACCGCGGCTGCTGG

1.-16. (canceled)

17. A method for preventing and/or treating a disease caused by or characterized by an androgen deficiency comprising administering to a subject in need thereof a probiotic mixture comprising at least one bacterial strain selected from the genus Ruminococcus, the genus Clostridiales, the genus Sellimonas and the genus Drancourtella to the subject.

18. The method according to claim 17, further comprising at least one bacterial strain selected from the group consisting of the genus Streptococcus.

19. The method according to claim 17, comprising one or more or all of the following bacterial strains: Ruminococcus gnavus, Ruminococcus sp. DSM_100440, Ruminococcus sp. OM05_10BH, Streptococcus vestibularis, Clostridiales bacterium VE202_14, Sellimonas intestinalis and Drancourtella massiliensis.

20. A method for preventing, treating and/or inhibiting the development of prostate cancer in a subject in need thereof comprising administering a therapeutically effective amount probiotic mixture comprising at least one bacterial strain selected from the genus Prevotella to the subject.

21. The method according to claim 20, wherein the probiotic mixture comprises at least one bacterial strain selected from the group consisting of the genus Lactobacillus and/or the genus Bifidobacterium.

22. The method according to claim 20, wherein said prostate cancer is castration-resistant prostate cancer (CRPC).

23. The method according to claim 20 wherein the probiotic mixture comprises one or more of the following bacterial strains: Prevotella sp. BCRC_81118, Prevotella sp. Marseille_P4119, Prevotella sp. 885, Prevotella stercorea, L. casei, L. buchneri, L. acidophilus, L. paracasei, L. bulgaricus, L. rhammosum, B. bifidum, B. longum, and/or B. breve.

24. The method according to claim 20, further comprising administering one or more of the following antibiotics: vancomycin, ampicillin, neomycin, and/or metronidazole.

25. A method for preventing, treating and/or inhibiting the development of prostate cancer comprising administering to a subject in need thereof a postbiotic mixture comprising at least one inactivated bacterial strain selected from the genus Prevotella to the subject.

26. The method according to claim 25, wherein said prostate cancer is castration-resistant prostate cancer (CRPC).

27. The method according to claim 25, wherein said postbiotic mixture comprises at least one inactivated bacterial strain is Prevotella stercorea.

28. A method for preventing, treating and/or inhibiting the development of prostate cancer in a subject in need thereof comprising administering a therapeutically effective amount of the probiotic mixture according to claim 20 and a drug for androgen deprivation therapy to the subject.

29. A method for preventing, treating and/or inhibiting the development of prostate cancer in a subject in need thereof comprising administering a therapeutically effective amount of the postbiotic mixture according to claim 25 and a drug for the androgen deprivation therapy to the subject.

30. The method according to claim 28, wherein said drug for the androgen deprivation therapy is selected from a LHRH analog or antagonist.

31. A method for preventing, treating and/or inhibiting the development of prostate cancer comprising administering to a subject in need thereof a therapeutically effective amount of the probiotic mixture according to claim 20 and a drug for the hormonal therapy to the subject.

32. A method for preventing, treating and/or inhibiting the development of prostate cancer comprising administering to a subject in need thereof a therapeutically effective amount of the postbiotic mixture according to claim 25 and a drug for the hormonal therapy to the subject.

33. The method of claim 31, wherein said drug for the hormonal therapy is selected from Bicalutamide, Enzalutamide, Abiraterone, or Apalutamide.

34. (canceled)

35. A method of inhibiting the development of prostate cancer in a subject comprising administering a therapeutically effective amount of a pharmaceutical composition comprising vancomycin, ampicillin, neomycin, and/or metronidazole to the subject.

36. The method of claim 20, further comprising administration of the probiotic mixture of claim 20 and at least one of vancomycin, ampicillin, neomycin, and/or metronidazole.

37. The method according to claim 34, wherein said prostate cancer is a castration-resistant prostate cancer.

38. The method according to claim 29, wherein said drug for the androgen deprivation therapy is selected from a LHRH analog or antagonist.

39. The method of claim 32, wherein said drug for the hormonal therapy is bicalutamide, enzalutamide, abiraterone, or apalutamide.
